(19) United States
(12) Patent Application Publication
(10) Pub. No.: US 2025/0354175 A1
(43) Pub. Date: Nov. 20, 2025

(54) ATTP MV4-DERIVED SITE-SPECIFIC RECOMBINATION AND ITS USE FOR INTEGRATION OF SEQUENCE OF INTEREST

(71) Applicants: INSTITUT NATIONAL DE RECHERCHE POUR L'AGRICULTURE, L'ALIMENTATION ET L'ENVIRONNEMENT, PARIS (FR); CENTRE NATIONAL DE LA RECHERCHE SCIENTIFIQUE, PARIS (FR); INSTITUT NATIONAL DES SCIENCES APPLIQUEES DE TOULOUSE, Toulouse Cedex 4 (FR); UNIVERSITE PAUL SABATIER TOULOUSE III, Toulouse (FR)

(72) Inventors: Pascal LE BOURGEOIS, GIBEL (FR); Kevin DEBATISSE, Toulouse (FR); Muriel COCAIGN-BOUSQUET, Mons (FR)

(21) Appl. No.: 18/872,961

(22) PCT Filed: Jun. 2, 2023
(86) PCT No.: PCT/EP2023/064892

§ 371 Date: Dec. 9, 2024

(30) Foreign Application Priority Data
Jun. 7, 2022 (EP) 22305825.6

(51) Int. Cl. C12N 15/90; C12N 15/70

(52) U.S. Cl. CPC C12N 15/902; C12N 15/70

(57) ABSTRACT

The present disclosure relates to a method for preparing a site-specific recombination polynucleotide molecule derived from the attP site of the bacteriophage mv4 and to a kit for such site-specific recombination. The kit can be used to transform procaryote hosts to integrate any polynucleotide sequence of interest.

### CROSS-REFERENCE TO RELATED APPLICATION(S)

This present application is a national stage application of International Patent Application No. PCT/EP2023/064892, filed Jun. 2, 2023, which claims priority to European Patent Application No. 22305825.6, filed Jun. 7, 2022, the disclosures of which are hereby incorporated by reference in their entireties.

### TECHNICAL FIELD

The present invention relates to a method for preparing a site-specific recombination polynucleotide molecule derived from the attP site of the bacteriophage mv4 and to a kit for such site-specific recombination. The kit can be used to transform prokaryote hosts to integrate any polynucleotide sequence of interest.

### BACKGROUND

Viruses are the most abundant biological entities on Earth, with about 4.10³⁰ viruses in the ocean water. Among viruses, bacteriophages are the most abundant organisms with an estimation of more than 10³⁰ tailed phages in the biosphere, outnumbering bacteria by a factor of about 10 to 1. Temperate bacteriophages are obligate parasites of bacterial cells that can mediate two distinct lifecycles, the lytic and lysogenic cycles. During the infection, these phages usually proceed to the establishment of a lytic cycle, where the viruses hijack the host-cell machinery to replicate their DNA, to assemble viral particles and to allow their dissemination in the environment by lysis of their host. However, depending on several environmental factors, temperate phages are able to proceed through a lysogenic cycle resulting in repression of phage's genes expression and integration of the viral genome at a single site of its host chromosome. This site-specific recombination involves site-specific recombinases (SSR) that promote DNA rearrangements between two specific DNA target sites (Grindley et al., 2006). Once integrated, the bacteriophage genome, called prophage, is passively replicated along with the bacterial chromosome. On some occasions, such as exposition to some chemicals or accumulation of DNA-damages, prophage DNA excises from the bacterial chromosome and reactivates its lytic cycle. Phage DNA integration and excision are mediated by a phage-encoded protein called integrase, a SSR mostly belonging to the heterobivalent tyrosine recombinases (YR) subfamily although an increasing number of integrases are members of the serine integrases, a phylogenetically and mechanistically unrelated SSR family (Grindley et al., 2006).

All integrases catalyse the unidirectional recombination between two dedicated sites, one present on the phage DNA (attP) and one located on the bacterial host cell chromosome (attB), leading to the integrated prophage flanked by hybrids recombination sites (attL and attR). The site-specific recombination is associated to the presence of almost identical 20 to 40-pb DNA segments in attP and attB sites, called the “core” region. This region is made of two imperfect inverted repeats, where integrases monomers bind, that flanks an “overlap” region where DNA breakage and religation occur. In most cases, sequence identity within the “core” region is critical for the recombination, with consequently a very low “off-target” activity compared to nuclease-based systems such as CRISPR/Cas9, ZFNs or TALEN.

By providing a mean to insert or delete DNA regions with high fidelity and high efficiency, site-specific recombinases (SSR) have been widely used for genetic manipulation of living organisms, from bacteria to mammalian cells. Among the different systems actually used for genome engineering, the best known are the tyrosine-recombinases (YR) Cre/loxP from the E. coli bacteriophage P1 and integrase from the lambda phage (^{λ}Int), respectively used for gene knock-in in eukaryotic cells and the Gateway™ cloning system. However, integrases have two main drawbacks that severely limit applications of such systems in genome engineering: i) lambda-like integrases depend on specific host-factor to recombine, excluding their use outside the bacterial species they originate, and ii) site-specific recombinases require specific DNA sites that cannot be easily modified because DNA sites and integrases co-evolved together.

A more flexible system using SSR is needed to be able to integrate a foreign DNA into a prokaryotic genome. Indeed, insertion of foreign DNA into a genome is actually only achievable if cognate attB site or pseudo-attB sites are present into this genome, or if cognate attB site has been previously introduced into the recipient genome by other methods (e.g. homologous recombination) to generate “landing pads”.

From the prior art, it is known to redirect recombination by using gRNA specific for a target site, WO2020181264, for example, describes an integration system using an RNA guide and the CRISPR-Cas system or by using modified integrases; in the WO2020165901, the Inventors have developed variants and mutants of the HK022 bacteriophage integrase (YR) in three specific domains for enhanced target replacements in eukaryotic cells.

The temperate bacteriophage mv4 (Mata et al., 1986; Cluzel et al., 1987) integrates its DNA at the 3′-end of the tRNA^{SER}(CGA) locus of the Lactobacillus delbrueckii subsp. bulgaricus chromosome by site-specific recombination (Dupont et al., 1995a). The site-specific recombination module of the bacteriophage mv4 is made of an integrase (^{mv4}Int) that belongs to the heterobivalent YR subfamily and that catalyses the recombination between a 234-bp attP phage DNA site (the donor site) and an atypical bacterial 16-pb DNA site attB (the target site) (Coddeville et al., 2014a). In contrast to many classical YR, the “^{mv4}Int/attP” system is able to drive recombination in a wide range of bacteria, including E. coli and various Gram-positive species (Auvray et al., 1997), indicating that it does not depend of species-specific host-factor to promote recombination and thus might be used as a generic system for in vivo DNA recombination. In addition, both attP and attB sites have atypical organization, with unusual location of arm-binding sites on attP, absence of ^{mv4}Int core-binding sites into the 17-bp core sequences common to attP and attB, and a noncanonical 8-bp overlap sequence (Coddeville et al., 2014a).

Interestingly, the Inventors demonstrate that the ^{mv4}Int can be reprogramed to integrate DNA plasmid by site-specific recombination into bacterial host attB site by adapting the core-attP region (redefined as a 21-bp sequences) of the attP donor site to the newly defined 21-bp attB target site. This result has been established by finely defining and modifying the nucleotides of the so-called “overlap” region, the region where the strand-exchange occurs, of the donor site attP; this system advantageously avoids any genetic manipulation of the bacterial host genome.

The Inventors also demonstrated that by adapting attP site to any bacterial attB site, they are able to integrate DNA into the bacterial chromosome of different bacteria and without needing any host factors.

### SUMMARY

The present invention relates to a method for preparing a site-specific recombination polynucleotide molecule comprising the steps of:
a—selecting a DNA target site in the genome of a bacterial host cell having a sequence of B—O—B′ wherein:
B is 5′-X1-X1-X2-X3-X3-X3-X4-3′ wherein at most 1 of the nucleic acids of B may be N;
O is 5′-N—N—N—N—N—N—N-3′ and
B′ is 5′-X1-X5-X5-X5-X6-X7-X2-3′ wherein at most 1 of the nucleic acids of B′ may be N; wherein X1 to X7 and N have independently the following definitions:
X1 is A or G or T;
X2 is C or G or T;
X3 is A or G;
X4 is A or T;
X5 is C or T;
X6 is A or C or G;
X7 is A or C or T; and
N is A or C or G or T;
b—providing the site-specific recombination polynucleotide molecule having a sequence of C—O—C′ wherein
C is 5′-X1-X1-X2-X1-X3-X1-X4-3′ wherein at most 1 of the nucleic acids of C may be N;
O is 5′-N—N—N—N—N—N—N-3′; and
C′ is 5′-X1-X5-X5-X5-X6-X7-X5-3′ wherein at most 1 of the nucleic acids of C′ may be N;
and wherein X1, X2, X3, X4, X5, X6, X7 and N are as defined previously;
and wherein O of C—O—C′ is identical to O of B—O—B′ of the bacterial host cell.

In one embodiment, the method for preparing a site-specific recombination polynucleotide molecule comprises the steps of:
a—selecting a DNA target site in the genome of a bacterial host cell having a sequence of B—O—B′ wherein:
B is 5′-X1-X1-X2-X3-X3-X3-X4-3′ wherein at most 1 of the nucleic acids of B may be N;
O is 5′-N—N—N—N—N—N—N-3′ and
B′ is 5′-X4-X5-X5-X5-X6-X7-X2-3′ wherein at most 1 of the nucleic acids of B′ may be N;
b—providing the site-specific recombination polynucleotide molecule having a sequence of C—O—C′ wherein
C is 5′-X1-X1-X8-X1-X3-X1-X4-3′ wherein at most 1 of the nucleic acids of C may be N;
O is 5′-N—N—N—N—N—N—N-3′; and
C′ is 5′-X4-X5-X5-X5-X9-X7-X5-3′ wherein at most 1 of the nucleic acids of C′ may be N; and wherein X1 to X7 and N have independently the following definitions:
X1 is A or G or T;
X2 is C or G or T;
X3 is A or G;
X4 is A or T;
X5 is C or T;
X6 is A or C or G;
X7 is A or C or T;
X8 is G or T;
X9 is A or C; and
N is A or C or G or T;
and wherein O of C—O—C′ is identical to O of B—O—B′ of the bacterial host cell.

The recitation “wherein at most 1 of the nucleic acids of B may be N” means that, by exception to the definition of the sequence of B, one given nucleic acid Xn of B may have a different definition and be defined by N; the same applies to B′, C and C′.

By “independently”, it is meant that the value of a given nucleic acid Xn may be different within a sequence and from the other sequences. For example, the nucleic acid X1 of the first position of B may be different from the nucleic acid X1 of the second position of B and from the nucleic acid X1 of the first position of C.

Advantageously, because the method of the invention does not need any bacterial host factors, it can be used in any kind of bacterial host whether it is a Gram-positive or a Gram-negative bacterium.

For example, such method has successfully been used in Escherichia coli, Lactococcus lactis and Lactobacillus delbrueckii ssp. bulgaricus.

Preferably B is chosen among the 117 sequences indicated in the table 1 below:

| | Sequence |
|---|---|
| 1 | ATGGAAA |
| 2 | GTGGAAA |
| 3 | AAGGAAA |
| 4 | GAGGAAA |
| 5 | AAGGAAT |
| 6 | AAGGAGT |
| 7 | GTGGAAT |
| 8 | AGGGAAA |
| 9 | GGGGAAA |
| 10 | GTGGAGT |
| 11 | AAGAGAA |
| 12 | GTGAGAA |
| 13 | AAGGAGA |
| 14 | GAGGAAT |
| 15 | AATAGAA |
| 16 | ATGGAAT |
| 17 | GTCAGAA |
| 18 | ATGGAGT |
| 19 | AAGAAAA |
| 20 | GTGGAGA |
| 21 | TAGGAAA |
| 22 | CAGGAAA |
| 23 | ATGAGAA |
| 24 | GGGAAAA |
| 25 | TTCAGAA |
| 26 | GTCAAAA |
| 27 | GAGGAGT |
| 28 | GACAGAA |
| 29 | TATAGAA |
| 30 | GTGAAAA |
| 31 | AAGAAAT |
| 32 | GAGAAAA |
| 33 | GATAGAA |
| 34 | GGGGAAT |
| 35 | ATTAGAA |
| 36 | ATGGAGA |
| 37 | TGCAGAA |
| 38 | TGGGAAA |
| 39 | TTGGAAA |
| 40 | TTCAAAT |
| 41 | GAGAGAA |
| 42 | GGGGAGT |
| 43 | GTTAGAA |
| 44 | AAAGAAA |
| 45 | GGTAGAA |
| 46 | GGCAGAA |
| 47 | TACAGAA |
| 48 | AGGGAAT |
| 49 | GAGGAGA |
| 50 | AACAGAA |
| 51 | ATGAAAA |
| 52 | GTCAGAT |
| 53 | TTCAAAA |
| 54 | AAGAGAT |
| 55 | GTGAGAT |
| 56 | CAGAGAA |
| 57 | TACAAAA |
| 58 | GGGAGAA |
| 59 | ATCAGAA |
| 60 | AGCAGAA |
| 61 | AGGAAAA |
| 62 | AAGAAGT |
| 63 | AGTAGAA |
| 64 | GTCAAAT |
| 65 | AAGGAAC |
| 66 | TGTAGAA |
| 67 | CGGGAAA |
| 68 | CAGGAAT |
| 69 | TAGGAAT |
| 70 | TGCAAAA |
| 71 | AGGGAGT |
| 72 | GTGGAAC |
| 73 | CGCAGAA |
| 74 | AGGAGAA |
| 75 | CAGGAGT |
| 76 | TTTAGAA |
| 77 | AAAAGAA |
| 78 | GTGTAAA |
| 79 | CTGGAAA |
| 80 | TAGAGAA |
| 81 | GGCAAAA |
| 82 | AATAAAA |
| 83 | GAGAAAT |
| 84 | AATGAAA |
| 85 | GGTAAAA |
| 86 | AGGGAGA |
| 87 | GTCAGGT |
| 88 | AAGTAAA |
| 89 | GTCAACT |
| 90 | ACGGAAA |
| 91 | ATGGAAC |
| 92 | TGCAGAT |
| 93 | GTGAAAT |
| 94 | TAGGAGT |
| 95 | GATAAAA |
| 96 | TTCAGAT |
| 97 | AAGAAGA |
| 98 | GGGAAAT |
| 99 | AATAGAT |
| 100 | AAGGATA |
| 101 | GACAAAA |
| 102 | AGCAAAA |
| 103 | AACAAAA |
| 104 | ATCAAAA |
| 105 | CAGAAAA |
| 106 | CGTAGAA |
| 107 | ATGAGAT |
| 108 | GGGGAGA |
| 109 | CACAGAA |
| 110 | CTCAGAA |
| 111 | GTTAGAT |
| 112 | GGCAGAT |
| 113 | AAGAGGT |
| 114 | GTCGAAA |
| 115 | GTGGATA |
| 116 | ATGTAAA |
| 117 | CATAGAA |

Preferably B′ is chosen among the 140 sequences indicated into the table 2 below:

| | Sequence |
|---|---|
| 1 | ATTCCTA |
| 2 | ACTTACC |
| 3 | ACCTCTT |
| 4 | TTCTCTT |
| 5 | ACTCCTA |
| 6 | TTTTGTC |
| 7 | ATCTGCC |
| 8 | TTCTGTC |
| 9 | GTTCCAC |
| 10 | ATCTGTC |
| 11 | GCTCCAT |
| 12 | ATCTGAC |
| 13 | TTTCCTT |
| 14 | ACTTCCT |
| 15 | ATCTGAT |
| 16 | ATCTCTA |
| 17 | TTCTCTC |
| 18 | TTCTCCC |
| 19 | TTTCACT |
| 20 | TTCTGAC |
| 21 | ATCTATT |
| 22 | ACTTAAT |
| 23 | ATTCATT |
| 24 | TTTACAC |
| 25 | ATCTGTT |
| 26 | TTTCCTG |
| 27 | TTTTCTC |
| 28 | TCTCCTG |
| 29 | ATCTATC |
| 30 | ATTTCAC |
| 31 | ATTTATC |
| 32 | TATTCTT |
| 33 | ATCTGCA |
| 34 | TTCTGCG |
| 35 | TTTCCCA |
| 36 | AGTTGCG |
| 37 | TTTCTAT |
| 38 | ATTCCTT |
| 39 | ACTCCGT |
| 40 | AGTTGAC |
| 41 | TCTTCTG |
| 42 | TTCTACG |
| 43 | AGTTCAT |
| 44 | AGTTCTT |
| 45 | ACTCTTT |
| 46 | ACTTCTT |
| 47 | ACTCCTG |
| 48 | ATTTGCG |
| 49 | TTTTCTG |
| 50 | TTCTTTC |
| 51 | ATCTAAC |
| 52 | ATTCCTC |
| 53 | ATTTAAT |
| 54 | ATCTACT |
| 55 | TTTTATC |
| 56 | ATCTCCC |
| 57 | ATTTGTT |
| 58 | AATCCAC |
| 59 | ATTTATT |
| 60 | ACTCCTT |
| 61 | TTTCCAG |
| 62 | TTTACCT |
| 63 | GTTTCTC |
| 64 | GTTCCCT |
| 65 | GTTCCAT |
| 66 | ATTTGTC |
| 67 | TTTACTC |
| 68 | AGTTATT |
| 69 | ATTACTT |
| 70 | TTTCCCG |
| 71 | TTTACAT |
| 72 | ATCTACC |
| 73 | TTTCATT |
| 74 | TATCCTT |
| 75 | ATTTAAC |
| 76 | ACCTCAC |
| 77 | ATTCCCG |
| 78 | AGTTGAT |
| 79 | TTCTGCT |
| 80 | AATTCTT |
| 81 | TTTTGAT |
| 82 | TTCTCAT |
| 83 | TTCTAAT |
| 84 | ATCTTTT |
| 85 | TCTTCTC |
| 86 | TCTCCCT |
| 87 | TTTCCAT |
| 88 | AATCCTC |
| 89 | ACTTGTT |
| 90 | GTCTATC |
| 91 | ATCTCAT |
| 92 | TTTTCCC |
| 93 | TATTCCC |
| 94 | AGTTACC |
| 95 | AATCCTT |
| 96 | GTTCCTC |
| 97 | ATTACTC |
| 98 | AGTTGTT |
| 99 | ATCTGTG |
| 100 | ATGTCAC |
| 101 | TCTCCTT |
| 102 | ACTCCCT |
| 103 | ACTTCAC |
| 104 | ATTCCTG |
| 105 | AATTCCC |
| 106 | TCTTCCT |
| 107 | TTTCCTC |
| 108 | ATTCACC |
| 109 | ATTACAA |
| 110 | ATTCCAC |
| 111 | TCTCCGT |
| 112 | TTCTCTA |
| 113 | ACTTCCC |
| 114 | TTTCACC |
| 115 | AATCCAT |
| 116 | TTTCTAC |
| 117 | TCTCCAG |
| 118 | ATCTCAC |
| 119 | TTTTCAC |
| 120 | ATTACAC |
| 121 | TCCTATT |
| 122 | ATTTAAG |
| 123 | TTTCCCT |
| 124 | ATTTCCC |
| 125 | ATTCGCC |
| 126 | GTTACTT |
| 127 | GTTCCTG |
| 128 | TTCTTCC |
| 129 | TCCTCAC |
| 130 | TATCCAT |
| 131 | TCCTCTT |
| 132 | ATTCCCC |
| 133 | ATTTCCT |
| 134 | ACCTCCT |
| 135 | ACTACTT |
| 136 | GTTCCTT |
| 137 | ATTCCAG |
| 138 | TATCCTC |
| 139 | ATTTCTT |
| 140 | TTCTCAC |

Preferably C is chosen among the 221 sequences indicated into the table 3 below:

| | Sequence |
|---|---|
| 1 | GGTAAAA |
| 2 | GTGGAAA |
| 3 | TTGGAAA |
| 4 | TTGGAGA |
| 5 | GTGGAAT |
| 6 | GGGTAAA |
| 7 | GTGAGGA |
| 8 | AGGGAGT |
| 9 | GGTTAAA |
| 10 | ATGGATA |
| 11 | GTTAGAT |
| 12 | GGGGAGA |
| 13 | GGGGGAA |
| 14 | GGGTAGA |
| 15 | TGTGAAA |
| 16 | TGTAGGA |
| 17 | GTGGAGA |
| 18 | AGTAATA |
| 19 | AGGGGAA |
| 20 | ATGTAAA |
| 21 | GTGAGGT |
| 22 | GTGTAAA |
| 23 | GGGAATA |
| 24 | GGTAAGA |
| 25 | GGGAGGA |
| 26 | GTTAGGT |
| 27 | GGGAGGT |
| 28 | TATAGGT |
| 29 | TGGGAAT |
| 30 | TGGGAGT |
| 31 | GTGAGAT |
| 32 | GGTAATA |
| 33 | GGTGGAA |
| 34 | GAGTAAA |
| 35 | TATAGGA |
| 36 | ATGAGGT |
| 37 | GTGGAGT |
| 38 | AATGGGT |
| 39 | GTGGATT |
| 40 | TGTAGGT |
| 41 | TATAGAT |
| 42 | GTGAATA |
| 43 | ATTGGAA |
| 44 | GTGAGTT |
| 45 | GGTAGGT |
| 46 | TTTAAAA |
| 47 | GGGGAGT |
| 48 | AAGGGGT |
| 49 | AATGAGT |
| 50 | TGGTAAA |
| 51 | GTGTAGA |
| 52 | ATTAGAT |
| 53 | GTGTATA |
| 54 | ATGGATT |
| 55 | GTTAATT |
| 56 | ATGAGGA |
| 57 | GGGGAAT |
| 58 | TTTAGAT |
| 59 | GTTAGTA |
| 60 | GTTAGGA |
| 61 | GGTAAAT |
| 62 | GTTGGAA |
| 63 | ATTTAAA |
| 64 | AGGGATT |
| 65 | ATTAGGT |
| 66 | TAGAGGA |
| 67 | GATGGGT |
| 68 | TAGAGTA |
| 69 | GGTAGGA |
| 70 | GGTGAAT |
| 71 | GGGGATT |
| 72 | GGTAAGT |
| 73 | GGGAAGT |
| 74 | TTGTAAA |
| 75 | AAGGATT |
| 76 | AGTAATT |
| 77 | TTGGAAT |
| 78 | ATGGGGA |
| 79 | GAGGATT |
| 80 | ATTAATT |
| 81 | GGGTAAT |
| 82 | GTGAAGT |
| 83 | GTTTGAA |
| 84 | AGGAGGT |
| 85 | GTGGGAT |
| 86 | GTGAATT |
| 87 | TGTAATA |
| 88 | GTTGATA |
| 89 | ATGGGAT |
| 90 | GTGTAAT |
| 91 | GGTGAGA |
| 92 | GTGAGTA |
| 93 | TTTGAAA |
| 94 | GTTAAAA |
| 95 | AATAATA |
| 96 | GTGGTAA |
| 97 | GATGAGT |
| 98 | TTGGATA |
| 99 | ATTAAGT |
| 100 | ATTGAGT |
| 101 | GAAAGAA |
| 102 | ACTGAAA |
| 103 | CATAGAA |
| 104 | ATCACAA |
| 105 | AGGAAAA |
| 106 | TACAGAT |
| 107 | GAGAGAA |
| 108 | GATAAAA |
| 109 | CCTAGAA |
| 110 | ACGGAAA |
| 111 | CTGAGAA |
| 112 | AAGCAAA |
| 113 | CAGGAAA |
| 114 | ACGTAAA |
| 115 | CGCAGAA |
| 116 | GGCAGAA |
| 117 | TGCACAA |
| 118 | TGCAGAA |
| 119 | CGGGAAA |
| 120 | CAGAAAA |
| 121 | TGCAGAT |
| 122 | GAGAGGA |
| 123 | ATCAATC |
| 124 | ACAAGAA |
| 125 | GACGAAA |
| 126 | GATAGAT |
| 127 | CTGGAAA |
| 128 | ATGAGTA |
| 129 | CGGAGAT |
| 130 | ATCTGAA |
| 131 | GGGAGAA |
| 132 | AAAAGAA |
| 133 | ACCGAAA |
| 134 | GTGGATA |
| 135 | GATAGAA |
| 136 | ATCAAAA |
| 137 | CATAGAT |
| 138 | CAGTAAA |
| 139 | AATAGAT |
| 140 | AGTAGGA |
| 141 | ATCAGAA |
| 142 | CATAAAA |
| 143 | AGGGAAA |
| 144 | ATCAGGA |
| 145 | AGCAGAA |
| 146 | CCGGAAA |
| 147 | TAAAGAA |
| 148 | CAGAGAT |
| 149 | GATAATA |
| 150 | ACGGAGA |
| 151 | AGCGAAA |
| 152 | GTGAAGA |
| 153 | AAGAAGA |
| 154 | AGGGAGA |
| 155 | CACAATA |
| 156 | ATCAGGT |
| 157 | GAAGAAT |
| 158 | TACGAAA |
| 159 | GACAGGT |
| 160 | AGGTAAA |
| 161 | GTAAGAA |
| 162 | GTCAGAA |
| 163 | AAAGATA |
| 164 | GATAAGA |
| 165 | AACAGTA |
| 166 | GAGGATA |
| 167 | GACAGAA |
| 168 | AAGTAAA |
| 169 | AGGAAGA |
| 170 | TGCAAAT |
| 171 | ATAGAGA |
| 172 | AAGGAGA |
| 173 | TGAAGAA |
| 174 | AGCTAAA |
| 175 | ATGAGAT |
| 176 | AGTAGTA |
| 177 | GAGGAAA |
| 178 | TAGAGAA |
| 179 | ACCAGAT |
| 180 | GACAGAT |
| 181 | AATAGGT |
| 182 | AGTAGAT |
| 183 | AACAGGA |
| 184 | TCGGAAA |
| 185 | GGCGAAA |
| 186 | AACAGAA |
| 187 | GTGACGA |
| 188 | TAGAGAT |
| 189 | GGCTAAA |
| 190 | TCTAGAA |
| 191 | ATCAATA |
| 192 | ACGAGAT |
| 193 | GAGAAGA |
| 194 | AGAAGAA |
| 195 | ATGAGAA |
| 196 | TATAAAA |
| 197 | ACCTAAA |
| 198 | GAGGGAA |
| 199 | TTCAGAT |
| 200 | CTCAGGT |
| 201 | TACAGAA |
| 202 | GCGGAGA |
| 203 | AACAAAA |
| 204 | AAAGGAA |
| 205 | AGGAGAT |
| 206 | CGGGAGA |
| 207 | TAGGAAA |
| 208 | TCGAGAA |
| 209 | CTTAGAT |
| 210 | CACAGTA |
| 211 | TGGGAAA |
| 212 | AATACAA |
| 213 | ATGGAGA |
| 214 | TAGGAGT |
| 215 | CGCGAAA |
| 216 | GTCAGGA |
| 217 | AATAGTA |
| 218 | GTCAGGT |
| 219 | CGCAATT |
| 220 | GCGGAAA |
| 221 | GATGAAA |

Preferably C′ is chosen among the 161 sequences indicated into the table 4 below:

| | Sequence |
|---|---|
| 1 | ATTCCTT |
| 2 | TCTCCAT |
| 3 | TTTCCAC |
| 4 | ATTCCAC |
| 5 | ACTCCAT |
| 6 | TTTCCAT |
| 7 | TTTTCTT |
| 8 | ACTTCTT |
| 9 | TTTCCTT |
| 10 | ACTCCTA |
| 11 | ATTCCAT |
| 12 | ATTTCCC |
| 13 | ATTTCTT |
| 14 | TTTTCCC |
| 15 | ATTCCTC |
| 16 | TCTCCTT |
| 17 | ATCTCTT |
| 18 | GTTCCTT |
| 19 | TCTCCAC |
| 20 | ATTCCTG |
| 21 | TCTTCTT |
| 22 | ACTTCTC |
| 23 | ATCTCAT |
| 24 | GTTCCAT |
| 25 | ATTTCAC |
| 26 | TTTTCAC |
| 27 | TTTTCTC |
| 28 | TTCTCAC |
| 29 | TCTTCTC |
| 30 | ATTTCAT |
| 31 | GCTCCTT |
| 32 | GTTTCTT |
| 33 | GTTCCAC |
| 34 | TTCTCAT |
| 35 | TTTTCCT |
| 36 | TTCTCTT |
| 37 | TTTACTT |
| 38 | TTTCCTG |
| 39 | ATCTATT |
| 40 | ACTTCCC |
| 41 | ATTACTT |
| 42 | TATCCTT |
| 43 | ATCTAAT |
| 44 | TTTTCTG |
| 45 | TTTTCAT |
| 46 | ACTTCAT |
| 47 | TTTCTTT |
| 48 | TATTCTT |
| 49 | ATCTATC |
| 50 | TTTCCTA |
| 51 | ATTTATT |
| 52 | ACTTATT |
| 53 | ACCTCAT |
| 54 | ATTTACC |
| 55 | ATTCTTT |
| 56 | ACCTCTT |
| 57 | TTTTATT |
| 58 | TTTCCGT |
| 59 | ATCTGTC |
| 60 | ACTACTT |
| 61 | ACTCCGT |
| 62 | ATCTGTT |
| 63 | AATCCAT |
| 64 | ATCTGCT |
| 65 | TTCTATT |
| 66 | ATCTACT |
| 67 | AATCCTT |
| 68 | ATTACAT |
| 69 | AGTTATT |
| 70 | ATCTCTG |
| 71 | TCTCCTA |
| 72 | TTTACAT |
| 73 | ATTTCTG |
| 74 | ATTTGCT |
| 75 | AGTTCTT |
| 76 | GTCTCTT |
| 77 | TTTACAC |
| 78 | ATTCCGT |
| 79 | ATTTATC |
| 80 | TCTTCCT |
| 81 | ACTCCCG |
| 82 | TTCTCTG |
| 83 | GTCTCAC |
| 84 | TCTTCAC |
| 85 | TATCCAT |
| 86 | ATTTCTA |
| 87 | GTTTCCC |
| 88 | TTCTATC |
| 89 | TTTTATC |
| 90 | TTCTAAT |
| 91 | TTCTGTT |
| 92 | ATTCCGC |
| 93 | ATTTGTT |
| 94 | TTCTGAT |
| 95 | AGTTAAT |
| 96 | ACTACAC |
| 97 | ACCTATT |
| 98 | AGTTCAC |
| 99 | ACTTATC |
| 100 | TTTACCC |
| 101 | ACTCCAC |
| 102 | ATTTCTC |
| 103 | ATTCCCT |
| 104 | ACTCCTT |
| 105 | TTTCCCC |
| 106 | ACTCCTC |
| 107 | ATTCCCC |
| 108 | ATCTGAT |
| 109 | ACTCCTG |
| 110 | ACTTCCT |
| 111 | TTTCCTC |
| 112 | TTTCCCT |
| 113 | ATCTCTC |
| 114 | ATTCCTA |
| 115 | AATTCTT |
| 116 | ATCTCAC |
| 117 | ACTCCAG |
| 118 | GTTCCTC |
| 119 | TCTTCCC |
| 120 | TCTCCTC |
| 121 | ACTCCCA |
| 122 | ACTTCAC |
| 123 | ACTTACC |
| 124 | ATCTGAC |
| 125 | ACTCTTT |
| 126 | ATTTCCT |
| 127 | ATCTCTA |
| 128 | TCCTCAC |
| 129 | TTCTCTC |
| 130 | ATCTCCC |
| 131 | GCTCCAT |
| 132 | ACTCCGC |
| 133 | TTTCCAA |
| 134 | GTTCCCC |
| 135 | TTTCCAG |
| 136 | GCTTCTT |
| 137 | AATCCAC |
| 138 | ATCTACC |
| 139 | ATCTGCG |
| 140 | ATCTGCC |
| 141 | ATTACAC |
| 142 | ATTCCCG |
| 143 | ATCTCCT |
| 144 | TCTCCCC |
| 145 | ATCTAAC |
| 146 | TATCCAC |
| 147 | ACTCCAA |
| 148 | TTCTGCG |
| 149 | ACTACAT |
| 150 | GTCTCAT |
| 151 | TTTCCGC |
| 152 | ACTTCTG |
| 153 | ACTACCC |
| 154 | ATTCCAA |
| 155 | TCTCCTG |
| 156 | TTCTGCC |
| 157 | ATTCCCA |
| 158 | TTTCCCG |
| 159 | ATCTACG |
| 160 | GCTCCCC |
| 161 | TCTCCAG |

In one embodiment, the method for preparing a site-specific recombination polynucleotide molecule of the invention is such that: C is 5′-GAAAGAA-3 and C′ is 5′-TCTCCTT-3′;

In such embodiment, C and C′ correspond to their wild type sequences.

In another embodiment, the method for preparing a site-specific recombination polynucleotide molecule of the invention is such that: C is 5′-GAAAGAA-3 and B′ and C′ have the same sequence.

Preferably, in such embodiment, C corresponds to its wild type sequence and the DNA target is the native target of the ^{mv4}Int: tRNA^{SER}(CGA) of the Lactobacillus delbrueckii subsp. bulgaricus (SEQ ID No 1). More preferably in this embodiment, B corresponds to its wild type sequence.

The present invention further relates to a kit for site-specific recombination of at least one polynucleotide sequence of interest into the genome of a bacterial host cell comprising:
A—a polynucleotide molecule A comprising:
(i) a sequence of between 220 to 250 pb comprising the polynucleotide fragments P1-P2, C—O—C′ and P′1-P′2 wherein:

P1-P2 is
(SEQ ID No 2)
5′-ATCAACTAGATTTTTAACTAGAA-3′;

C—O—C′ is the site-specific recombination polynucleotide molecule as defined with the method of the invention; and

P'1-P'2 is
(SEQ ID No3 )
5′-TTTAACTAGAAAATAACTAGAA-3′;

said sequence interacting with the DNA target site in the genome of the bacterial host cell having a sequence of B—O—B′ for integrating the polynucleotide sequence of interest into the bacterial chromosome; and
(ii) at least one polynucleotide sequence of interest;
B—a polynucleotide molecule int having at least 80%, preferably at least 85%, 90%, 95% or 100% identity with the sequence of SEQ ID No 4 coding for ^{mv4}Int or the ^{mv4}Int of SEQ ID No 5.

The sequence O of C—O—C′ and the sequence O of B—O—B′ are identical, allowing the overlap of the polynucleotide molecule A and the DNA target site. This overlap induces an integration of the polynucleotide sequence of interest into the bacterial DNA.

The polynucleotide molecule A comprises polynucleotides fragments P1-P2, C—O—C′ and P′1-P′2, preferably organized as follows: P1-P2-Nₙ—C—O—C′—Nₙ′—P′1-P′2. P sites are the ^{mv4}Int arm-type binding sites, P1-P2 are the sites for the left arm and P′1-P′2 are the sites for the right arm. Those sites surround a core region of 21 pb defined by C, O and C′. O is the overlap region. n and n′ represent a whole number of nucleic acids N, N being A, T, G or C.

The polynucleotide molecule A has a size of between 220 to 250 pb, preferably 234 pb.

The term “polynucleotide sequence of interest” means any polynucleotide sequence.

The method allows the integration of sequences involved in various functions and pathways.

In a specific illustrative embodiment, the polynucleotide sequence of interest can be defined as a cluster of functionally related genes, an operon (natural or synthetic) coding for any functions or pathways.

In another specific illustrative embodiment, the polynucleotide sequences of interest codes for protein of interest. For example, this protein is an endogenous protein or a protein which is not naturally expressed by the bacterial strain according to the invention, also referred to as a heterologous protein. Preferably, the protein of interest is a protein of industrial interest such as enzymes, such as proteases, lipases, amylases; hormones; antigens, for example, usable as immunogens, peptides or proteins for therapeutic use, for example antibiotics; the protein of interest can thus find application in the field of crop protection, vector control, the commercial production of enzymes and the pharmaceutical industry, in particular for the production of vaccines.

The polynucleotide molecule int has at least 80%, preferably at least 85%, 90%, 95% or 100% identity with the sequence of SEQ ID No 4 coding for ^{mv4}Int.

In a further embodiment, the ^{mv4}Int of SEQ ID No 5 is comprised in the kit instead of the polynucleotide molecule int.

The present invention further relates to a vector comprising polynucleotide molecule A and optionally polynucleotide molecule int. The polynucleotides molecules A and int may be inserted in the same vector.

Alternatively, the polynucleotide molecule A is inserted in a first vector and the polynucleotide molecule int is inserted in a second vector.

A vector refers to any means for the cloning of and/or transfer of a nucleic acid into a host cell. This insertion is realized with techniques known to those skilled in the art, such as cloning using restriction endonucleases and DNA ligases, or DNA assembly methods (Gibson et al., 2009, Zhu et al., 2007).

The present invention further relates to a method for integrating a polynucleotide sequence of interest into the genome of a bacterial host cell comprising:
a—preparing a vector comprising a polynucleotide molecule A comprising:
(i) a sequence of between 220 to 250 pb comprising the following polynucleotide fragments P1-P2, C—O—C′ and P′1-P′2 wherein:

P1-P2 is
(SEQ ID No 2)
5′-ATCAACTAGATTTTTAACTAGAA-3′;

C—O—C′ is the site-specific recombination polynucleotide molecule as defined with the method for preparing a site-specific recombination polynucleotide molecule; and

P′1-P′2 is
(SEQ ID No 3)
5′-TTTAACTAGAAAATAACTAGAA-3′;

(ii) at least one polynucleotide sequence of interest;
b—transforming said bacterial host cell with the vector obtained at step (a) and the polynucleotide molecule int of SEQ ID No 4 coding for ^{mv4}Int;
c—maintaining said transformed host cell under conditions that allow integration of said polynucleotide sequence of interest into the genome of the host cell.

In a particular embodiment, at least two polynucleotide sequences of interest are integrated.

The polynucleotides molecules A and int are inserted in the same vector or different vector with the techniques described previously.

The bacterium is transformed with one or two vectors within techniques known to those skilled in the art, such as the use of classical selective markers (antibiotic resistance, auxotrophic complementation . . . ).

The ^{mv4}Int allows the integration of the polynucleotide sequence of interest into the bacterial genome. The coding sequence of ^{mv4}Int is inserted in a vector according to the invention or present in the recipient bacterial cell due to a previous transformation.

The present invention relates to a genetically modified bacterial host cell obtained by the method described previously.

A genetically modified bacterial host cell means a bacterium harbouring a polynucleotide sequence of interest integrated into the genome by the method according to the invention.

The present invention relates to a method production of a protein of interest comprising the steps of
a. preparing a genetically modified host cell described previously wherein the polynucleotide sequence of interest codes for a protein of interest;
b. culturing said host cell; and
c. optionally, purifying said protein of interest.

The present invention further relates an isolated polynucleotide molecule of SEQ ID No 4 coding for ^{mv4}Int and to the isolated ^{mv4}Int of SEQ ID No 5.

The ^{mv4}Int site-specific recombination system can serve as a new tool for bacterial genome engineering that will allow specific and irreversible integration of large fragments of foreign DNA into “user-defined” chromosomal sites of any bacterial species.

### BRIEF DESCRIPTION OF THE DRAWINGS

FIG. 1. Redefinition of the ^{mv4}Int sequence. (A) ^{λ}Int and ^{mv4}Int structure. The ^{λ}Int structure is from Biswas et al., (Biswas et al., 2005) and ^{mv4}Int structure was modelized on Alphafold (Jumper et al., 2021a) by using the sequence described in 1995 (Dupont et al., 1995b) and the corrected sequence presented in this paper. The ^{mv4}Int-1995 structure possesses an unstructured arm-binding domain and lacks the canonical antiparallel β-sheet (red arrows in ^{λ}Int) that position the lysine (purple residue) into the catalytic domain. The new ^{mv4}Int structure presents a canonical three-stranded n-sheet arm-binding domain, and a canonical antiparallel β-sheet (red arrows). (B) Schematic representation of ^{mv4}Int and consequences of sequencing errors on ^{mv4}Int sequence. Deletions (black cross) of a C, a T and three A, the inversion of GT/TG and CG/GC (two-way arrow) are found. Black boxes indicate protein regions different from the published sequence. (C) Alignment of catalytic domains of ^{λ}Int, Cre, XerC, XerD, ^{HP1}Int, ^{mv4}Int-1995 (the original sequence published in 1995) and ^{mv4}Int-2022 (the resequenced protein). The 7 conserved residues of the YR catalytic domain are indicated in bold and with an asterisk. The lysine residue (K) was manually adjusted based on the alignment performed by Nunes-Düby et al. (Nunes-Duby et al., 1998). Letters in bold grey for ^{mv4}Int-1995 sequence correspond to the amino acid sequence obtained with incorrect DNA sequencing performed in 1995.

FIG. 2. Principle of the use of randomized libraries for the characterization of core regions of ^{mv4}Int/attP/attB. (A) Localization of libraries in the attP (green) or attB (orange) core regions. The published minimal attB site (Auvray et al., 1999b) is framed by the black box. The atypical P2 arm-type binding site is indicated by a black arrow and the dark vertical arrows indicate the strand exchange position on top and bottom strands of the 8-bp overlap sequence. The randomized region of each library is represented by “N” and nucleotides identical to the native sequences are symbolized by dashes. (B) Global strategy for the use of randomized libraries. The chromatograms and the sequence logos show the proportion of each nucleotide at each position. The consensus sequence indicated is arbitrary. A colour is associated with each nucleotide: blue, C; green, A; red, T; dark and yellow, G.

FIG. 3. In vitro characterization of the minimal attB size. (A) Sequences used in this study for the in vitro recombination assay. Nucleotides differing from the native sequence are indicated by red lower-case letter. (B) Effect of the size of attB on the recombination reaction. The recombination reaction contains 7.2 pmol of ^{mv4}Int, 40 μg of E. coli crude extract heated at 95° C., and the reaction was incubated 1 h30 at 42° C. The size of the attB used is indicated above each lane. The fluorescent attB fragment and the recombination product (I) are indicated on the gel. Lane -, reaction without ^{mv4}Int. (C) Results of attB Lib9×attP_{WT} in vitro recombination. The bases described outside of attBₘᵢₙ are represented in grey. The dark vertical arrows indicate the two cleavage sites surrounding the published 8-bp overlap region (Coddeville et al., 2014b). “O” indicates the overlap region and “N” represents the randomized positions. Each nucleotide is associated with a colour on the chromatogram: blue, C; black, G; green, A; red, T. (D) Results of attB Lib6×attP_{WT} in vitro recombination. (E) Results of attB Lib5×attP_{WT} in vitro recombination.

FIG. 4. Characterization of the attB and attP overlap regions. (A) Representation of the two expected results of attP×attB_{WT} recombination with a randomized nucleotide at the last position of the overlap region. If the first position is included in the strand exchange region (8-bp overlap), only the nucleotide complementary to attB_{WT} (i.e. T) will be recovered. If the first position is excluded from the strand exchange region (7-bp overlap), the permissive nucleotides will be observed in one of the two attL or attR hybrid sites. (B) Results of attB_{WT}×attP Lib1 in vitro recombination. The red circle highlights the randomized position before and after recombination. (C) Results of attP_{WT}×attB Lib8 in vitro recombination.

FIG. 5. Characterization of the constraints exerted at the overlap region. (A) Results of attB Lib7×attP_{WT} in vitro recombination. The dark vertical arrows indicate the two ^{mv4}Int cleavage sites surrounding the overlap region “O”. (B) Results of attB_{WT}×attP Lib2 in vitro recombination. (C) Results of attB Lib7 recombination against 3 different attP overlap sequences. (D) Results of attB Lib7×attP Lib2 in vitro recombination. (E) Effect of the overlap sequence on recombination activity. The four different pairs of attB/attP sharing the same overlap sequence are represented. Nucleotides differing from the WT sequence are indicated in bold. Lanes WT, attB_{WT}/attP_{WT} pair; 1, attB1/attP1 pair; 2, attB2/attP2 pair; 3, attB3/attP3 pair, (-), no ^{mv4}Int.

FIG. 6. NGS characterization of the nucleotide constraints surrounding the overlap region in attB and attP sites. (A) Results of attB Lib6×attP_{WT} in vitro recombination. The upper Sequence Logo shows the nucleotides distribution in attB Lib6 library before performing the in vitro recombination. The two attL Sequence Logos represent the nucleotides distribution observed from two independent recombination experiments. Numbering above nucleotides represents the position at the randomized sequence. For attB Lib6, only the seven nucleotides that belong to the attB site are indicated (position 4 to 10). NGS Read number used for the sequence logo construction is indicated (M=10⁶). (B) Results of attB Lib8×attP_{WT} in vitro recombination. (C) Results of attP Lib3×attB_{WT} in vitro recombination. (D) Results of attP Lib1×attB_{WT} in vitro recombination. (E) Organization of the 21-bp attBₘᵥ₄ site and the attPₘᵥ₄-core region.

FIG. 7. DNA sequence representativeness after in vitro recombination of randomized libraries. (A) Occurrence (read count) of each motif relative to its ranking distribution on raw NGS data. Each in vitro recombination experiment was performed twice. Abbreviations: rep1, experimental repetition 1; rep2, experimental repetition 2. B corresponds to attP_{WT}×attB Lib6 recombination, B′ to attP_{WT}×attB Lib8 recombination, C to attB_{WT}×attP Lib3 recombination, and C′ to attB_{WT}×attP Lib1 recombination. Horizontal dashed line represents cut-off at 1000 occurrence. Vertical dashed line corresponds to a cut-off for the 100 most represented motifs. The region up to 2000 occurrences and the 2000 most represented motifs is enlarged for better visibility. (B) Sequence Logos determined from the nucleotide sequences of the 100 most enriched motifs (determined as the ratio of occurrence after recombination/occurrence before recombination).

FIG. 8. ^{mv4}Int binding characteristics by EMSA experiments. (A) Effect of P′12 arm-type oligonucleotide on ^{mv4}Int binding to the core-attP region. A fluorescent fragment containing the 21-bp COC′ sequence (0.87 μmol) was incubated in the presence or absence of ^{mv4}Int (25 μmol) and in the presence or absence of unlabelled DNA containing P′12 arm binding sites (4.48 μmol). Reactions were analysed by native 7.5% PAGE and fluorescence was visualized on the Chemidoc MP Imaging system (Biorad). The presence or absence of ^{mv4}Int and arm-type sites (28 bp or 40 bp) is indicated above the gel. The different complexes are indicated on the gel: ss DNA, single-stranded DNA; ds DNA, double-stranded DNA; I, one monomer of ^{mv4}Int bound to the core region; II, one monomer of ^{mv4}Int bound to the core and arm region of 28 bp (II) or 40 bp (II*); III, dimer of ^{mv4}Int bound to the core and arm region of 28 bp (III) and 40 bp (III*). (B)^{mv4}Int binding to the COC′ region of attP. Legend is identical to A. The reaction contains 0 pmol (lane 1), 3.6 pmol (lane 3), 10.7 pmol (lane 4), 18 pmol (lane 5), 25.1 pmol (lanes 2 and 6) and 35.8 pmol (lane 7) of ^{mv4}Int. (C)^{mv4}Int binding to the BOB′ sequence. Legend is identical to B. (D)^{mv4}Int binding to the B′/C′ core-binding site. Legend is identical to B. (E)^{mv4}Int binding to the B core-binding site. Legend is identical to B. (F)^{mv4}Int binding to the C core-binding site. Legend is identical to B.

FIG. 9. ^{mv4}Int arm-binding sites characterization. (A) Characterization of the ^{mv4}Int arm-binding site by stabilization of ^{mv4}Int binding to the COC′ core sequence. A fluorescent fragment including the COC′ core sequence (0.87 μmol) was incubated in the presence of 4.48 pmol of unlabelled arm-binding sites (the arm-binding region is indicated above the lanes) and 25.1 pmol of ^{mv4}Int. Lanes: 0, no ^{mv4}Int; -, no arm-binding oligonucleotide. The three complexes are indicated and are identical to those determined previously. (B) Effect of size and relative orientation of the arm-binding sites on the stabilization of ^{mv4}Int binding to the COC′ core region. The legend is identical to A. (C) Schematic representation of the published attPₘᵥ₄ and attBₘᵥ₄ sites and comparison with the structure described in this study. Dark grey boxes indicate arm-binding sites, and their orientation is represented by arrows. Light grey triangles represent the excisionase binding sites (Coddeville and Ritzenthaler, 2010) and grey boxes represent the core region (region of identity between attB and attP).

FIG. 10. ^{mv4}Int mediates recombination towards other bacterial tRNA^{SER} sequences. (A) tRNA^{SER} sequences and the adapted attP core sequence used for the in vitro assay. The two ^{mv4}Int cleavage sites surrounding the overlap region (O) are indicated by the vertical black arrows. Nucleotides that differ from the attB_{WT} sequence are shown in bold. (B) Heterologous tRNA^{SER}sites able to recombine both attP_{WT} and the adapted attP. The recombination reaction contains 7.2 pmol of ^{mv4}Int and was incubated for 16 h at 42° C. Lanes: a, attP_{WT}×attB_{X} recombination; b, attP_{X}×attB_{X} recombination; c, b without ^{mv4}Int; “+” attP_{WT}×attB_{WT} recombination; “−” attP_{WT}×attB_{WT} recombination without ^{mv4}Int. The bacteria from which attB_{X} (tRNA^{SER}) comes from is indicated above the gels. Lanes: a, attP_{WT}×attB_{X} recombination; b, attP_{X}×attB_{X} recombination; c, b without ^{mv4}Int. (C) Sequences where recombination is only possible with the adapted attP. (D) Sequences where recombination is impaired. Lanes: a, attP_{WT}×attB_{X} recombination; b, attP_{X}×attB_{X} recombination; c, b without ^{mv4}Int. Nucleotides absent for the attB consensus sequence are shown in red.

FIG. 11. ^{mv4}Int-mediated site-specific integration in the chromosomal tRNA^{SER} from E. coli and L. lactis. (A) Nucleotide sequences of the 21-bp attB consensus, mv4 attB_{WT}, E. coli and L. lactis tRNA^{SER}. Nucleotides differing from attB_{WT} are indicated in bold. Nucleotide excluded from the consensus sequence is indicated in red. (B) Theoretical outcomes of the chromosomal integration into the E. coli or L. lactis tRNA^{SER}. Primers used for the PCR are indicated with black arrows and the size of each amplicon is indicated. (C) PCR amplification of E. coli or L. lactis attB sites, before (I) and after (II) integration (five integrants). Lane M, 1 kb DNA ladder (New England Biolabs). (D) PCR amplification of E. coli or L. lactis attL sites (I) from the five integrants used in (C). Lane M, 100 pb DNA ladder (New England Biolabs).

FIG. 12. In vitro reprogramming of the recombination to 3 putative sites present in E. coli lacZ gene. (A) Artificial attB sites and adapted attP used. The nucleotides different from the native attB_{WT} are indicated in bold. (B) Fluorescent in vitro assay against the 3 lacZ sites. Recombination reaction contains 7.2 pmol of ^{mv4}Int, 40 μg heated E. coli crude extract and was incubated 1 h 30 at 42° C. The attB/attP pair used is indicated above each lane. Fluorescent linear attB and recombination product (I) are indicated on the gel. Lane T, reaction without ^{mv4}Int. (C) PCR amplification of attL and attR for each attB/attP pair tested. Lane M, 100 bp DNA ladder (New England Biolabs). (D) Sanger sequencing of attL and attR recombination products. Nucleotides in grey are the nucleotides belonging to attP_{WT} and nucleotides that differ from attB_{WT} are indicated in bold.

FIG. 13. Effect of nucleotides excluded from BOB′ consensus sequence on in vitro recombination. A) Sequences of attB variants. Nucleotides not included in the consensus sequence are indicated in bold. (B) Fluorescent in vitro assay of recombination attP_{WT}×each attB variant. Recombination reaction contains 7.2 pmol of ^{mv4}Int, 40 μg heated E. coli crude extract and was incubated 1 h 30 at 42° C. Lanes: 1, attP_{WT}×attB_{WT}; 2, attP_{WT}×attB_{A5C5}; 3, attP_{WT}×attB_{A5T5}; 4, attP_{WT}×attB_{T7C7}; 5, attP_{WT}×attB_{T7G7}. Fluorescent linear attB and recombination product (I) are indicated on the gel. Lane T, reaction without ^{mv4}Int.

### DETAILED DESCRIPTION

### Examples

### Material and Methods

### Strains, Plasmids, Primers, and Media

The different strains and plasmids used in this study are listed in Table 5 and 6. All sequences of primers that were used are available in Table 7A, 7B, 7C and 7D. The E. coli strain NEB5-α repA+ was built by using the protocol from Datsenko and Warner (Datsenko and Wanner, 2000). It was constructed by replacing the glgB gene with the glgB::Kan-repA region from E. coli strain EC1000. E. coli strains were grown in Lysogenic Broth (LB) at 37° C. L. lactis were grown on GM17 at 28° C. Antibiotics were used at the following concentration: carbenicillin, 100 μg/ml; chloramphenicol, 12.5 μg/ml; erythromycin, 150 μg/ml (1 μg/ml for L. lactis); kanamycin, 50 μg/ml.

### DNA Procedures

Standard techniques were used for DNA manipulation and cloning. Polymerase chain reaction (PCR) was performed with Q5-HF polymerase (New England Biolabs) or with CloneAmp Hifi polymerase (Takara Bio), according to the manufacturer's instructions. PCR products were purified using the QIAquick PCR purification kit (Qiagen). Plasmids were constructed using Gibson assembly (42) with NEBuilder HIFI DNA Assembly (New England Biolabs) or blunt-end cloning with T4 PNK (New England Biolabs) and T4 DNA ligase (New England Biolabs), according to the manufacturer's instructions. Plasmid DNA was extracted using QIAprep Spin Miniprep kit (Qiagen) or Nucleobond Xtra Midi (Macherey-Nagel) and their sequence was verified by Sanger sequencing (Mix2seq, Eurofins).

Constructing a Randomized attB Library and Core-attP Library

The randomized oligonucleotides (109 bp, attB library; 184 bp, core-attP library) were obtained by chemical synthesis (IDT, USA). PCR was used to create double-stranded DNA using primers attBlibrary-F and attBlibrary-R for attB and attPlibrary-F and attPlibrary-R for attP (Table 7B). Each PCR product was separately cloned either into pCC1Fos (Lucigen, USA) for attB libraries, or plasmid pMET359 (Table 6) for attP libraries by DNA assembly (Gibson et al., 2009). Clones were propagated in E. coli EP1300 (Lucigen, USA) under chloramphenicol selection for attB libraries and NEB5-α repA+(Table 5) under carbenicillin selection for attP libraries.

Purification of ^{mv4}Int

For ^{mv4}Int purification, the pET-Int plasmid (Table 6) was transferred into E. coli strain BL21(DE3) (New England Biolabs). The resulting strain was grown in LB at 42° C. up to an OD600 of 0.6. Integrase gene expression was induced by addition of 0.1 mM of IPTG, and the culture was incubated at 22° C. for 3 h. Cells were recovered by centrifugation, resuspended in buffer A (50 mM Tris pH 8, 500 mM NaCl, 20 mM imidazole, 10% glycerol, 1 mg/ml lysozyme, and one tablet of SIGMAFAST Protease Inhibitor Cocktail Tablets EDTA-Free [Merck, Germany]), and disrupted by sonication (10 cycles of 30 sec at 40% intensity in ice, followed by 45 sec of rest between each cycle). The lysate was cleared by centrifugation (20000 g, 4° C., 20 min). ^{mv4}Int was first purified on nickel-nitrilotriacetic acid affinity resin (1 ml His-trap HP, GE Healthcare). Column equilibration was performed by injecting 10 column volumes of buffer B (50 mM Tris pH 8, 500 mM NaCl, 20 mM imidazole, 10% glycerol). After equilibration, the lysate was injected and unbound protein were washed using 10 column volumes of buffer B. ^{mv4}Int was eluted using a buffer C gradient of 0 to 30% (50 mM Tris pH 8, 500 mM NaCl, 500 mM imidazole, 10% glycerol). Eluted fractions were then injected in a gel filtration column (HiLoad 16/60 Superdex 200, GE Healthcare, USA). This column was equilibrated using 2 column volumes of buffer D (50 mM Tris pH 8, 500 mM NaCl, 10% glycerol, 1 mM DTT, 1 mM EDTA) and the fractions containing ^{mv4}Int were injected and eluted using the same buffer. Eluted fractions containing ^{mv4}Int were then 2-fold diluted in buffer E (50 mM Tris pH 8, 10% glycerol, 1 mM DTT, 1 mM EDTA). A heparin column (1 ml HiTrap Heparin HP, GE Healthcare, USA) was equilibrated using 10 column volumes of buffer F (50 mM Tris pH 8, 250 mM NaCl, 20% glycerol, 1 mM DTT, 1 mM EDTA). Eluted fractions containing ^{mv4}Int were then injected and unbound protein were removed using 10 column volumes of buffer F. ^{mv4}Int was eluted using a buffer G gradient of 0 to 100% (50 mM Tris pH 8, 1 M NaCl, 20% glycerol, 1 mM DTT, 1 mM EDTA). Purified integrase was aliquoted, snap-frozen in liquid N₂ and stored at −80° C. in buffer containing 50 mM Tris pH 8, 500 mM NaCl, 20% glycerol, 1 mM DTT, 1 mM EDTA.

### In Vitro Fluorescent Assay

Reaction mixtures (20 μl) contained 0.08 pmol (300 ng) of supercoiled plasmid carrying the attP site, 0.08 pmol (15 ng) of linear fluorescent (Cy3) 308-bp attB fragment, 7.2 pmol (300 ng) of ^{mv4}Int and 40 μg of a crude-extract from E. coli BL21(DE3) heated at 95° C. for 10 min, in 25 mM Tris pH 7.5, 1 mM EDTA, 150 mM NaCl, 1 mM DTT, and 10% PEG8000 (TENDP 1× buffer). The reaction was incubated at 42° C. either 1 h 30 or 16 h and was stopped by addition of 0.1% SDS. Samples were analysed by electrophoresis in 0.8% agarose gels. Fluorescence was revealed using the ChemidocMP imaging system (Biorad).

### In Vitro Recombination Assay Using Libraries

Reaction (20 μl) containing 0.08 pmol (450 ng) of attB plasmid, 0.08 pmol of attP plasmid, 7.2 pmol of ^{mv4}Int and 40 μg of crude-extract from E. coli BL21(DE3), heated at 95° C. for 10 min, in TENDP 1× buffer (25 mM Tris pH 7.5, 1 mM EDTA, 150 mM NaCl, 1 mM DTT, and 10% PEG8000) and incubated 1 h 30 at 42° C. The attB, attP, attL and attR sites were amplified by PCR using SeqbanqueattB-F/SeqbanqueattB-R (Table 7B) primers for attB; SeqbanqueattP-F/SeqbanqueattP-R (Table 7B) primers for attP; SeqbanqueattB-F/SeqbanqueattL-R (Table 7B) primers for attL and SeqbanqueattR-F/seqbanqueattP-R (Table 7B) primers for attR. PCR products were purified and analysed by Sanger sequencing (Mix2seq, Eurofins).

### NGS Sequencing

PCR products (attL from recombination attB Lib6×attPWT; attBWT×attP Lib1 and attR from recombination attBWT×attP Lib3; attB Lib8×attPWT) used for Sanger sequencing were also used for NGS sequencing (Eurofins). Data were uploaded on the public server at usegalaxy.org (Afgan et al., 2018) for various analyses. Sequence Logo were generated using Weblogo3 (Crooks et al., 2004) and the occurrence of each word was characterized using the Wordcount program (Rice et al., 2000).

### Electrophoretic Mobility Shift Assay

5′ Cy3 end-labelled synthetic oligonucleotides (HPLC purified) were obtained from Eurofins. Labelled double strand DNA substrates were prepared by hybridization of complementary oligonucleotides (Table 7C) in 10 mM Tris pH 7.5, 50 mM NaCl by incubating the samples 5 min at 95° C. in a thermal cycler (Biorad) and decreasing the temperature of 1.5° C./min until it reaches 25° C. Binding reactions (20 μl) were performed with 0.87 pmol of labelled core- or arm-type DNA and 4.48 pmol of unlabelled arm- or core-type DNA in buffer containing 25 mM Tris pH 8, 75 mM NaCl, 10% glycerol, 0.5 mM DTT, 0.5 mM EDTA, 1 μg polydIdC (Sigma), 0.1 mg/ml BSA. The protein was added, the reaction performed at room temperature for 20 min and samples were loaded onto a non-denaturing 7.5% polyacrylamide gel (Mini-PROTEAN TGX, Biorad). The gels were run at 4° C., 75V for 2 h. Fluorescence was revealed using the ChemidocMP imaging system (Biorad).

### In Vivo Recombination

L. lactis strain MG1363 was transformed as described by Le Bourgeois et al., (Le Bourgeois et al., 2000) by using 1 μg of plasmid pMET306 (Table 6). Cells were incubated 3 h at 28° C. and selected for erythromycin resistance on M17 plates supplemented with 5 g/L of glucose. For E. coli, commercially electrocompetent EPI300 cells (Lucigen) were used and transformed with 300 ng of plasmid pMET376 (Table 6). Cells were incubated for 5 h at 37° C. and selected for carbenicillin resistance on LB agar plates. Genomic DNA of antibiotic resistant cells was extracted using the DNeasy Blood and Tissue kit (Qiagen). Site-specific recombination into the targeted tRNA^{SER}(CGA) was verified by amplifying the attB and attL sites by PCR. PCR amplification was performed using 1 ng of genomic DNA in 25 μL of 1×Q5 buffer (New England Biolabs), containing 800 μM of dNTP, 0.5 U of Q5 polymerase (New England Biolabs) and 0.5 μM of each primer. For attB amplification, the thermal cycle program consisted of a 5 min denaturation period at 98° C., followed by 30 cycles of a three-steps thermal profile (10 s at 98° C., 30 s at 60° C., and 3 min at 72° C.) ended with one cycle at 72° C. for 2 min. For attL amplification, the thermal cycle program consisted of a 5 min denaturation period at 98° C., followed by 30 cycles of a three-steps thermal profile (10 s at 98° C., 30 s at 60° C., and 30 s at 72° C.) ended with one cycle at 72° C. for 2 min. PCR products were analysed after electrophoresis in 0.8% agarose.

### Results

The ^{mv4}Int is a 369-Aminoacids Tyrosine Integrase

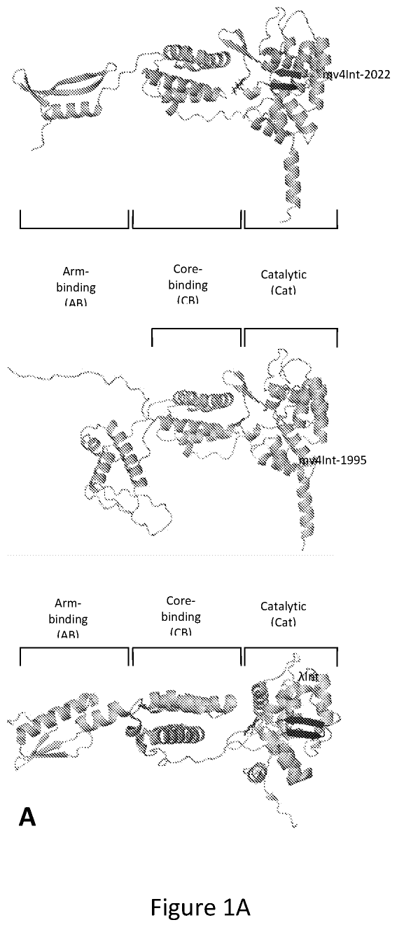

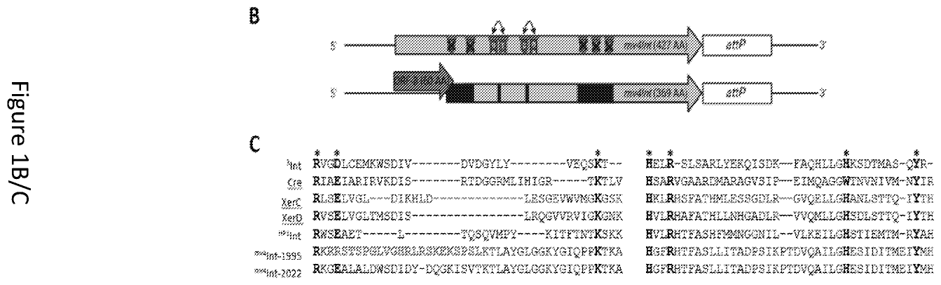

The original analysis of the integration region of mv4 bacteriophage described the ^{mv4}Int as a 427-aminoacids (AA) protein with significant similarity with the ^{λ}Int integrase (Dupont et al., 1995a). This result was confirmed through its comparison with other Y recombinases (Nunes-Diby et al., 1998), although ^{mv4}Int contains only six from the seven conserved residues defining the Int family of SSR, with the structurally important D215 residue of ^{λ}Int (E176 in P1 Cre) missing (FIG. 1C). Moreover, when ^{mv4}Int-427-AA was subjected to protein structure prediction program (Jumper et al., 2021b), discrepancies were observed when compared to ^{λ}Int, such as the lack of the two ß-strands surrounding the catalytic K235 residue, and absence of a structured AB domain (FIG. 1A). The ^{mv4}int gene from the pMC1 plasmid (Dupont et al., 1995a) was then resequenced and revealed five single nucleotide deletions and two inversions (FIG. 1B) compared to the published sequence. These nucleotides modifications were also observed from the different ^{mv4}int gene-containing plasmids that have been published previously, p3Aint and pET-Int (Auvray et al., 1999a), as well as from a PCR amplicon of the int region from the bacteriophage mv4 (data not shown). The ^{mv4}Int amino acid sequence is deeply impacted by these variations (FIG. 1B), since ^{mv4}Int corresponds to a 369-aminoacids protein that differs from the published sequence by fifty-three residues. Comparison of its catalytic domain (residues 172 to 369) against λInt, Cre, XerC, XerD and ^{HP1}Int recombinases (FIG. 1C) indicates that ^{mv4}Int-369-AA contains the 7 conserved residues R-D/E-K-H-R-H/W-Y of the tyrosine recombinases (YR) catalytic pocket (Gibb et al., 2010). In addition, the new ^{mv4}Int predicted structure (FIG. 1A) reveals better similarity with the three different domains of the ^{λ}Int monomer (Wojciak et al., 2002; Aihara et al., 2003). The native form of the ^{mv4}Int protein was overproduced, purified, and used for in vitro recombination assays (see Materials and Methods) between an attP site located on a supercoiled plasmid pMC1, (Dupont et al., 1995a) and a 308-pb PCR amplicon of the L. bulgaricus attB region. The Inventors demonstrated that the 369-AA ^{mv4}Int alone, i.e. without any accessory protein, was sufficient to catalyse site-specific recombination, and that reaction was abolished when using variants of each of two important residues of the YR catalytic site (Gibb et al., 2010), Y349F or K248A (data not shown, FIG. 1C).

### Global Strategy of the Use of Randomized Libraries

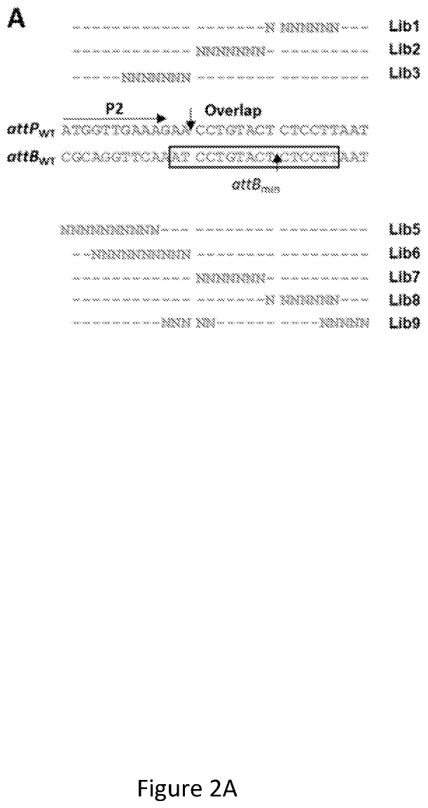

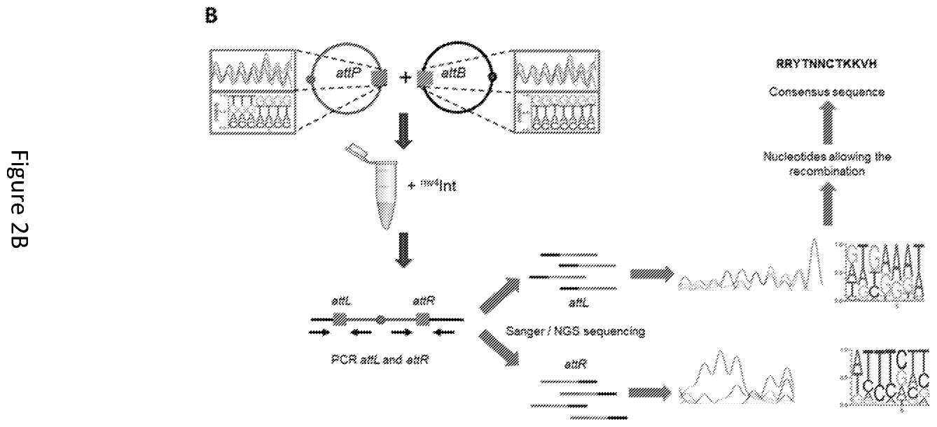

Due to the originality of the core region of attP and of the attB site, these regions were reanalysed by an approach based on the use of randomized DNA libraries. Those DNA libraries, corresponding either to the attB site or to the core-attP region, contained 7 to 10 randomized positions (FIG. 2A). Oligonucleotides containing at precise positions the 4 possible nucleotides were synthetized, amplified by PCR, and cloned in E. coli into pCC1Fos or pMET359 (see Materials and Methods). Each plasmid library was recovered, verified by Sanger sequencing, and used for in vitro recombination experiments (FIG. 2B) with either the native partner site (attP_{WT} or attB_{WT}) or the cognate partner library (same randomized region for the 2 sites). After recombination, attL and attR sites were amplified by PCR and sequenced. Among the randomized positions, only the nucleotides allowing the recombination should be recovered in these hybrid sites, which will make it possible to determine the constraints exerted on the nature of the nucleotide at each position tested (FIG. 2B).

Redefining the Minimal attB_{Mv4} Site

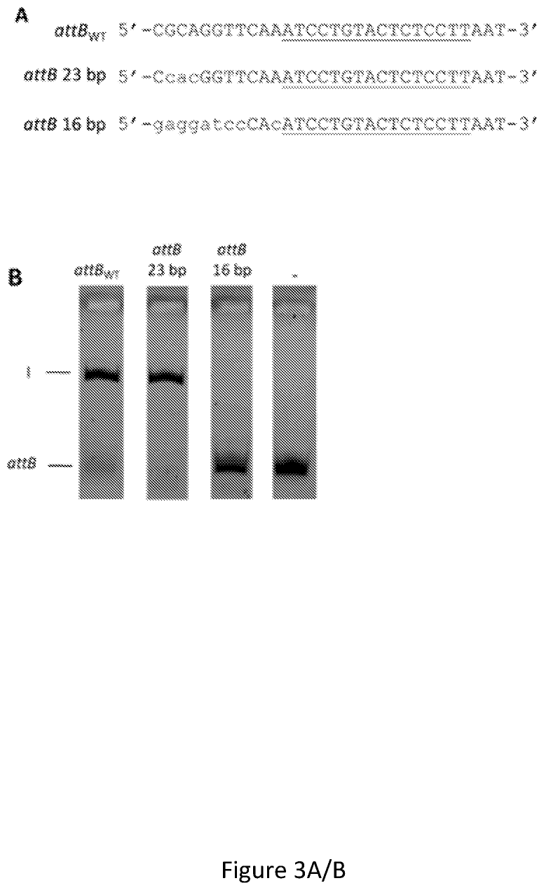

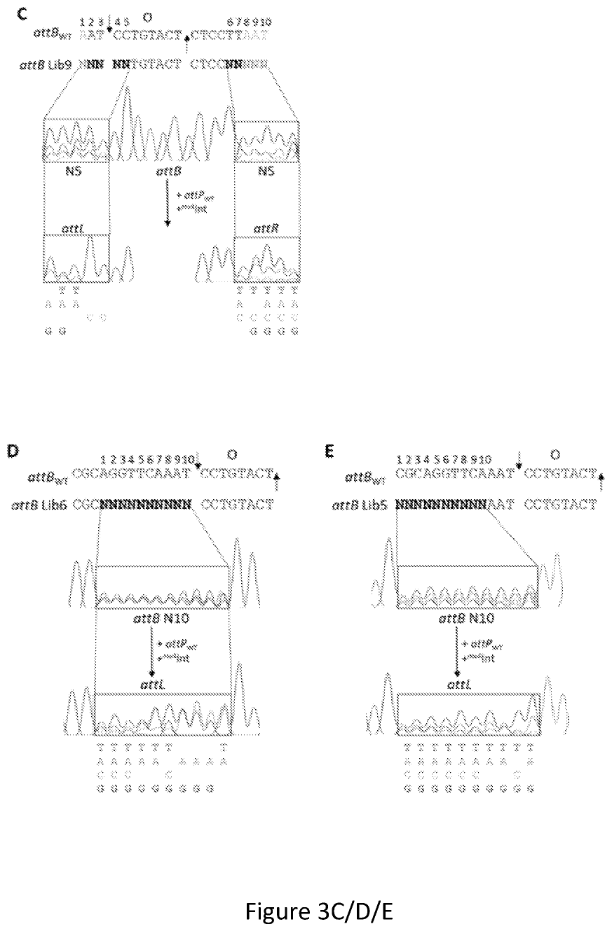

The attB minimal site has been previously characterized by Auvray et al., and resulted in a 16-bp sequence, the shortest attB described in the literature (Auvray et al., 1999b). In order to validate the particular size of this site, different sizes of attB_{WT} were amplified by PCR (FIG. 3A) to obtain fluorescent attB fragments, which were tested in our in vitro recombination assay against a plasmid-borne attP_{WT} site (FIG. 3B). A recombination product was obtained with the attB_{WT} region and with the 23 bp attB site but not with the 16 bp site indicating that the published site is not functional and that attB requires a longer sequence on its left side. To precisely characterise the boundaries of attB site, a library composed of five randomized positions overlapping each end of the published attB site was constructed (attB Lib9, FIG. 2A) and tested by in vitro recombination against the attP_{WT} site. After recombination, attL and attR sites were amplified by PCR and analysed by Sanger sequencing (FIG. 3C). On the right side of attB, the attR site displays several nucleotides at every randomized position, except for positions 6 and 7 that lack G and A, respectively, indicating that the attB site ends with the sequence 5′-CTCCTT-3′, in agreement with the previous study (Auvray et al., 1999b). On the left side, strong constraints are observed on attL since only one C is recovered at positions 4 and 5 after recombination. These positions correspond to the left end of the overlap region (Coddeville et al., 2014a) where nucleotides must be identical between the attB and attP sites, as observed for most recombination systems mediated by tyrosine-recombinases. Constraints are also observed at positions 1, 2, and 3 of the randomized region since only purines were detected, thought it was a position previously described outside of the minimal attB site (Auvray et al., 1999b). To precisely locate the left end of the attB site, two additional random libraries (Lib 5 and Lib6, FIG. 2A) were constructed and tested in vitro against the attP_{WT} site (FIG. 3D-E). Sequencing of the attL site from attB Lib6×attP_{WT} recombination indicates that positions 1 to 3 did not revealed any constraints (FIG. 3D), suggesting their location outside attB. This left border was also validated by the sequencing of the attL site from attB Lib5×attP_{WT} recombination (FIG. 3E), which confirmed the absence of constraints at the same positions. Altogether, results strongly suggest that the attB site of the mv4 recombination system corresponds to a site of 21-bp in length of sequence 5′-TTCAAATCCTGTACTCTCCTT-3′ (SEQ ID No 6).

Redefining the Size of the attB and attP Overlap Regions

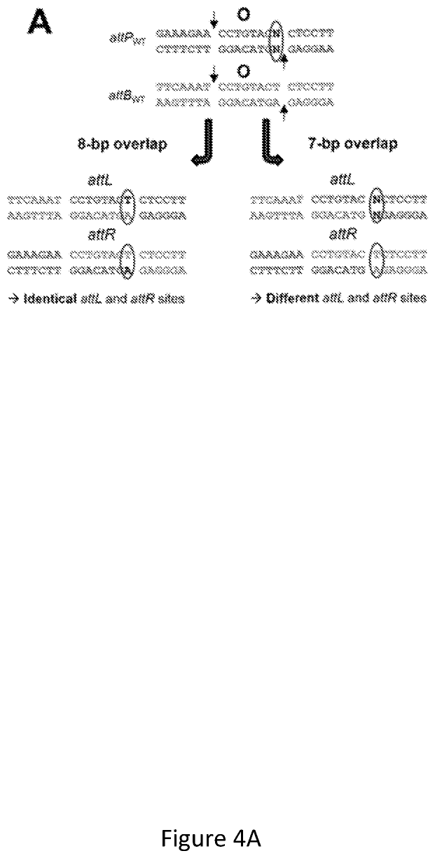

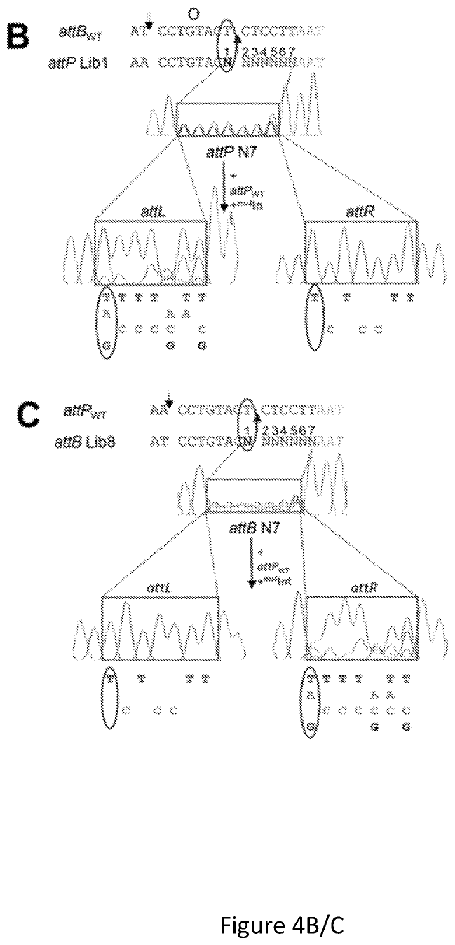

A previous study based on the use of DNA suicide substrates determined the length of the attBₘᵥ₄ and attPₘᵥ₄ overlap regions to 8 bp (Coddeville et al., 2014a), a size-range typical of YR recombination systems (Grindley et al., 2006). However, the Inventors noticed that all heterobivalent recombination systems currently characterised, such as phages λ (Craig and Nash, 1983), HK022 (Kolot and Yagil, 1994), HP1 (Hauser and Scocca, 1992), L5 (Peña et al., 1996), P22 (Smith-Mungo et al., 1994) or the ICE CTnDOT (Malanowska et al., 2006), display a 7-bp overlap region. As the knowledge of the strand exchange mechanism used by tyrosine recombinases make it possible to determine indirectly the length of the overlap region (Grindley et al., 2006), it has been taken advantage of this property to reanalyse the overlap region of the ^{mv4}Int/attP/attB system using random DNA libraries. Indeed, as the two overlap regions of attP/attB from phage systems must contain identical DNA sequence to promote a full recombination reaction, only one of the four nucleotides present on the attP or attB random libraries should be recovered on both attL and attR sites if the position is included into the overlap region (FIG. 4A). In contrast, if this position is located outside the overlap region, all permissive nucleotides should be recovered at one of the recombined sites, attL or attR depending on if the random library corresponds to attP or attB sites. As the left border of the overlap region was already verified with the use of the random library attB Lib9 (FIG. 3C), its right border was determined by the use of two different libraries (attB Lib8 and attP Lib1, FIG. 2A) tested by in vitro recombination against their cognate site, attP_{WT} or attB_{WT}, respectively. In both cases, one of the two recombined sites (attL or attL) contained three nucleotides at positions 1 of the random libraries (FIG. 4BC), demonstrating that the overlap regions of attP and attB is indeed of 7-bp in length instead of the 8-pb determined previously (Coddeville et al., 2014a).

Characterization of the Nucleotide Constraints Existing on attB and attP Overlap Regions

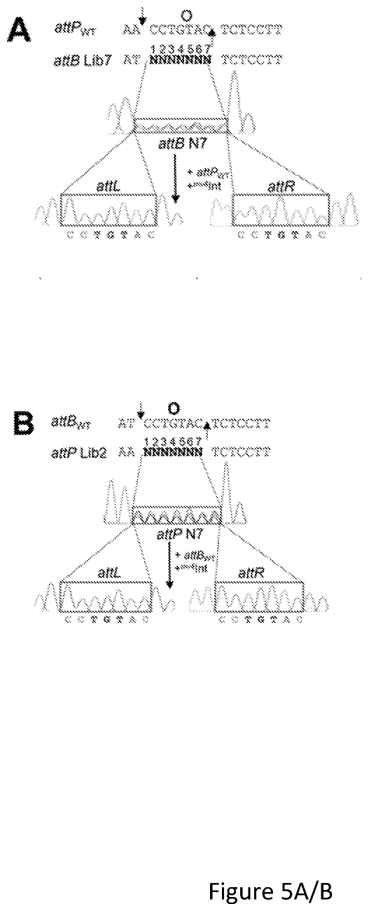

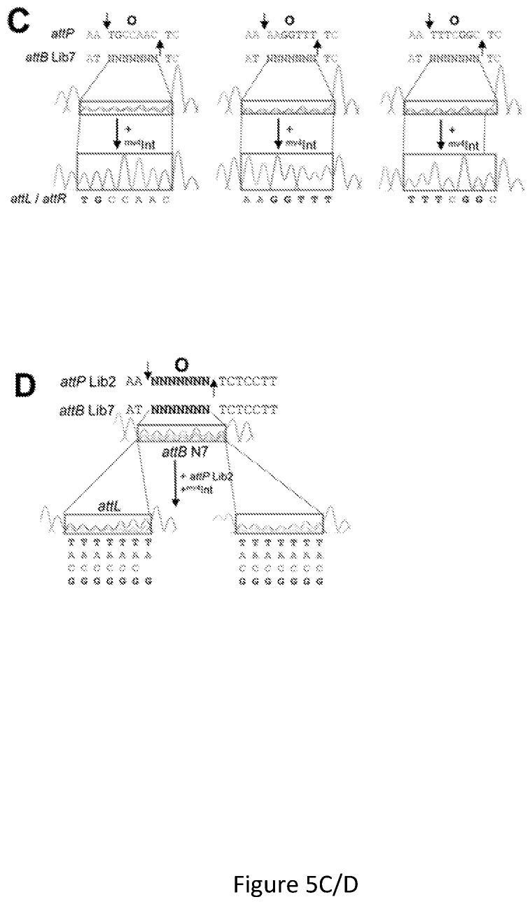

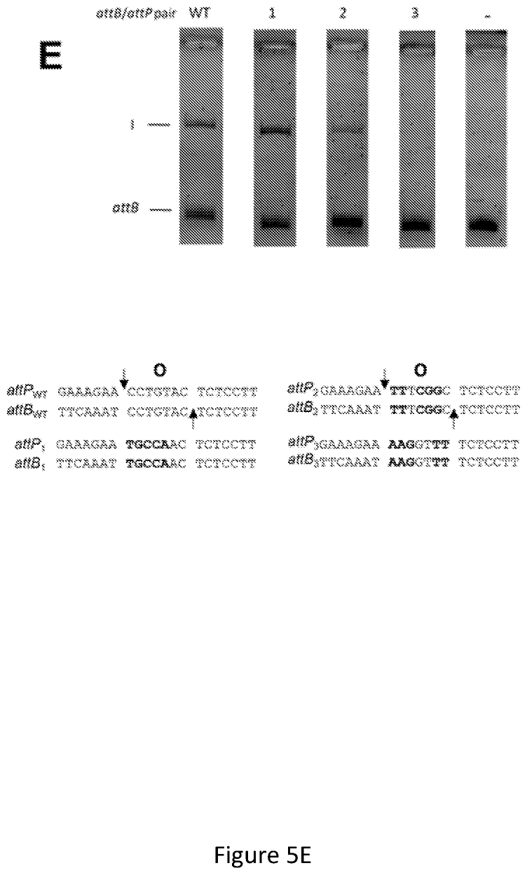

For the model integrase ^{λ}Int, it has been soon observed that the nature of the bases in the overlap region was not important for recombination but that sequence identity between attB and attP overlap was mandatory (Weisberg et al., 1983; Bauer et al., 1985). A similar feature has been observed for the phage HK022 recombination module (Kolot et al., 2015) and for Flp (McLeod et al., 1986) and Cre (Hoess et al., 1986), although it has been shown that presence of heteroduplex in the overlap of loxP sites can sometimes be functional (Lee and Saito, 1998; Sheren et al., 2007). In order to characterize the constraints exerted in the overlap region, two libraries composed of 7 randomized positions in the overlap region were constructed (attB Lib7 and attP Lib2, FIG. 2A) and tested in vitro against attP_{WT} (FIG. 5A) or attB_{WT} (FIG. 5B), respectively. After recombination, among the 16,384 (4⁷) starting sequences, only the sequence corresponding to the WT overlap sequence (5′-CCTGTAC-3′) was recovered on attL and attR sites, whatever the library used (attP Lib2 or attB Lib7). In addition, the same attB Lib7 library was tested against three different attP overlap sequences (FIG. 5C). After recombination, only the sequence corresponding to the attP overlap region tested was recovered, though an adenine could be found in addition to the expected guanine at the 6^{th} position of the 5′-TTTCGGC-3′ overlap sequence (FIG. 5C, right), suggesting that some heteroduplexes might happen to some extent. Lastly, in vitro recombination between attB Lib7 and attP Lib2 libraries revealed that any nucleotide can be recovered, except perhaps for the seventh position where no peak of C could be observed in the chromatogram (FIG. 5D). Altogether, these results indicate that almost no constraint appears to exist into the attB and attP overlap regions at the level of nucleotide composition as long as these two regions remain identical. However, the in vitro recombination efficiency seems to depend on the nature of the overlap sequences because differences in fluorescence intensities of the recombined product, from a 2-fold (FIG. 5E, lane 2) to a 15-fold (FIG. 5E, lane 3) decrease compared to the WT overlap region, can be observed in our in vitro assays.

Characterization of the Nucleotide Constraints Exerted on the DNA Regions Surrounding the Overlap Sequence of attB Site and Core-attP Region

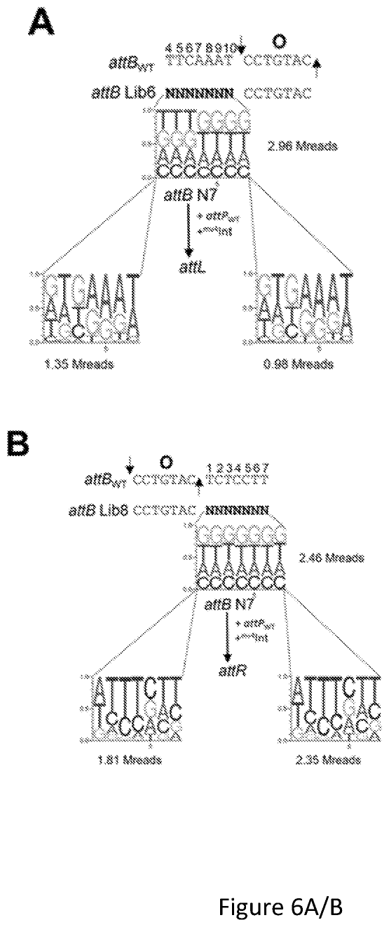

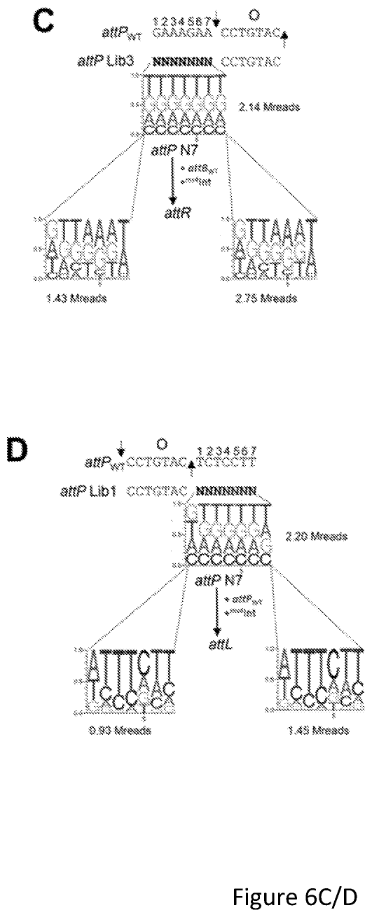

The attB Lib6 library (FIG. 2A) that allowed us to define a 21-bp attB site also allowed the analysis of the nucleotide constraints exerted on the left side of the attB overlap sequence. After recombination against the attP_{WT} site, Sanger sequencing revealed a 7-bp degenerated pattern of the attL site (FIG. 3D), with no C at positions 4 and 5, no A in position 6, only purines at positions 7, 8 and 9, and only A or T at position 10. Similar constraints were observed at the corresponding positions when analysing the attL site from the recombinations attP_{WT}×attB Lib9 (FIG. 3C) and attP_{WT}×attB Lib5 (FIG. 3E). These results strongly indicate that ^{mv4}Int-mediated recombination supports nucleotide variations of the attB site to some extend at its 7-bp left region, when recombined against the native attP site, defining a 7-pb degenerated pattern 5′-DDBRRRW-3′. However, when applied on a DNA population such as randomized libraries, the Sanger sequencing method is only resolutive enough to reveal prevalent nucleotides at each position. The nucleotide constraints of attB site and core-attP region were thus analysed through NGS sequencing (Illumina) of the recombined attL or attR sites (depending on the randomized region), allowing the individual sequencing of every molecule amongst millions of DNA fragments (Table 8) and the construction of Sequence Logos (Schneider and Stephens, 1990). In order to consider any biased nucleotide distribution during the construction of the four randomised DNA libraries used for these experiments (attB Lib6, attB Lib8, attP Lib3, and attP Lib1, FIG. 2A), PCR-amplified attB and attP sites were sequenced by NGS and their respective Sequence Logos constructed (FIG. 6).

Each library has the tendency to contain A or C slightly underrepresented compared to T and G, with a minimum of 15% of C and 20% of A for attP Lib3 library (FIG. 6D). The randomized region of the attL site from attB Lib6×attP_{WT} in vitro recombination was then reanalysed by NGS sequencing, and its Sequence Logo (FIG. 6A) strongly confirmed the 7-pb consensus pattern 5′-DDBRRRW-3′ determined by Sanger sequencing, though additional nucleotides appeared at low frequencies (see for instance the T observed at two of the three purines, FIG. 6A). In a similar manner, the right-side of the attB overlap region was analysed by performing Sanger (data not shown) and NGS sequencing (FIG. 6B) of the attR site from attB Lib 8×attP_{WT} recombination. Interestingly, the Sequence Logo revealed a 7-bp consensus pattern 5′-DYYYVHB-3′ complementary to the left side of the attB overlap region, though more degenerated (see for instance the C/G at position 6 or the G/A at position 7). The symmetry in nucleotide composition between the left and the right sides of the attB site suggests the presence of an imperfect inverted-repeated pattern surrounding its overlap region, an organization more characteristic of the recombination sites associated to tyrosine recombinases. Similar analyses were performed on the core-attP region using randomized DNA libraries surrounding the overlap region, attP Lib3 (FIG. 2A) for the left side of the O region and attP Lib1 (FIG. 2A) for its right side. After recombination against attB_{WT}, NGS sequencing of the randomized attR or attL regions, respectively, revealed two 7-bp symmetrical degenerated patterns, 5′-DDBDRDW-3′ at the left side of attP (FIG. 6C) and 5′-DYYYVHY-3′ at its right side (FIG. 6D), almost identical to their attB counterparts. In conclusion, the use of appropriate randomized DNA libraries covering the 21-pb of the core-attP region and attB site highlighted the classical organization of recombination sites of the mv4 system, with two inverted-repeat sequences (B and C for the left sides of attB and attP, and B′ and C′ for the right sides of attP, according to the lambda's recombination sites terminology) corresponding to putative core-binding sites of the integrase surrounding the strand-exchange region (the overlap region) of 7-bp (FIG. 6E).

Characterization of the B, B′, C and C′ Motifs Most Permissive for ^{mv4}Int-Mediated In Vitro Recombination

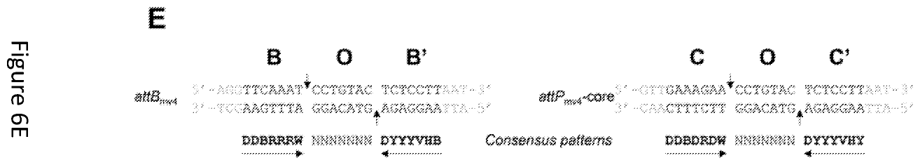

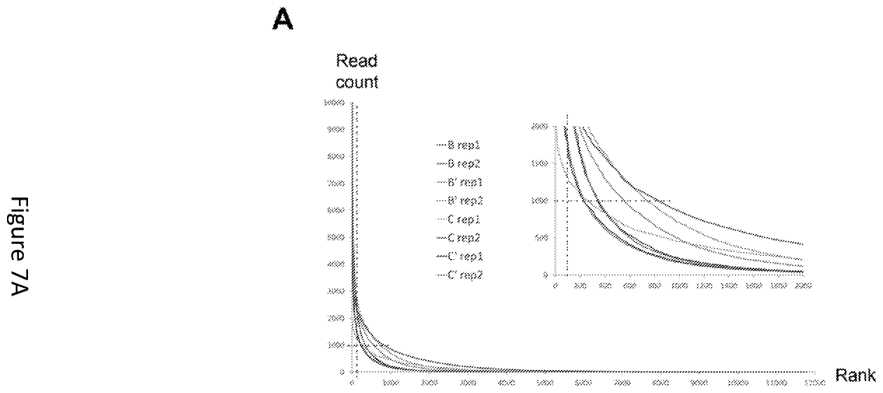

By determining the individual sequence of each molecule from a DNA population, NGS sequencing not only allows high resolution in the nucleotide composition of the randomized part of the libraries, but also to determine the occurrence of each 7-bp motif into the recombined DNA population. As it is highly unlikely that the thousands of motifs from the libraries will all be functional because of uncharacterized constraints, the Inventors assumed that comparing motifs occurrence in the randomized libraries before and after recombination by NGS sequencing will give relevant information about their ability to form a productive recombination complex with the mv4 integrase. Once constructed, each attB or attP library was sequenced and found to contains from 16312 to 16384 motifs (Table 8), corresponding from 99.5 to 100% of the theoretical number for 7-bp random library (4⁷). Depending on the library and the experimental repetition, one-third (33.32%) to two-thirds (71.91%) of the motifs were recovered after ^{mv4}Int-mediated in vitro recombination (Table 8). In addition, their occurrence was highly biased, with a factor ranging from 2,000 to 70,000, depending on the experiment, between the least and the most represented motif (Table 8), with a rapid drop in read counts relative to the rank (FIG. 7A). For example, from the several thousands of motifs recovered after recombination, only 250 to 850 have an occurrence of at least 1,000 (FIG. 6A), and fifty percent of the read counts are represented by only 167 (2.3%) to 784 (7.5%) of the recovered motifs (Table 8), depending on the library. In order to determine the B, B′, C, and C′ sites most permissive for recombination, occurrence of each motif from attL (B and C′ sites, FIG. 6A-D) or attR (B′ and C sites, FIG. 6B-C) hybrid site was divided by its occurrence before the recombination (attP or attB libraries), allowing to calculate an enrichment factor, with the hypothesis that the more a motif is enriched, the more it is permissive to recombination. However, as most of the motifs have a very low occurrence (for example, 30% to 50% of the motifs have an occurrence lower than 10) and are thus not representative of the NGS read population, the enrichment determination was only performed on the 250 to 850 motifs with an occurrence of at least 1,000. At last, only the 100 most frequent motifs were considered for the ranking. These motifs correspond from 10% to 35% of the total counts of the recovered motifs (FIG. 7A). Altogether, these results strongly indicate that amongst the thousands of functional sequences, only a small percentage allows recombination at a significant efficiency. Performing Sequence Logos on these 100 most enriched motifs not only confirmed the 7-bp consensus patterns previously obtained (FIG. 6E), as seen for B site (5′-DDBRRRW-3′), but reduced the degeneracy to some extend for sites B′ (5′-WYYYVHB-3′ instead of 5′-DYYYVHB-3′), C (5′-DDKDRDW-3′ instead of 5′-DDBDRDW-3′), and C′ (5′-WYYYMHY-3′ instead of 5′-DYYYVHY-3′) (FIG. 7B). One should note that all natural sites belong to these reduced degeneracy, except the C site for which the A in third position is excluded from the consensus. Remarkably, the natural motifs are not necessary the most enriched sequences after recombination (Tables 1-4), since B, B′, C, and C′ sites are ranked 40^{th}, 411^{th}, 253^{rd} and 16^{th}, respectively. This suggest that either other motifs can recombine more efficiently than the natural sites, or that natural sites are the best adapted sites for in vivo but not in vitro ^{mv4}Int-mediated recombination.

### B, B′, C and C′ Sites are the Core-Binding Sites for the Mv4 Integrase

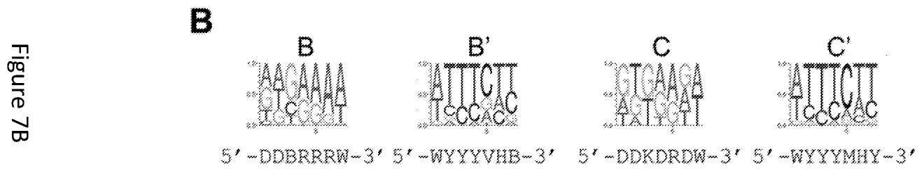

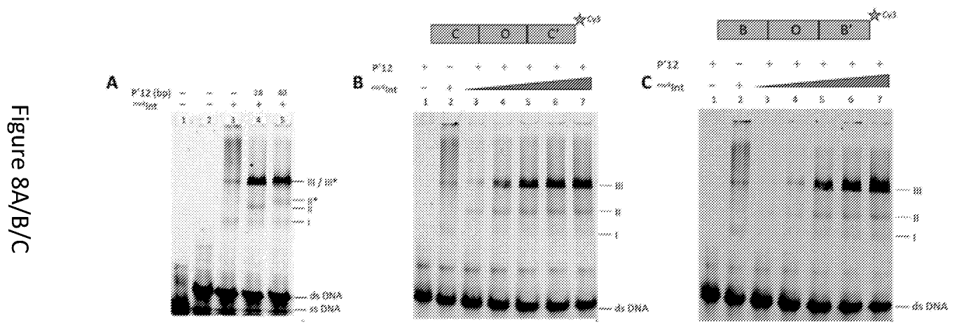

To experimentally demonstrate that imperfect inverted-repeats B, B′, C and C′ correspond to the ^{mv4}Int core-binding sites, the Inventors used a gel shift assay (EMSA) based on the protocol developed on the COC′ region of ^{λ}Int recombination system (Sarkar et al., 2001). In this study, Sarkar et al., demonstrated that the N-ter domain exerts an inhibitory effect on the C-ter domain when not bound to the P arm-binding sites. However, if DNA containing the P′1-P′2 arm-binding sites was added to the reaction, the inhibition was removed and ^{λ}Int was able to stably bind to the core-binding sites. To determine if ^{mv4}Int N-ter domain also exerts such inhibitory effect on its C-ter domain, they compared the ^{mv4}Int binding to a labelled 35-bp dsDNA containing the 21-bp core-attP region (Table 7C), in the presence or absence of unlabelled dsDNA containing the P′1 and P′2 arm-type binding sites (Table 7C). ^{mv4}Int binding to the COC′ sequence appeared quite unstable, as only faint bands and strong background smear can be observed (lane 3, FIG. 8A). Addition of a 28-bp dsDNA containing the P′12 sequences (Table 7C) greatly stimulates and stabilizes ^{mv4}Int binding to the core-attP sequence and led to the formation of three complexes (lane 4, FIG. 8A). In accordance with results from other YR systems, our data suggests that complex I should consist of a single ^{mv4}Int monomer bound to one core-binding site, whereas complex II should consist of a single ^{mv4}Int monomer bound to one arm and one core-binding site, and that complex III should correspond to a dimer of ^{mv4}Int bound to arm- and core-binding sites. To ensure that the stabilization effect was due to the binding of the N-ter domain of ^{mv4}Int to the arm-type binding site, the 28-pb P′12 dsDNA was replaced by a DNA duplex of 40 pb containing the P′1 and P′2 sites (Table 7C). A stabilization effect on the binding to the COC′ sequence was observed (lane 5, FIG. 8A), with the formation of two complexes (II* and III*) of mobility lower than the complexes observed with the 28-bp P′12 fragment. EMSA experiments clearly showed that ^{mv4}Int stably binds to both attB and core-attP sites, with an increase of complex III band intensity when increasing the ^{mv4}Int concentration (FIG. 8BC). As the left and right half-sites of attB and core-attP are not perfect inverted repeats, a study of the ^{mv4}Int binding to each half-site was performed using 35-bp dsDNAs mutated for either B′, C, or C′ sites (Table 7C). In all cases, a fluorescence signal weaker than for DNA fragments containing two binding sites (BOB′ or COC′) was observed (FIG. 8DEF), suggesting strong cooperativity between ^{mv4}Int molecules for the binding to the core-type sites. In addition, asymmetric binding of ^{mv4}Int, with the integrase able to form three complexes (I, II and III) with the right arm (B′/C′, FIG. 8D) thought at much lower efficiency than for the WT sites, but not with the left arms (B/C, FIG. 8EF). This behaviour is typical of YR systems and strongly suggests that one ^{mv4}Int monomer binds preferentially to the B′ or C′ core-binding site and promote the binding of the second ^{mv4}Int monomer at the B or C sites.

attPₘᵥ₄ contains two pairs of direct repeats of ^{mv4}Int Arm-Binding Sites.

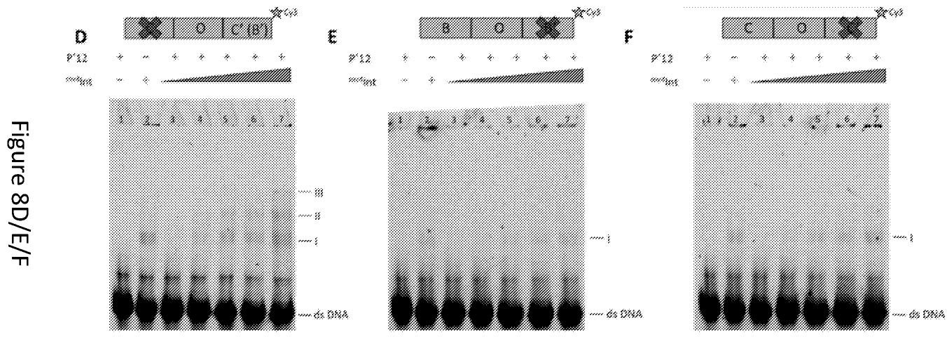

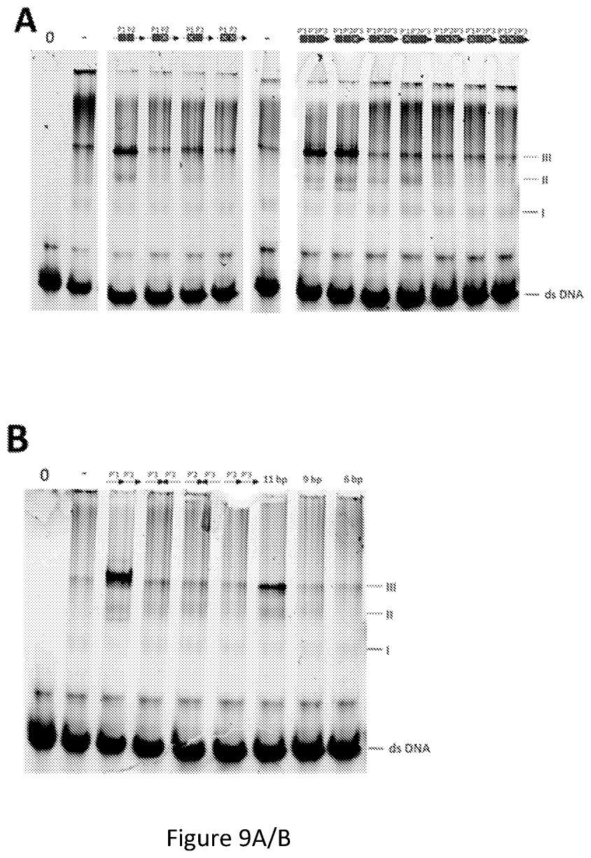

As the EMSA experiments allowed the Inventors to indirectly observe ^{mv4}Int binding to the arm-binding sites by stabilizing the binding of ^{mv4}Int to the core-type sites, they reanalysed the number and locations of the five arm-binding sites previously described (Auvray et al., 1999a), by studying eleven sets of ^{mv4}Int arm-binding sites (Table 7C). Among all the combinations used, only those containing either the P1-P2 pair or P′1-P′2 pair allow the stabilization of ^{mv4}Int binding to COC′ (FIG. 9A). This suggests that the P′3 sequence is not a P arm-binding site, in contrast to previous results (Auvray et al., 1999a). To characterize the binding of the N-ter domain on the P arm-type sites, different orientation of the P′1-P′2 pair was tested (i.e. direct repeats and inverted repeats), and results indicate that ^{mv4}Int binds cooperatively to the arm-binding sites only if P′1-P′2 sites are in direct repeats (FIG. 9B). The Inventors also used EMSA to characterize the size of the arm-binding sequence, and found that two direct repeats of 11 bp were necessary for the binding of ^{mv4}Int (FIG. 9B), suggesting that ^{mv4}Int binding sequence is a 11-bp word, in contrast to the 9-bp size previously proposed (Auvray et al., 1999a). In conclusion, the characterization of the DNA binding properties of ^{mv4}Int led us to show that attPₘᵥ₄ site has a more classical organization (FIG. 9C) than previously suggested (Auvray et al., 1999a; Coddeville et al., 2014a), with two pairs of adjacent arm-binding sites, one (P1P2) on the left arm (the P-arm) and the other (P′1P′2) on the right arm (the P′-arm), with a core region consisting of two ^{mv4}Int core-binding sites surrounding the 7-bp overlap sequence.

The ^{mv4}Int/attP system can be reprogrammed to target tRNA^{SER} of Other Bacterial Species

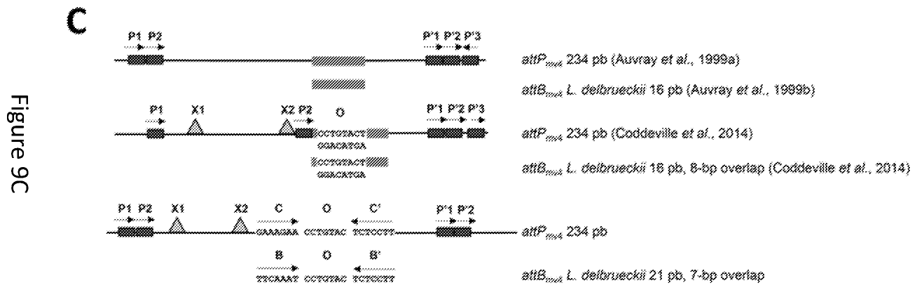

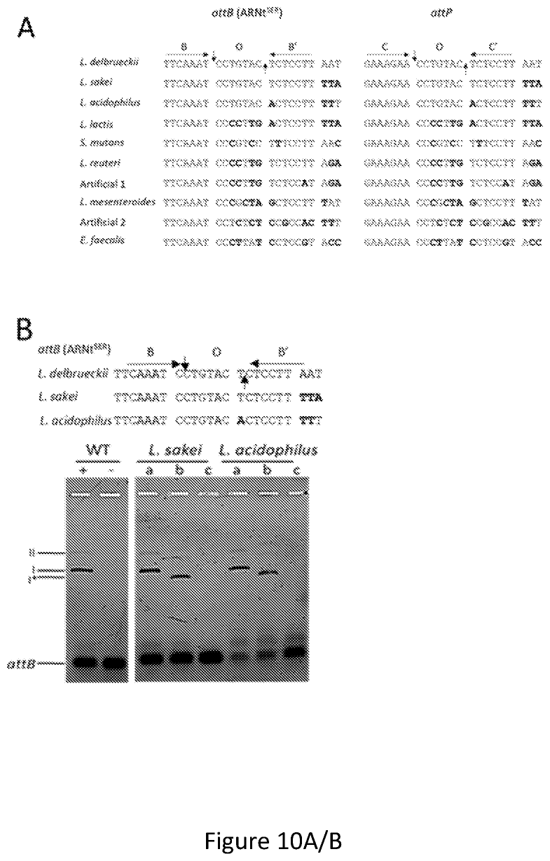

The characterization of the high degeneracy of mv4 attB site and core-attP region led us to postulate that ^{mv4}Int may be able to recombine DNA targets other than its cognate site by reprogramming the core-attP region, as long as these targets belong to the consensus pattern defined in this study (FIG. 6E). To test this hypothesis, the Inventors attempted to redirect the specificity of the in vitro recombination towards different bacterial tRNA^{SER} sites, from the most to the less conserved genes (FIG. 10A, left). To do this, they used different variants of attP with the overlap and C′ sequences modified to make them identical to the different overlap and B′ sequences (adapted attP, FIG. 10A, right) of the tRNA^{SER} tested. Seven native tRNA^{SER}sequences and two artificial sequences have been tested by in vitro recombination. Three different outputs have been obtained. First, some sites were permissive to site-specific recombination against either the native attPₘᵥ₄ site or its adapted counterpart (FIG. 10B), as the tRNA^{SER} from L. sakei and L. acidophilus. Such results were expected since these tRNAs are either identical to the native attB site (L. sakei) or contain the tolerated A at the first position of the B′ site (L. acidophilus). Second, several sites were permissive to recombination only against the adapted mv4 attP sites (lanes b, FIG. 10C), such as tRNA^{SER} from L. lactis, Streptococcus mutans, Lactobacillus reuteri, the artificial sequence 1, and Leuconostoc mesenteroides, though the latter site displays lower recombination efficiency. These attB sites contains up to four nucleotide differences on their overlap region compared to the native attB site, explaining why no recombination signal has been observed when tested against the native mv4 attP site (lanes a, FIG. 10C). Each of these sites contains one nucleotide modification into their B′ sequences, but all belong to the consensus pattern tolerated by ^{mv4}Int. The last output corresponds to attB sequences, one artificial and the tRNA^{SER} from E. faecalis, that were refractory to ^{mv4}Int-mediated recombination, even when using the adapted attP site (FIG. 10D). As both sequences contain two nucleotides out of the consensus pattern, it is tempting to postulate that ^{mv4}Int cannot tolerated more than one nucleotide that derogate the nucleotide constraint found in the degenerated pattern. Altogether, in vitro recombination results demonstrated that it is possible to reprogram the ^{mv4}Int-mediated site-specific recombination towards new tRNA^{SER} target sequences by adapting the 21-pb core region of the attPₘᵥ₄ site.

Reprogramming the Core-attP Site Allows In Vivo Recombination at the tRNA^{SER} of E. coli and L. lactis

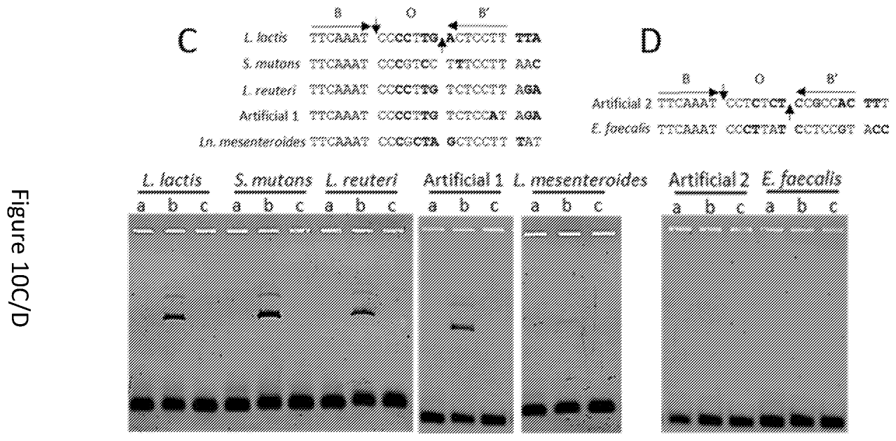

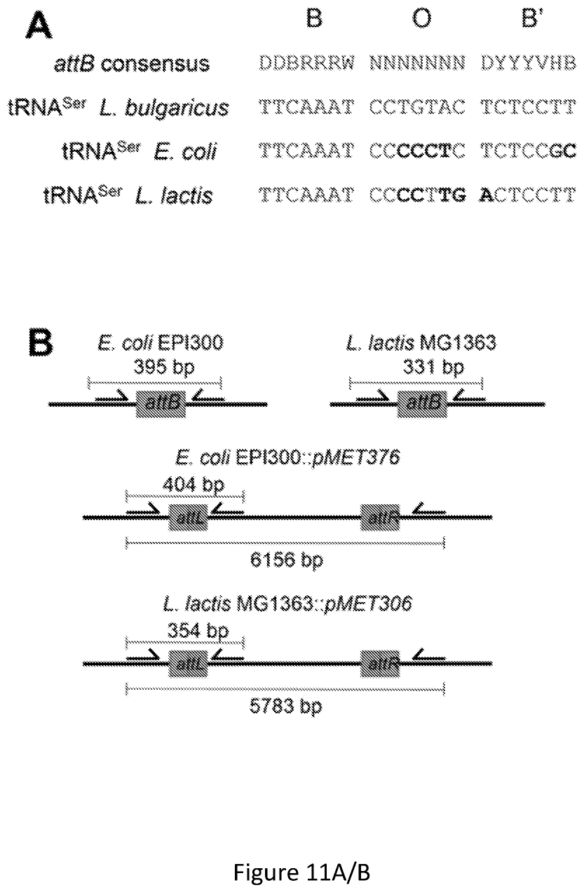

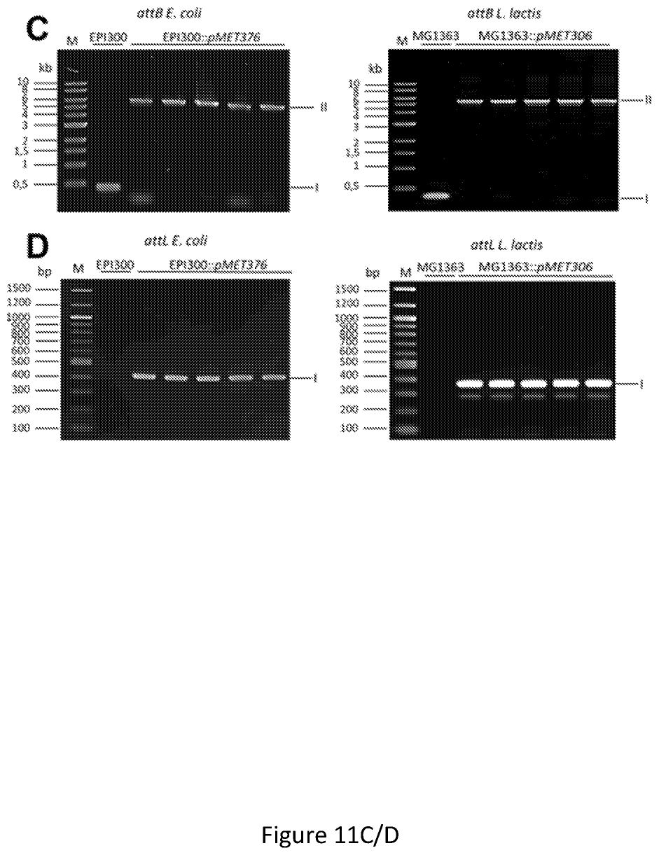

As ^{mv4}Int promote site-specific recombination in vitro into the L. lactis tRNA^{SER} when using the adapted attP site, the Inventors tested if this retargeted recombination could be performed in vivo. In addition, as the tRNA^{SER} of E. coli contains only one nucleotide that derogates the consensus pattern (FIG. 11A), but which is located at a less constrained position (6′ position, FIG. 6B), they hypothesised that reprogramming the ^{mv4}Int/attP system could lead to in vivo integration into the E. coli tRNA^{SER}. To do that, the overlap and C′ sequences of mv4 core-attP region of pMET359 plasmid (Table 6), a plasmid only able to replicate into the NEB5α-repA E. coli strain (Table 5), were replaced by the corresponding overlap and C′ sequences adapted to E. coli tRNA^{SER} (FIG. 11A), generating pMET376 (Table 6). For integration into the L. lactis genome, the overlap and C′ sequences adapted to L. lactis tRNA^{SER} (FIG. 11A) were used to replace the WT region of pMC1 plasmid to create pMET306 (Table 6). After transformation of these two suicide plasmids into their corresponding bacterial species, and selection of integrants based on antibiotic resistance, several clones were randomly picked and their genome structure at the tRNA^{SER} locus was analysed by PCR (FIG. 11BCD). In every case, all clones have integrated the plasmid at the expected target site, suggesting that tRNA^{SER} locus can be used as a “landing pad” to integrate foreign DNA by ^{mv4}Int/attP site-specific recombination in these two phylogenetically unrelated bacterial species.

Extending the ^{mv4}Int/attP system reprogramming to sequences other than tRNA^{SER}

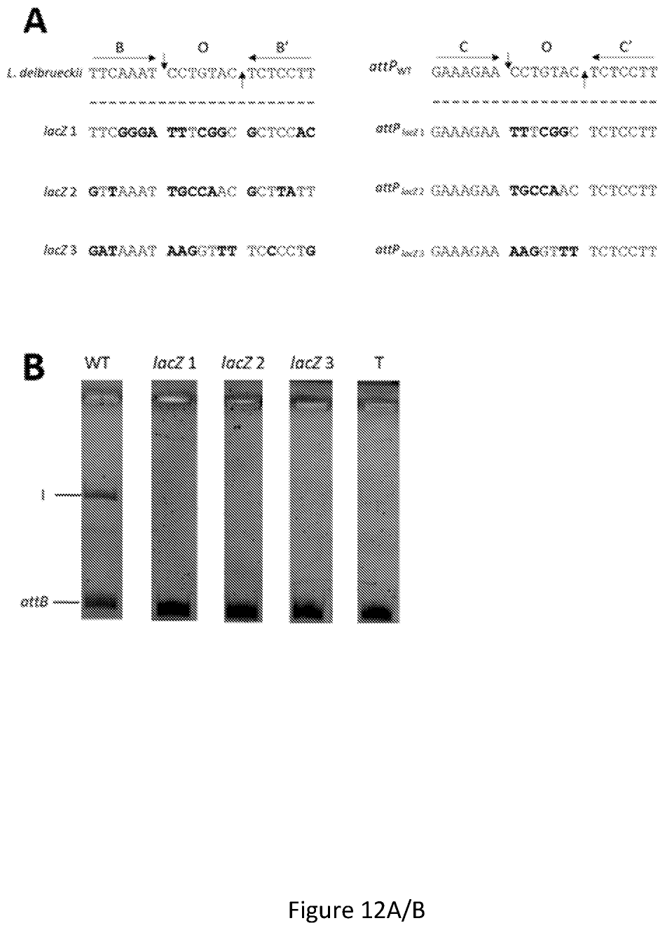

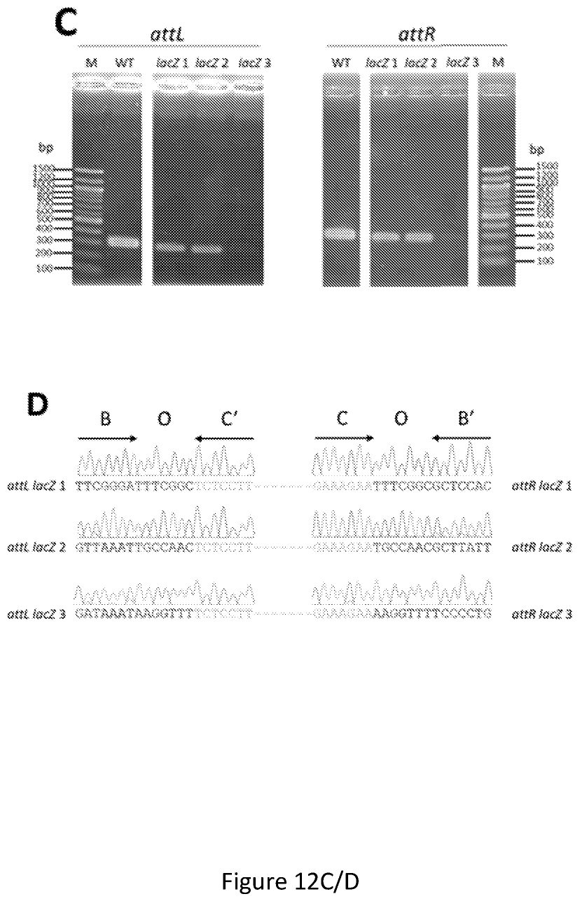

The Inventors performed an in-silico analysis of the E. coli MG1655 genome in order to identify putative recombination sites that obey the consensus pattern of attBₘᵥ₄. After analysis, 7959 putative sites have been identified, with three of them located into the lacZ gene. To test if the reaction can be reprogrammed to target these three sites in vitro, the overlap region of the mv4 core-attP was replaced by the overlap sequence of either lacZ1, lacZ2 and lacZ3 (FIG. 12A). One should note that, in contrast to the experiments performed with the tRNAs, the mv4 natural core-binding sites C and C′ were conserved. No recombined product could be observed for any attB/attP pair with our in vitro recombination assay (FIG. 12B), indicating either that no recombination occurred or that recombination efficiency was below the detection threshold (estimated as corresponding to less than 5% of recombination compared to the WT attB/attP pair, data not shown). However, recombined product could be detected for each target after PCR-amplification of the hybrid sites attL and attR, albeit with a very low intensity for the lacZ3 attB_{lacZ3}/attP_{lacZ}3 pair (FIG. 12C). The latter result was expected because they already have observed that replacement of the attBₘᵥ₄ site overlap by the sequence 5′-AAGGTTT-3′ induced a 15-fold decrease in recombination activity (FIG. 5E). Nevertheless, the sequence of each PCR-amplified hybrid site (FIG. 12D) confirmed that all the three lacZ putative sites were permissive to ^{mv4}Int-mediated site-specific recombination. These results show that it is possible to specifically reprogram the recombination reaction toward target sites meeting the requirements of the degenerated consensus sequence and exhibiting more than 50% of nucleotide difference with the attBₘᵥ₄ site, even if the recombination efficiency remains very low and only detectable by PCR-amplification. It seems reasonable to consider that such low recombination efficiency could be explained by the poor ranking of B and B′ target sites in the enrichment experiment (FIG. 7A) since, for example, although they belong to the reduced degeneracy motifs, B and B′ motif of lacZ2 site were ranked only 165^{th} and 477^{th} respectively, (FIG. 7B).

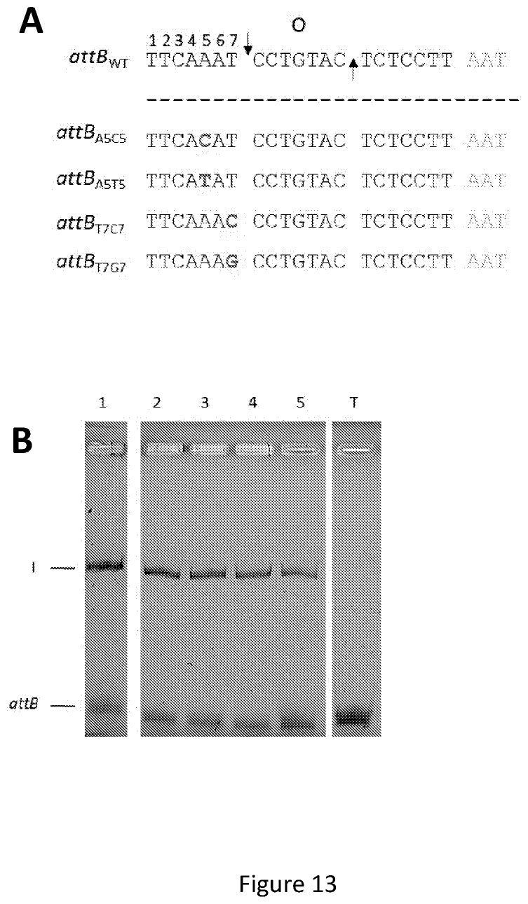

At last, the Inventors determined if targeted sequences containing one nucleotide that did not belong to the consensus patterns would still be productive in recombination, as two nucleotides excluded from the patterns seemed detrimental (see above). For that purpose, two nucleotide positions at the B site were independently replaced by “forbidden” nucleotides (FIG. 13A) and tested for in vitro recombination (FIG. 13B). In all cases, recombined product was observed at a significance level, indicating that one nucleotide out of the consensus pattern is not sufficient to impair recombination, probably through unpredictable compensatory effects.

TABLE 5
Strains.

| Strain | Relevant characteristics | Reference |
|---|---|---|
| E. coli | B F⁻ ompT gal dcm lon hsdSB(r_{B}−m_{B}−) λ(DE3 [lacI | (Studier et al., |
| BL21(DE3) | lacUV5-T7p07 ind1 sam7 nin5]) [malB⁺]_{K-12}(λ^{S}) | 1990) |
| E. coli | E. coli EC101, repA(pWV01) glgB::repA(pWV01), Kan^{R} | (Leenhouts et al., |
| EC1000 | | 1996) |
| E. coli | F− endA1 glnV44 thi-1 recA1 relA1 gyrA96 deoR | New England |
| Neb5α | nupG purB20 φ80dlacZΔM15 Δ(lacZYA- | Biolabs |
| | argF)U169, hsdR17(r_{K}−m_{K}+), λ− | |
| E. coli | F− λ− mcrA Δ(mrr-hsdRMS-mcrBC) | Epicentre |
| EPI300 | Φ80dlacZΔM15 Δ(lac)X74 recA1 endA1 | |
| | araD139 Δ(ara, leu)7697 galU galK rpsL (Str^{R}) | |
| | nupG trfA dhfr | |
| E. coli | Strain NEB5α glgB::repA(pWV01), Kan^{R} | This study |
| NEB5α-RepA | | |
| L. lactis | Wild Type strain. Plasmid-free derivative | (Gasson, 1983) |
| MG1363 | strain of NCDO712 | |

TABLE 6
Plasmids

| Name | Relevant properties | Reference |
|---|---|---|
| pBSattB | pBS::attBₘᵥ₄; Amp^{R} | (Dupont et al., 1995) |
| pMC1 | pRC1::^{mv4}Int-attPₘᵥ₄; Erm^{R} | (Dupont et al., 1995) |
| pET-Int | pET15b::^{mv4}Int; Amp^{R} | (Auvray et al., 1999a) |
| pCC1Fos | ori F-factor and oriV; Cm^{R} | Epicentre |
| pMET302 | pMC1::core attP tRNA^{SER} L. acidophilus; Erm^{R} | This study |
| pMET303 | pMC1::core attP artificial 2; Erm^{R} | This study |
| pMET304 | pMC1::core attP tRNA^{SER} L. mesenteroides; Erm^{R} | This study |
| pMET305 | pMC1::core attP tRNA^{SER} E. faecalis; Erm^{R} | This study |
| pMET306 | pMC1::core attP tRNA^{SER} L. lactis; Erm^{R} | This study |
| pMET307 | pMC1::core attP tRNA^{SER} S. mutans; Erm^{R} | This study |
| pMET308 | pMC1::core attP tRNA^{SER} L. reuteri; Erm^{R} | This study |
| pMET309 | pMC1::core attP artificial 1; Erm^{R} | This study |
| pMET310 | pMC1::core attP L. sakei; Erm^{R} | This study |
| pMET311 | pMC1 IS10::^{mv4}Int; Erm^{R} | This study |
| pMET320 | pBSattB::tRNA^{SER} L. acidophilus; Amp^{R} | This study |
| pMET321 | pBSattB::artificial 2; Amp^{R} | This study |
| pMET322 | pBSattB::tRNA^{SER} E. faecalis; Amp^{R} | This study |
| pMET323 | pBSattB::tRNA^{SER} L. lactis; Amp^{R} | This study |
| pMET324 | pBSattB::tRNA^{SER} S. mutans; Amp^{R} | This study |
| pMET325 | pBSattB::tRNA^{SER} L. mesenteroides; Amp^{R} | This study |
| pMET326 | pBSattB::tRNA^{SER} L. reuteri; Amp^{R} | This study |
| pMET327 | pBSattB::tRNA^{SER} L. sakei; Amp^{R} | This study |
| pMET328 | pBSattB::artificial 1; Amp^{R} | This study |
| pMET340 | pMC1 colE1::ori pWV01; Erm^{R} | This study |
| pMET348 | pCC1Fos::attBₘᵥ₄; Cm^{R} | This study |
| pMET349 | pCC1Fos::attBₘᵥ₄ 23 bp; Cm^{R} | This study |
| pMET357 | pCC1Fos::attBₘᵥ₄ 16 bp; Cm^{R} | This study |
| pMET359 | pMET340 + bla; Erm^{R}; Amp^{R} | This study |
| pMET364 | pMET348::overlap lacZ₁; Cm^{R} | This study |
| pMET365 | pMET348::overlap lacZ₃; Cm^{R} | This study |
| pMET366 | pMET348::attB lacZ₁; Cm^{R} | This study |
| pMET367 | pMET348::attB lacZ₂; Cm^{R} | This study |
| pMET368 | pMET348::attB lacZ₃; Cm^{R} | This study |
| pMET370 | pMET348::overlap lacZ₂; Cm^{R} | This study |
| pMET371 | pMET359::overlap lacZ₁; Erm^{R}, Amp^{R} | This study |
| pMET372 | pMET359::overlap lacZ₂; Erm^{R}, Amp^{R} | This study |
| pMET373 | pMET359::overlap lacZ₃; Erm^{R}, Amp^{R} | This study |
| pMET376 | pMET359::core attP tRNA^{SER} E. coli; Erm^{R}, Amp^{R} | This study |

TABLE 7
List of oligonucleotides
A. Oligonucleotides used for cloning.

| Primer name | Sequence (5′-3′) |
|---|---|
| SDM-attB4-F | GACCTGTACTCTCCTTAATAAGGTCAAATG (SEQ ID No 7) |
| SDM-attB4-R | GACCTTATTAAGGAGAGTACAGGTCTTGAAC (SEQ ID No 8) |
| SDM-attB5-F | CCTGTACTCTCCTGAATAAGGTCAAATGGTATC (SEQ ID No 9) |
| SDM-attB5-R | GACCTTATTAAGGAGAGTACAGGTCTTGAAC (SEQ ID No 10) |
| SDM-LbsakattB-F | CCTGTACTCTCCTTTTAAAGGTCAAATGGTATC (SEQ ID No 11) |
| SDM-LbsakattB-R | CATTTGACCTTTAAAAGGAGAGTACAGGATTTG (SEQ ID |
| | No 12) |
| SDM-LbacidoattB-F | CCTGTACACTCCTTTTTAAGGTCAAATGGTATC (SEQ ID No 13) |
| SDM-LbacidoattB-R | CATTTGACCTTAAAAAGGAGTGTACAGGATTTG (SEQ ID |
| | No 14) |
| SDM-LbreutattB-F | CCCCTTGTCTCCTTAGAAAGGTCAAATGGTATC (SEQ ID No 15) |
| SDM-LbreutattB-R | CTTTCTAAGGAGACAAGGGGATTTGAACCTGCG (SEQ ID |
| | No 16) |
| SDM-LlactisattB-F | CCCCTTGACTCCTTTTAAAGGTCAAATGGTATCC (SEQ ID |
| | No 17) |
| SDM-LlactisattB-R | CTTTAAAAGGAGTCAAGGGGATTTGAACCTGCG (SEQ ID |
| | No 18) |
| SDM-SmutansattB-F | CCCGTCCTTTCCTTAACAAGGTCAAATGGTATC (SEQ ID No 19) |
| SDM-SmutansattB-R | CTTGTTAAGGAAAGGACGGGATTTGAACCTGCG (SEQ ID |
| | No 20) |
| SDM-EfaecattB-F | CCCTTATCCTCCGTACCAAGGTCAAATGGTATCC (SEQ ID |
| | No 21) |
| SDM-EfaecattB-R | CTTGGTACGGAGGATAAGGGATTTGAACCTGCG (SEQ ID |
| | No 22) |
| SDM-LnmesentattB-F | CCCGCTAGCTCCTTTATAAGGTCAAATGGTATC (SEQ ID No 23) |
| SDM-LnmesentattB-R | CTTATAAAGGAGCTAGCGGGATTTGAACCTGCG (SEQ ID |
| | No 24) |
| SDM-EcoliattB-F | CCTCTCTCCGCCACTTTAAGGTCAAATGGTATCC (SEQ ID |
| | No 25) |
| SDM-EcoliattB-R | CTTAAAGTGGCGGAGAGAGGATTTGAACCTGCG (SEQ ID |
| | No 26) |
| SDMgibsonermAM-F | GAGAATATCGTCAACTGTTTACTAAAAATC (SEQ ID No 27) |
| SDMgibsonermAM-R | GTAAACAGTTGACGATATTCTCGATTG (SEQ ID No 28) |
| SDMgibsonattP5-F | AAAGAACCTGTACTCTCCTGAATCAAAGCAATAATC (SEQ ID |
| | No 29) |
| SDMgibsonattP5-R | TTCAGGAGAGTACAGGTTCTTTCAACCATGTTTC (SEQ ID |
| | No 30) |
| SDMgibsonattPsakei- | AAAGAACCTGTACTCTCCTTTTACAAAGCAATAATC (SEQ ID |
| F | No 31) |
| SDMgibsonattPsakei- | AAAAGGAGAGTACAGGTTCTTTCAACCATGTTTC (SEQ ID |
| R | No 32) |
| SDMgibsonattPacido- | AAAGAACCTGTACACTCCTTTTTCAAAGCAATAATC (SEQ ID |
| F | No 33) |
| SDMgibsonattPacido- | AAAAGGAGTGTACAGGTTCTTTCAACCATGTTTC (SEQ ID |
| R | No 34) |
| SDMgibsonattPmutans- | AAAGAACCCGTCCTTTCCTTAACCAAAGCAATAATCCC (SEQ |
| F | ID No 35) |
| SDMgibsonattPmutans- | TTAAGGAAAGGACGGGTTCTTTCAACCATGTTTC (SEQ ID |
| R | No 36) |
| SDMgibsonattPreuteri- | AAAGAACCCCTTGTCTCCTTAGACAAAGCAATAATCCC (SEQ |
| F | ID No 37) |
| SDMgibsonattPreuteri- | CTAAGGAGACAAGGGGTTCTTTCAACCATGTTTC (SEQ ID |
| R | No 38) |
| SDMgibsonattPlactis- | AAAGAACCCCTTGACTCCTTTTACAAAGCAATAATCCC (SEQ |
| F | ID No 39) |
| SDMgibsonattPlactis- | AAAAGGAGTCAAGGGGTTCTTTCAACCATGTTTC (SEQ ID |
| R | No 40) |
| SDMgibsonattPfaeca- | AAAGAACCCTTATCCTCCGTACCCAAAGCAATAATCCC (SEQ |
| F | ID No 41) |
| SDMgibsonattPfaeca- | GTACGGAGGATAAGGGTTCTTTCAACCATGTTTC (SEQ ID |
| R | No 42) |
| SDMgibsonattPcoli-F | AAAGAACCTCTCTCCGCCACTTTCAAAGCAATAATCCC (SEQ |
| | ID No 43) |
| SDMgibsonattPcoli-R | AAGTGGCGGAGAGAGGTTCTTTCAACCATGTTTC (SEQ ID |
| | No 44) |
| SDMgibsonattPmesent- | AAAGAACCCGCTAGCTCCTTTATCAAAGCAATAATCCC (SEQ |
| F | ID No 45) |
| SDMgibsonattPmesent- | TAAAGGAGCTAGCGGGTTCTTTCAACCATGTTTC (SEQ ID |
| R | No 46) |
| pBS-antiattB-F | GCTTGGGCTGCAGGA (SEQ ID No 47) |
| pBS-antiattB-R | GGAAAGGACATCTAAATCAAATGG (SEQ ID No 48) |
| SDM-Lbreuteri2attB-F | TCCATAGAAAGGTCAAATGGTATC (SEQ ID No 49) |
| SDM-Lbreuteri2attB-R | GACAAGGGGATTTGAACCTG (SEQ ID No 50) |
| oripGh9-F | TTAAATTTATACTGCAATCGGATGC (SEQ ID No 51) |
| oripGh9-R | GTATTTTTAATAGCCATGATATAATTACCTTATC (SEQ ID |
| | No 52) |
| pMC1-sans-colE1-F | TGGAACGAAAACTCACGTTAAG (SEQ ID No 53) |
| pMC1-sans-colE1-R | TGATTCTGTGGATAACCGTATTAC (SEQ ID No 54) |
| pCC1FOS-F | GTGGGATCCCCGGGTAC (SEQ ID No 55) |
| pCC1FOS-R | GTGGGATCCTCTAGAGTCGAC (SEQ ID No 56) |
| attB-gibsonpCC1Fos- | GCAGGTCGACTCTAGAGGATCCCACGAATTCCTGCAGCCCAA |
| F | GC (SEQ ID No 57) |
| attB-gibsonpCC1Fos- | GAGCTCGGTACCCGGGGATCCCACCCCCATTTGATTTAGATG |
| R | TCCTTTC (SEQ ID No 58) |
| attBWT-F | ATCCTGTACTCTCCTTAAT (SEQ ID No 59) |
| attBWT-R | ATTAAGGAGAGTACAGGAT (SEQ ID No 60) |
| attB-WTGGTT-F | GGTTCAAATCCTGTACTCTCCTTAAT (SEQ ID No 61) |
| attB-WTGGTT-R | ATTAAGGAGAGTACAGGATTTGAACC (SEQ ID No 62) |
| pMC1delTOPO-F | AAATCGAAACAGCAAAGAATGG (SEQ ID No 63) |
| Bla-F | TGGCACTTTTCGGGGAAATG (SEQ ID No 64) |
| Bla-R | GTGCTACAGAGTTCTTGAAGTG (SEQ ID No 65) |
| Site1LacZ-Gib-F | AAAGAATTTCGGCTCTCCTTAATCAAAGCAATAATCCC (SEQ |
| | ID No 66) |
| Site2LacZ-Gib-F | AAAGAATGCCAACTCTCCTTAATCAAAGCAATAATCCC (SEQ |
| | ID No 67) |
| Site3LacZ-Gib-F | AAAGAAAAGGTTTTCTCCTTAATCAAAGCAATAATCCC (SEQ |
| | ID No 68) |
| Site1LacZ-Gib-R | TTAAGGAGAGCCGAAATTCTTTCAACCATGTTTCTGGAG |
| | (SEQ ID No 69) |
| Site2LacZ-Gib-R | TTAAGGAGAGTTGGCATTCTTTCAACCATGTTTCTGGAG (SEQ |
| | ID No 70) |
| Site3LacZ-Gib-R | TTAAGGAGAAAACCTTTTCTTTCAACCATGTTTCTGGAG (SEQ |
| | ID No 71) |
| AmpGib-F | GAATTATGCAGTGCTGCCATAAC (SEQ ID No 72) |
| AmpGib-R | GTTATGGCAGCACTGCATAATTC (SEQ ID No 73) |
| OverlapSite1lacZ-R | AAGGAGAGCCGAAAATTTGAACCTGCGCACC (SEQ ID No 74) |
| OverlapSite2lacZ-R | AAGGAGAGTTGGCAATTTGAACCTGCGCACC (SEQ ID No 75) |
| OverlapSite3lacZ-R | AAGGAGAAAACCTTATTTGAACCTGCGCACC (SEQ ID No 76) |
| OverlapSite1lacZ-F | TCAAATTTTCGGCTCTCCTTAATAAGGTCAAATGGTATC (SEQ |
| | ID No 77) |
| OverlapSite2LacZ-F | TCAAATTGCCAACTCTCCTTAATAAGGTCAAATGGTATC (SEQ |
| | ID No 78) |
| OverlapSite3lacZ-F | TCAAATAAGGTTTTCTCCTTAATAAGGTCAAATGGTATC (SEQ |
| | ID No 79) |
| attBsite1LacZ-F | GAAATCCCGAACCTGCGCACCAATTCAACATTG (SEQ ID |
| | No 80) |
| attBsite1LacZ-R | GGCGCTCCACAATAAGGTCAAATGGTATCCCTATAGG (SEQ |
| | ID No 81) |
| attBsite2LacZ-F | GGCAATTTAACCCTGCGCACCAATTCAACATTG (SEQ ID No 82) |
| attBsite2LacZ-R | AACGCTTATTAATAAGGTCAAATGGTATCCCTATAGG (SEQ |
| | ID No 83) |
| attBsite3LacZ-F | CCTTATTTATCCCTGCGCACCAATTCAACATTG (SEQ ID No 84) |
| attBsite3LacZ-R | TTTTCCCCTGAATAAGGTCAAATGGTATCCCTATAGG (SEQ ID |
| | No 85) |

B. Oligonucleotides used for Randomised
DNA libraries construction and sequencing.

| Primer name | Sequence (5′-3′) |
|---|---|
| SeqbanqueattB-F | TGCCTGCAGGTCGACTCTAG (SEQ ID No 86) |
| SeqbanqueattL-R | ACGCTAATGCCATCTATTAACTAGC (SEQ ID No 87) |
| SeqbanqueattR-F | GAAACAACCAGAAACGCTTTTTAG (SEQ ID No 88) |
| SeqbanqueattB-R | GGCGAATTCGAGCTCGGTAC (SEQ ID No 89) |
| SeqbanqueattP-R | GGTCGACGGTATCGATAAGC (SEQ ID No 90) |
| SeqbanqueattP-F | GCAGGCGGAATGTTGAAAGAG (SEQ ID No 91) |
| attP-N10-N19-R | GTTCTGGAGGTTTCGAATCTTG (SEQ ID No 92) |
| attP-N10-N19-Vf-F | GTGATAATCGCCTGCCCGTTTGAC (SEQ ID No 93) |
| attPlibrary-F | CAAGATTCGAAACCTCCAGAAC (SEQ ID No 94) |
| attPlibrary-R | GTCAAACGGGCAGGCGATTATCAC (SEQ ID No 95) |
| attBlibrary-F | GCAGGTCGACTCTAGAGGATCCCACAAGCTTCGAAATCCGCCGA |
| | ACCAATG (SEQ ID No 96) |
| attBlibrary-R | GAGCTCGGTACCCGGGGATCCCACGAATTCACTACTGGCTACTT |
| | TGAAATACTTCC (SEQ ID No 97) |
| attB-gibsonpCC1Fos-F | GCAGGTCGACTCTAGAGGATCCCACGAATTCCTGCAGCCCAAGC |
| | (SEQ ID No 57) |
| attB-gibsonpCC1Fos-R | GAGCTCGGTACCCGGGGATCCCACCCCCATTTGATTTAGATGTC |
| | CTTTC (SEQ ID No 58) |
| attB Lib9 | CGAAATCCGCCGAACCAATGTTGANNNNNNNNGCAGGTTCANN |
| | NNNTGTACTCTCCNNNNNAAGGTCAAATNNNNNNNNTATAGGA |
| | AGTATTTCAAAGTAGCCAGTAGT (SEQ ID No 98) |
| attB Lib7 | CGAAATCCGCCGAACCAATGTTGANNNNNNNCGCAGGTTCAAA |
| | TNNNNNNNTCTCCTTAATAAGGTCAAATGNNNNNNNTATAGGA |
| | AGTATTTCAAAGTAGCCAGTAGT (SEQ ID No 99) |
| attB Lib8 | CGAAATCCGCCGAACCAATGTTGANNNNNNNCGCAGGTTCAAA |
| | TCCTGTACNNNNNNNAATAAGGTCAAATGNNNNNNNTATAGGA |
| | AGTATTTCAAAGTAGCCAGTAGT (SEQ ID No 100) |
| attP Lib2 | CAAGATTCGAAACCTCCAGAACCCTCCAGAAACATGGTTGAAAG |
| | AANNNNNNNTCTCCTTAATCAAAGCAATAATCCCCGAGAAATCA |
| | ACATTCTCGGGGATTATTTTTGTTTTTAACTAGAAAATAACTAGA |
| | AAGAGCTAGTTAATAGATGGCATTAGCGTGATAATCGCCTGCCC |
| | GTTTGAC (SEQ ID No 101) |
| attP Lib1 | CAAGATTCGAAACCTCCAGAACCCTCCAGAAACATGGTTGAAAG |
| | AACCTGTACNNNNNNNAATCAAAGCAATAATCCCCGAGAAATC |
| | AACATTCTCGGGGATTATTTTTGTTTTTAACTAGAAAATAACTAG |
| | AAAGAGCTAGTTAATAGATGGCATTAGCGTGATAATCGCCTGCC |
| | CGTTTGAC (SEQ ID No 102) |
| attB Lib5 | CGAAATCCGCCGAACCAATGTTGAATTGGTGNNNNNNNNNNAA |
| | TCCTGTACTCTCCTTAATAAGGTCAAATGGTATCCCTATAGGAAG |
| | TATTTCAAAGTAGCCAGTAGT (SEQ ID No 103) |
| attP Lib4 | CAAGATTCGAAACCTCCAGAACCCTCCAGAAACNNNNNNNNNN |
| | GAACCTGTACTCTCCTTAATCAAAGCAATAATCCCCGAGAAATC |
| | AACATTCTCGGGGATTATTTTTGTTTTTAACTAGAAAATAACTAG |
| | AAAGAGCTAGTTAATAGATGGCATTAGCGTGATAATCGCCTGCC |
| | CGTTTGAC (SEQ ID No 104) |
| attB Lib6 | CGAAATCCGCCGAACCAATGTTGAATTGGTGCGCNNNNNNNNN |
| | NCCTGTACTCTCCTTAATAAGGTCAAATGGTATCCCTATAGGAA |
| | GTATTTCAAAGTAGCCAGTAGT (SEQ ID No 105) |
| attP Lib3 | CAAGATTCGAAACCTCCAGAACCCTCCAGAAACATGGTTNNNNN |
| | NNCCTGTACTCTCCTTAATCAAAGCAATAATCCCCGAGAAATCA |
| | ACATTCTCGGGGATTATTTTTGTTTTTAACTAGAAAATAACTAGA |
| | AAGAGCTAGTTAATAGATGGCATTAGCGTGATAATCGCCTGCCC |
| | GTTTGAC (SEQ ID No 106) |

C. Oligonucleotides used EMSA.

| Primer name | Sequence (5′-3′) |
|---|---|
| COC′WT-F | GGTATTGGAAAGAACCTGTACTCTCCTTGCGTAAC (SEQ ID |
| | No 107) |
| COC′WT-Cy3-R | GTTACGCAAGGAGAGTACAGGTTCTTTCCAATACC (SEQ ID |
| | No 108) |
| P′12WT-28 bp-F | GTTTTTAACTAGAAAATAACTAGAATTC (SEQ ID No 109) |
| P′12WT-28 bp-R | GAATTCTAGTTATTTTCTAGTTAAAAAC (SEQ ID No 110) |
| P′12WT-40 bp-F | CACGTCGTTTTTAACTAGAAAATAACTAGAATTCCACGTC |
| | (SEQ ID No 111) |
| P′12WT-40 bp-R | GACGTGGAATTCTAGTTATTTTCTAGTTAAAAACGACGTG |
| | (SEQ ID No 112) |
| P′12WTdelP′3-F | TTTTTAACTAGAAAATAACTAGAAAGCGACCGAGCGGTCG |
| | (SEQ ID No 113) |
| P′12WTdelP′3-R | CGACCGCTCGGTCGCTTTCTAGTTATTTTCTAGTTAAAAA |
| | (SEQ ID No 114) |
| P′123WT-F | TTTTTAACTAGAAAATAACTAGAAAGAGCTAGTTAATAGA |
| | (SEQ ID No 115) |
| P′123WT-R | TCTATTAACTAGCTCTTTCTAGTTATTTTCTAGTTAAAAA |
| | (SEQ ID No 116) |
| P′1WT-F | TTTTTAACTAGAACGACCGAGCGGAGGCGGCACGCTGTCG |
| | (SEQ ID No 117) |
| P′1WT-R | CGACAGCGTGCCGCCTCCGCTCGGTCGTTCTAGTTAAAAA |
| | (SEQ ID No 118) |
| P′2WT-F | GCCGACCGAGCGGAATAACTAGAAAGGCGGCACGCTGTCG |
| | (SEQ ID No 119) |
| P′2WT-R | CGACAGCGTGCCGCCTTTCTAGTTATTCCGCTCGGTCGGC |
| | (SEQ ID No 120) |
| P′3WT-F | GCCGACCGAGCGGGCGGCACGCTGAGAGCTAGTTAATAGA |
| | (SEQ ID No 121) |
| P′3WT-R | TCTATTAACTAGCTCTCAGCGTGCCGCCCGCTCGGTCGGC |
| | (SEQ ID No 122) |
| P′23WT-F | GCCGACCGAGCGGAATAACTAGAAAGAGCTAGTTAATAGA |
| | (SEQ ID No 123) |
| P′23WT-R | TCTATTAACTAGCTCTTTCTAGTTATTCCGCTCGGTCGGC |
| | (SEQ ID No 124) |
| P′13WT-F | TTTTTAACTAGAACGACCGAGCGGAGAGCTAGTTAATAGA |
| | (SEQ ID No 125) |
| P′13WT-R | TCTATTAACTAGCTCTCCGCTCGGTCGTTCTAGTTAAAAA |
| | (SEQ ID No 126) |
| P12WT-F | CACGTCGTGATCAACTAGATTTTTAACTAGAAACCACGTC |
| | (SEQ ID No 127) |
| P12WT-R | GACGTGGTTTCTAGTTAAAAATCTAGTTGATCACGACGTG |
| | (SEQ ID No 128) |
| P1WT-F | CACGTCGTGATCAACTAGATTCGACCGAGCGGACCACGTC |
| | (SEQ ID No 129) |
| P1WT-R | GACGTGGTCCGCTCGGTCGAATCTAGTTGATCACGACGTG |
| | (SEQ ID No 130) |
| P2WT-F | CACGTCGTGCGACCGAGCGGTTTTAACTAGAAACCACGTC |
| | (SEQ ID No 131) |
| P2WT-R | GACGTGGTTTCTAGTTAAAACCGCTCGGTCGCACGACGTG |
| | (SEQ ID No 132) |
| Nositebras-F | CACGTCGTGCGACCGAGCGGTGCGGCACGCTGACCACGTC |
| | (SEQ ID No 133) |
| Nositebras-R | GACGTGGTCAGCGTGCCGCACCGCTCGGTCGCACGACGTG |
| | (SEQ ID No 134) |
| P′12WT-40 bp-R- | CACGTCGTTTTTAACTAGAAAATAACTAGAATTCCACGTC |
| Cy3 | (SEQ ID No 135) |
| COC′WT-R | GTTACGCAAGGAGAGTACAGGTTCTTTCCAATACC (SEQ ID |
| | No 136) |
| BOB′WT-F | GGTATTGTTCAAATCCTGTACTCTCCTTGCGTAAC (SEQ ID |
| | No 137) |
| BOB′WT-R | GTTACGCAAGGAGAGTACAGGATTTGAACAATACC (SEQ |
| | ID No 138) |
| BOB′WT-R-Cy3 | GTTACGCAAGGAGAGTACAGGATTTGAACAATACC (SEQ |
| | ID No 139) |
| XOC′-F | GGTATTGGAACCCGCCTGTACTCTCCTTGCGTAAC (SEQ ID |
| | No 140) |
| XOC′-R-Cy3 | GTTACGCAAGGAGAGTACAGGCGGGTTCCAATACC (SEQ ID |
| | No 141) |
| COX-F | GGTATTGGAAAGAACCTGTACCGGGCTTGCGTAAC (SEQ ID |
| | No 142) |
| COX-R-Cy3 | GTTACGCAAGCCCGGTACAGGTTCTTTCCAATACC (SEQ ID |
| | No 143) |
| BOX-F | GGTATTGTTCAAATCCTGTACCGGGCTTGCGTAAC (SEQ ID |
| | No 144) |
| BOX-R-Cy3 | GTTACGCAAGCCCGGTACAGGATTTGAACAATACC (SEQ ID |
| | No 145) |
| P12-9 pb-F | CACGTCGTGCTCAACTAGAGTCTTAACTAGAGACCACGTC |
| | (SEQ ID No 146) |
| P12-9 pb-R | GACGTGGTCTCTAGTTAAGACTCTAGTTGAGCACGACGTG |
| | (SEQ ID No 147) |
| P12-6 pb-F | CACGTCGTGCCGAACTAGGGTCCGAACTAGGGACCACGTC |
| | (SEQ ID No 148) |
| P12-6 pb-R | GACGTGGTCCCTAGTTCGGACCCTAGTTCGGCACGACGTG |
| | (SEQ ID No 149) |
| P′23forward-F | GCCGACCGAGCGGAATAACTAGAAAGATTAACTAGCTAGA |
| | (SEQ ID No 150) |
| P′23forward-R | TCTAGCTAGTTAATCTTTCTAGTTATTCCGCTCGGTCGGC |
| | (SEQ ID No 151) |
| P′12reverse-F | TTTTTAACTAGAATTCTAGTTATTAGCGACCGAGCGGTCG |
| | (SEQ ID No 152) |
| P′12reverse-R | CGACCGCTCGGTCGCTAATAACTAGAATTCTAGTTAAAAA |
| | (SEQ ID No 153) |

D. Oligonucleotides used for
in vitro fluorescent recombination.

| Primer name | Sequence |
|---|---|
| LbbulgattB-F | GAATTCCTGCAGCCCAAGC (SEQ ID No 154) |
| Cy3-New-attB-R | GATGTAGATAATTTTTGGGCCAAGG (SEQ ID No 155) |

E. Oligonucleotides used to validate
in vivo integration into tRNASER

| Primer name | Sequence (5′-3′) |
|---|---|
| ARNtSERcoli-F | ACAGTGACGATCTAACCCTTC (SEQ ID No 156) |
| ARNtSERcoli-R | TGACTAATTTGCTTTGTTCCTG (SEQ ID No 157) |
| ARNtSERlactis-F | CATCATTTTTCTTCTTTCAAATTAATATAAATGC (SEQ ID |
| | No 158) |
| ARNtSERlactis-R | CAGGAGGAAAAGGAGTAAGC (SEQ ID No 159) |
| attL-R | ACGCTAATGCCATCTATTAACTAGC (SEQ ID No 160) |

Bold nucleotides indicate the localization of arm or core-binding sites as well as the overlap sequence.
Underlined nucleotides are nucleotides that differ from the WT sites.

TABLE 8
NGS data of the randomized DNA libraries.

| DNA | | Motif number | | | | | | Motif number | |
|---|---|---|---|---|---|---|---|---|---|
| Library | Motif number | after | | Motif occurrence | | | | representing 50% | |
| (core- | before | recombination | | after recombination | | | | of the read count | |
| binding | recombination | (attL or attR) | | Rep1 | | Rep2 | | after recombination | |
| region) | (attB or attP) | Rep1 | Rep2 | min | max | min | max | Rep1 | Rep2 |
| attB | 16384 | 5459 | 6955 | 1 | 14405 | 1 | 11737 | 196 | 199 |
| Lib6 | (100%) | (33.32%) | (42.45%) | | | | | (3.59%) | (2.86%) |
| (B) | | | | | | | | | |
| attB | 16312 | 9497 | 10085 | 1 | 49310 | 1 | 69525 | 371 | 460 |
| Lib8 | (99.56%) | (57.97%) | (61.55%) | | | | | (3.91%) | (4.56%) |
| (B′) | | | | | | | | | |
| attP | 16340 | 10416 | 11781 | 1 | 2139 | 1 | 3925 | 784 | 799 |
| Lib3 | (99.73%) | (63.57%) | (71.91%) | | | | | (7.53%) | (6.78%) |
| (C) | | | | | | | | | |
| attP | 16315 | 7549 | 7247 | 1 | 16157 | 1 | 46672 | 254 | 167 |
| Lib1 | (99.58%) | (46.08%) | (44.23%) | | | | | (3.36%) | (2.30%) |
| (C′) | | | | | | | | | |

### REFERENCES

Afgan, E., Baker, D., Batut, B., van den Beek, M., Bouvier, D., Cech, M., et al. (2018) The Galaxy platform for accessible, reproducible and collaborative biomedical analyses: 2018 update. Nucleic Acids Research 46: W537-W544 https://doi.org/10.1093/nar/gky379. Accessed Feb. 2, 2022.
Aihara, H., Kwon, H. J., Nunes-Diby, S. E., Landy, A., and Ellenberger, T. (2003) A conformational switch controls the DNA cleavage activity of lambda integrase. Mol Cell 12: 187-198.
Auvray, F., Coddeville, M., Espagno, G., and Ritzenthaler, P. (1999a) Integrative recombination of Lactobacillus delbrueckii bacteriophage mv4: functional analysis of the reaction and structure of the attP site. Mol Gen Genet 262: 355-366.
Auvray, F., Coddeville, M., Ordonez, R. C., and Ritzenthaler, P. (1999b) Unusual structure of the attB site of the site-specific recombination system of Lactobacillus delbrueckii bacteriophage mv4. J Bacteriol 181: 7385-7389.
Auvray, F., Coddeville, M., Ritzenthaler, P., and Dupont, L. (1997) Plasmid integration in a wide range of bacteria mediated by the integrase of Lactobacillus delbrueckii bacteriophage mv4. J Bacteriol 179: 1837-1845.
Bauer, C. E., Gardner, J. F., and Gumport, R. I. (1985) Extent of sequence homology required for bacteriophage lambda site-specific recombination. J Mol Biol 181: 187-197.
Biswas, T., Aihara, H., Radman-Livaja, M., Filman, D., Landy, A., and Ellenberger, T. (2005) A structural basis for allosteric control of DNA recombination by k integrase. Nature 435: 1059-1066 https://www.nature.com/articles/nature03657. Accessed Aug. 10, 2021.
Cluzel, P.-J., Veaux, M., Rousseau, M., and Accolas, J.-P. (1987) Evidence for temperate bacteriophages in two strains of Lactobacillus bulgaricus. J Dairy Res 54: 397-405.
Coddeville, M., and Ritzenthaler, P. (2010) Control of directionality in bacteriophage mv4 site-specific recombination: functional analysis of the Xis factor. J Bacteriol 192: 624-635.
Coddeville, M., Spinella, J.-F., Cassart, P., Girault, G., Daveran-Mingot, M.-L., Le Bourgeois, P., and Ritzenthaler, P. (2014a) Bacteriophage mv4 site-specific recombination: the central role of the P2 mv4Int-binding site. J Virol 88: 1839-1842.
Coddeville, M., Spinella, J. F., Cassart, P., Girault, G., Daveran-Mingot, M. L., Le Bourgeois, P., and Ritzenthaler, P. (2014b) Bacteriophage mv4 site-specific recombination: the central role of the P2 ^{mv4}Int-binding site. J Virol 88: 1839-1842.
Craig, N. L., and Nash, H. A. (1983) The mechanism of phage lambda site-specific recombination: site-specific breakage of DNA by Int topoisomerase. Cell 35: 795-803.
Crooks, G. E., Hon, G., Chandonia, J.-M., and Brenner, S. E. (2004) WebLogo: a sequence logo generator. Genome Res 14: 1188-1190.
Datsenko, K. A., and Wanner, B. L. (2000) One-step inactivation of chromosomal genes in Escherichia coli K-12 using PCR products. Proc Natl Acad Sci USA 97: 6640-6645.
Dupont, L., Boizet-Bonhoure, B., Coddeville, M., Auvray, F., and Ritzenthaler, P. (1995a) Characterization of genetic elements required for site-specific integration of Lactobacillus delbrueckii subsp. bulgaricus bacteriophage mv4 and construction of an integration-proficient vector for Lactobacillus plantarum. J Bacteriol 177: 586-595.
Dupont, L., Boizet-Bonhoure, B., Coddeville, M., Auvray, F., and Ritzenthaler, P. (1995b) Characterization of genetic elements required for site-specific integration of Lactobacillus delbrueckii subsp. bulgaricus bacteriophage mv4 and construction of an integration-proficient vector for Lactobacillus plantarum. J Bacteriol 177: 586-595.
Gasson, M. J. (1983) Plasmid complements of Streptococcus lactis NCDO 712 and other lactic streptococci after protoplast-induced curing. J Bacteriol 154: 1-9.
Gibb, B., Gupta, K., Ghosh, K., Sharp, R., Chen, J., and Van Duyne, G. D. (2010) Requirements for catalysis in the Cre recombinase active site. Nucleic Acids Res 38: 5817-5832.
Gibson, D. G., Young, L., Chuang, R.-Y., Venter, J. C., Hutchison, C. A., and Smith, H. O. (2009) Enzymatic assembly of DNA molecules up to several hundred kilobases. Nat Methods 6: 343-345.
Grindley, N. D. F., Whiteson, K. L., and Rice, P. A. (2006) Mechanisms of site-specific recombination. Annu Rev Biochem 75: 567-605.
Hauser, M. A., and Scocca, J. J. (1992) Site-specific integration of the Haemophilus influenzae bacteriophage HP1. Identification of the points of recombinational strand exchange and the limits of the host attachment site. J Biol Chem 267: 6859-6864.
Hoess, R. H., Wierzbicki, A., and Abremski, K. (1986) The role of the loxP spacer region in P1 site-specific recombination. Nucleic Acids Res 14: 2287-2300.
Jumper, J., Evans, R., Pritzel, A., Green, T., Figurnov, M., Ronneberger, O., et al. (2021a) Highly accurate protein structure prediction with AlphaFold. Nature 596: 583-589 http://www.nature.com/articles/s41586-021-03819-2. Accessed Jan. 16, 2022.
Jumper, J., Evans, R., Pritzel, A., Green, T., Figurnov, M., Ronneberger, O., et al. (2021b) Highly accurate protein structure prediction with AlphaFold. Nature 596: 583-589.
Kolot, M., Malchin, N., Elias, A., Gritsenko, N., and Yagil, E. (2015) Site promiscuity of coliphage HK022 integrase as a tool for gene therapy. Gene Ther 22: 521-527.
Kolot, M., and Yagil, E. (1994) Position and direction of strand exchange in bacteriophage HK022 integration. Mol Gen Genet 245: 623-627.
Le Bourgeois, P., Langella, P., and Ritzenthaler, P. (2000) Electrotransformation of Lactococcus lactis. In Electrotransformation of Bacteria. Eynard, N., and Teissié, J. (eds). Springer, Berlin, Heidelberg. pp. 56-65 https://doi.org/10.1007/978-3-662-04305-9_6. Accessed Sep. 7, 2021.
Lee, G., and Saito, I. (1998) Role of nucleotide sequences of loxP spacer region in Cre-mediated recombination. Gene 216: 55-65.
Leenhouts, K., Buist, G., Bolhuis, A., Berge, A. ten, Kiel, J., Mierau, I., et al. (1996) A general system for generating unlabelled gene replacements in bacterial chromosomes. Mol Gen Genet 253: 217-224 https://doi.org/10.1007/s004380050315. Accessed Sep. 7, 2021.
Malanowska, K., Salyers, A. A., and Gardner, J. F. (2006) Characterization of a conjugative transposon integrase, IntDOT. Mol Microbiol 60: 1228-1240.
Mata, M., Trautwetter, A., Luthaud, G., and Ritzenthaler, P. (1986) Thirteen virulent and temperate bacteriophages of Lactobacillus bulgaricus and Lactobacillus lactis belong to a single DNA homology group. Appl Environ Microbiol 52: 812-818.
McLeod, M., Craft, S., and Broach, J. R. (1986) Identification of the crossover site during FLP-mediated recombination in the Saccharomyces cerevisiae plasmid 2 microns circle. Mol Cell Biol 6: 3357-3367.
Nunes-Duby, S. E., Kwon, H. J., Tirumalai, R. S., Ellenberger, T., and Landy, A. (1998) Similarities and differences among 105 members of the Int family of site-specific recombinases. Nucleic Acids Res 26: 391-406.
Nunes-Duby, S. E., Kwon, H. J., Tirumalai, R. S., Ellenberger, T., and Landy, A. (1998) Similarities and differences among 105 members of the Int family of site-specific recombinases. Nucleic Acids Res 26: 391-406.
Peña, C. E., Stoner, J. E., and Hatfull, G. F. (1996) Positions of strand exchange in mycobacteriophage L5 integration and characterization of the attB site. J Bacteriol 178: 5533-5536.
Rice, P., Longden, I., and Bleasby, A. (2000) EMBOSS: The European Molecular Biology Open Software Suite. Trends in Genetics 16: 276-277 http://www.cell.com/trends/genetics/abstract/S0168-9525(00)02024-2. Accessed Feb. 2, 2022.
Sarkar, D., Radman-Livaja, M., and Landy, A. (2001) The small DNA binding domain of lambda integrase is a context-sensitive modulator of recombinase functions. EMBO J 20: 1203-1212.
Schneider, T. D., and Stephens, R. M. (1990) Sequence logos: a new way to display consensus sequences. Nucleic Acids Res 18: 6097-6100.
Sheren, J., Langer, S. J., and Leinwand, L. A. (2007) A randomized library approach to identifying functional lox site domains for the Cre recombinase. Nucleic Acids Res 35: 5464-5473.
Smith-Mungo, L., Chan, I. T., and Landy, A. (1994) Structure of the P22 att site. Conservation and divergence in the lambda motif of recombinogenic complexes. J Biol Chem 269: 20798-20805.
Studier, F. W., Rosenberg, A. H., Dunn, J. J., and Dubendorff, J. W. (1990) Use of T7 RNA polymerase to direct expression of cloned genes. In Methods in Enzymology. Academic Press, pp. 60-89 https://www.sciencedirect.com/science/article/pii/007668799085008C. Accessed May 30, 2022.
Weisberg, R. A., Enquist, L. W., Foeller, C., and Landy, A. (1983) Role for DNA homology in site-specific recombination. The isolation and characterization of a site affinity mutant of coliphage lambda. J Mol Biol 170: 319-342.
Wojciak, J. M., Sarkar, D., Landy, A., and Clubb, R. T. (2002) Arm-site binding by lambda-integrase: solution structure and functional characterization of its amino-terminal domain. Proc Natl Acad Sci USA 99: 3434-3439.
Zhu, B., Cai, G., Hall, E. O., and Freeman, G. J. (2007) In-Fusion™ assembly: seamless engineering of multidomain fusion proteins, modular vectors, and mutations. BioTechniques 43: 354-359 https://www.future-science.com/doi/10.2144/000112536. Accessed Jun. 3, 2022.

### Sequences

SEQ ID No 1: nucleotide sequence of
tRNASER(CGA) of the Lactobacillus delbrueckii
subsp. bulgaricus
GGAGAGTTGGCAGAGCGGTAATGCAGCGGACTCGAAATCCGCCGA
ACCAATGTTGAATTGGTGCGCAGGTTCAAATCCTGTACTCTCCTT
AAT
SEQ ID No 2: polynucleotide fragment P1-P2
5′-ATCAACTAGATTTTTAACTAGAA-3′
SEQ ID No 3: polynucleotide fragment P′1-P′2
5′-TTTAACTAGAAAATAACTAGAA-3′
SEQ ID No 4: nucleotide sequence of ^{mv4}Int
ATGCCAAAGCGTAATCCTGCAATCAAAAAATACACCAGCCGGGGC
CAAACAAAATACAAATTCCAGATTTACCTGGGCCAGGACGAAAGC
GGAAAATCAATCAACACGACCCGGAGTGGTTTTAAATCTTACTCC
CAAGCATCAGCAGCTTACAACAAGCTTAAGGCCCAAGGATTGGCC
GCCAAAGCACCCAAAAAAGCGACCACCGATGAGGTGTGGTCGCTT
TGGTTTGATAGCTATAAAGGCGGAGTTAAAGAGTCGTCAGCAAAC
AAAACGCTGACTAGTTACAGAGTCCACATCAAGCCTGCTTTTGGT
GATAAAATGATCAGCTCGATCAAGACGGCCACCGTACAACTCTGG
GCAAACAATTTGGCCACCAAGCTGGTCAACTACAAGGTGGTTGTG
CGCCTGCTAGGGACTCTTTTTGAATTTGCCAAGCGCCTGGACTAT
TGCAAGGACAACCCGGTCAAGCAGATCATCATGCCAAAAGCTACC
TCCAGGCCTCGCAGAGACATCAGCACCAACTACTATAACCGTGAT
GAGCTTCAGCAGTTCCTGCAGGCCGCTAAAGAAGTAGGATCCCGG
ACTTATGTCTTCTTTCTACTCCTTGCTACCACGGGCCTCCGAAAA
GGCGAAGCACTAGCCCTGGATTGGTCGGACATCGACTACGATCAA
GGAAAAATCTCCGTCACTAAGACTCTTGCCTATGGCCTGGGTGGC
AAGTACGGGATCCAGCCACCTAAGACTAAGGCAGGGATCCGCACG
GTGCCACTGACTGATCAGATGGCAGCCGTTTTAAAAGACTACCAT
AGTGATCTCTGCCCGCACCTTTTTCACACGCTTGATGGTGATTAT
CTCCGTCTTAGTAAGCCAGATCAGTGGCTTCAGGCTGTTTATAAA
CACGACCCAGACCTCCGACAAATTAGAATCCATGGCTTCCGTCAT
ACTTTTGCGTCCCTGCTCATCACTGCGGATCCGTCAATCAAGCCA
ACAGACGTGCAAGCAATCCTGGGTCATGAATCAATCGATATTACC
ATGGAGATTTACATGCACGCCACTCAAGAAGGCAGGCGGAATGTT
GAAAGAGTTCTAAATCAACTAGATTTTTAA
SEQ ID No 5: peptide sequence of ^{mv4}Int
MPKRNPAIKKYTSRGQTKYKFQIYLGQDESGKSINTTRSGFKSYS
QASAAYNKLKAQGLAAKAPKKATTDEVWSLWFDSYKGGVKESSAN
KTLTSYRVHIKPAFGDKMISSIKTATVQLWANNLATKLVNYKVVV
RLLGTLFEFAKRLDYCKDNPVKQIIMPKATSRPRRDISTNYYNRD
ELQQFLQAAKEVGSRTYVFFLLLATTGLRKGEALALDWSDIDYDQ
GKISVTKTLAYGLGGKYGIQPPKTKAGIRTVPLTDQMAAVLKDYH
SDLCPHLFHTLDGDYLRLSKPDQWLQAVYKHDPDLRQIRIHGFRH
TFASLLITADPSIKPTDVQAILGHESIDITMEIYMHATQEGRRNV
ERVLNQLDF

1. A method for preparing a site-specific recombination polynucleotide molecule comprising the steps of:
a—selecting a DNA target site in a genome of a bacterial host cell having a sequence of B—O—B′ wherein:
B is 5′-X1-X1-X2-X3-X3-X3-X4-3′ wherein at most 1 of the nucleic acids of B may be N;
O is 5′-N—N—N—N—N—N—N-3′ and
B′ is 5′-X1-X5-X5-X5-X6-X7-X2-3′ wherein at most 1 of the nucleic acids of B′ may be N;
wherein X1 to X7 and N have independently the following definitions:
X1 is A or G or T;
X2 is C or G or T;
X3 is A or G;
X4 is A or T;
X5 is C or T;
X6 is A or C or G;
X7 is A or C or T; and
N is A or C or G or T;
b—providing the site-specific recombination polynucleotide molecule having a sequence of C—O—C′ wherein:
C is 5′-X1-X1-X2-X1-X3-X1-X4-3′ wherein at most 1 of the nucleic acids of C may be N;
O is 5′-N—N—N—N—N—N—N-3′; and
C′ is 5′-X1-X5-X5-X5-X6-X7-X5-3′ wherein at most 1 of the nucleic acids of C′ may be N;
and wherein X1, X2, X3, X4, X5, X6, X7 and N are as defined previously;
and wherein O of C—O—C′ is identical to O of B—O—B′ of the bacterial host cell.

2. The method for preparing a site-specific recombination polynucleotide molecule according to claim 1, wherein B′ is 5′-X4-X5-X5-X5-X6-X7-X2-3′ and wherein at most 1 of the nucleic acids of B′ may be N; C is 5′-X1-X1-X8-X1-X3-X1-X4-3′ wherein at most 1 of the nucleic acids of C may be N; and C′ is 5′-X4-X5-X5-X5-X9-X7-X5-3′ wherein at most 1 of the nucleic acids of C′ may be N; and wherein X8 is T or G and X9 is A or C.

3. A kit for site-specific recombination of at least one polynucleotide sequence of interest into a genome of a bacterial host cell comprising:
A—a polynucleotide molecule A comprising:
(i) a sequence of between 220 to 250 pb comprising polynucleotide fragments P1-P2, C—O—C′ and P′ 1-P′2 wherein:
P1-P2 is
(SEQ ID No 2)
5′-ATCAACTAGATTTTTAACTAGAA-3′;
C—O—C′ is the site-specific recombination polynucleotide molecule according to claim 1 or 2; and
P′1-P′2 is
(SEQ ID No 3)
5′-TTTAACTAGAAAATAACTAGAA-3′;
the sequence interacting with the DNA target site according to claim 1 or 2 in the bacterial host cell for integrating the polynucleotide sequence of interest; and
(ii) at least one polynucleotide sequence of interest;
B—a polynucleotide molecule int having at least 80%, preferably at least 85%, 90%, 95% or 100% identity with the sequence of SEQ ID No 4 coding for ^{mv4}Int or the ^{mv4}Int of SEQ ID No 5.

4. The kit of claim 3, wherein the polynucleotide molecule A is inserted in a first vector.

5. The kit of claim 4, wherein the polynucleotide molecule int coding for ^{mv4}Int is inserted in the first vector or in a second vector.

6. A method for integrating a polynucleotide sequence of interest into a genome of a genetically modified bacterial host cell comprising:
a—preparing a vector comprising a polynucleotide molecule A comprising:
(i) a sequence of between 220 to 250 pb comprising the following polynucleotide fragments P1-P2, C—O—C′ and P′1-P′2 wherein:
P1-P2 is
(SEQ ID No 2)
5′-ATCAACTAGATTTTTAACTAGAA-3′;
C—O—C′ is the site-specific recombination polynucleotide molecule according to claim 1 or 2; and
P′1-P′2 is
(SEQ ID No 3)
5′-TTTAACTAGAAAATAACTAGAA-3′;
(ii) at least one polynucleotide sequence of interest;
b—transforming the bacterial host cell with the vector obtained at step (a) and the polynucleotide molecule int of SEQ ID No 4 coding for ^{mv4}Int;
c—maintaining the transformed host cell under conditions that allow integration of the polynucleotide sequence of interest into the genome of the host cell.

7. A genetically modified bacterial host cell obtained by the method of claim 6,
wherein the genetically modified bacterial host cell comprises a vector comprising a polynucleotide molecule A comprising:
(i) a sequence of between 220 to 250 pb comprising the following polynucleotide fragments P1-P2, C—O—C′ and P′1-P′2 wherein:
P1-P2 is
(SEQ ID No 2)
5′-ATCAACTAGATTTTTAACTAGAA-3′;
C—O—C′ is the site-specific recombination polynucleotide molecule according to claim 1 or 2; and
P′1-P′2 is
(SEQ ID No 3)
5′-TTTAACTAGAAAATAACTAGAA-3′;
(ii) at least one polynucleotide sequence of interest;
and the polynucleotide molecule INT of SEQ ID No 4 coding for ^{mv4}Int.


SEQUENCE LISTING (251 sequences)

SEQ ID NO: 1 - DNA, 93 bp, Lactobacillus delbrueckii
  source 1..93: mol_type = genomic DNA; sub_species = Lactobacillus delbrueckii subsp. bulgaricus
ggagagttggcagagcggtaatgcagcggactcgaaatccgccgaaccaatgttgaattggtgcgcaggttcaaatcctgtactctccttaat

SEQ ID NO: 2 - DNA, 23 bp, Lactobacillus delbrueckii
  source 1..23: mol_type = genomic DNA; sub_species = subsp. bulgaricus
atcaactagatttttaactagaa

SEQ ID NO: 3 - DNA, 22 bp, Lactobacillus delbrueckii
  source 1..22: mol_type = genomic DNA; sub_species = subsp. bulgaricus
tttaactagaaaataactagaa

SEQ ID NO: 4 - DNA, 1110 bp, Lactobacillus delbrueckii
  source 1..1110: mol_type = genomic DNA; sub_species = subsp. bulgaricus
atgccaaagcgtaatcctgcaatcaaaaaatacaccagccggggccaaacaaaatacaaattccagatttacctgggccaggacgaaagcggaaaatcaatcaacacgacccggagtggttttaaatcttactcccaagcatcagcagcttacaacaagcttaaggcccaaggattggccgccaaagcacccaaaaaagcgaccaccgatgaggtgtggtcgctttggtttgatagctataaaggcggagttaaagagtcgtcagcaaacaaaacgctgactagttacagagtccacatcaagcctgcttttggtgataaaatgatcagctcgatcaagacggccaccgtacaactctgggcaaacaatttggccaccaagctggtcaactacaaggtggttgtgcgcctgctagggactctttttgaatttgccaagcgcctggactattgcaaggacaacccggtcaagcagatcatcatgccaaaagctacctccaggcctcgcagagacatcagcaccaactactataaccgtgatgagcttcagcagttcctgcaggccgctaaagaagtaggatcccggacttatgtcttctttctactccttgctaccacgggcctccgaaaaggcgaagcactagccctggattggtcggacatcgactacgatcaaggaaaaatctccgtcactaagactcttgcctatggcctgggtggcaagtacgggatccagccacctaagactaaggcagggatccgcacggtgccactgactgatcagatggcagccgttttaaaagactaccatagtgatctctgcccgcacctttttcacacgcttgatggtgattatctccgtcttagtaagccagatcagtggcttcaggctgtttataaacacgacccagacctccgacaaattagaatccatggcttccgtcatacttttgcgtccctgctcatcactgcggatccgtcaatcaagccaacagacgtgcaagcaatcctgggtcatgaatcaatcgatattaccatggagatttacatgcacgccactcaagaaggcaggcggaatgttgaaagagttctaaatcaactagatttttaa

SEQ ID NO: 5 - protein, 369 aa, Lactobacillus delbrueckii
  source 1..369: note = subsp. bulgaricus
MPKRNPAIKKYTSRGQTKYKFQIYLGQDESGKSINTTRSGFKSYSQASAAYNKLKAQGLAAKAPKKATTDEVWSLWFDSYKGGVKESSANKTLTSYRVHIKPAFGDKMISSIKTATVQLWANNLATKLVNYKVVVRLLGTLFEFAKRLDYCKDNPVKQIIMPKATSRPRRDISTNYYNRDELQQFLQAAKEVGSRTYVFFLLLATTGLRKGEALALDWSDIDYDQGKISVTKTLAYGLGGKYGIQPPKTKAGIRTVPLTDQMAAVLKDYHSDLCPHLFHTLDGDYLRLSKPDQWLQAVYKHDPDLRQIRIHGFRHTFASLLITADPSIKPTDVQAILGHESIDITMEIYMHATQEGRRNVERVLNQLDF

SEQ ID NO: 6 - DNA, 21 bp, Lactobacillus delbrueckii
  source 1..21: mol_type = genomic DNA; sub_species = subsp. bulgaricus
ttcaaatcctgtactctcctt

SEQ ID NO: 7 - DNA, 30 bp, synthetic construct
  misc_feature 1..30: note = Oligonucleotides used for cloning
  source 1..30: mol_type = other DNA
gacctgtactctccttaataaggtcaaatg

SEQ ID NO: 8 - DNA, 31 bp, synthetic construct
  misc_feature 1..31: note = Oligonucleotides used for cloning
  source 1..31: mol_type = other DNA
gaccttattaaggagagtacaggtcttgaac

SEQ ID NO: 9 - DNA, 33 bp, synthetic construct
  misc_feature 1..33: note = Oligonucleotides used for cloning
  source 1..33: mol_type = other DNA
cctgtactctcctgaataaggtcaaatggtatc

SEQ ID NO: 10 - DNA, 31 bp, synthetic construct; identical to SEQ ID NO: 8
  misc_feature 1..31: note = Oligonucleotides used for cloning
  source 1..31: mol_type = other DNA

SEQ ID NO: 11 - DNA, 33 bp, synthetic construct
  misc_feature 1..33: note = Oligonucleotides used for cloning
  source 1..33: mol_type = other DNA
cctgtactctccttttaaaggtcaaatggtatc

SEQ ID NO: 12 - DNA, 33 bp, synthetic construct
  misc_feature 1..33: note = Oligonucleotides used for cloning
  source 1..33: mol_type = other DNA
catttgacctttaaaaggagagtacaggatttg

SEQ ID NO: 13 - DNA, 33 bp, synthetic construct
  misc_feature 1..33: note = Oligonucleotides used for cloning
  source 1..33: mol_type = other DNA
cctgtacactcctttttaaggtcaaatggtatc

SEQ ID NO: 14 - DNA, 33 bp, synthetic construct
  misc_feature 1..33: note = Oligonucleotides used for cloning
  source 1..33: mol_type = other DNA
catttgaccttaaaaaggagtgtacaggatttg

SEQ ID NO: 15 - DNA, 33 bp, synthetic construct
  misc_feature 1..33: note = Oligonucleotides used for cloning
  source 1..33: mol_type = other DNA
ccccttgtctccttagaaaggtcaaatggtatc

SEQ ID NO: 16 - DNA, 33 bp, synthetic construct
  misc_feature 1..33: note = Oligonucleotides used for cloning
  source 1..33: mol_type = other DNA
ctttctaaggagacaaggggatttgaacctgcg

SEQ ID NO: 17 - DNA, 34 bp, synthetic construct
  misc_feature 1..34: note = Oligonucleotides used for cloning
  source 1..34: mol_type = other DNA
ccccttgactccttttaaaggtcaaatggtatcc

SEQ ID NO: 18 - DNA, 33 bp, synthetic construct
  misc_feature 1..33: note = Oligonucleotides used for cloning
  source 1..33: mol_type = other DNA
ctttaaaaggagtcaaggggatttgaacctgcg

SEQ ID NO: 19 - DNA, 33 bp, synthetic construct
  misc_feature 1..33: note = Oligonucleotides used for cloning
  source 1..33: mol_type = other DNA
cccgtcctttccttaacaaggtcaaatggtatc

SEQ ID NO: 20 - DNA, 33 bp, synthetic construct
  misc_feature 1..33: note = Oligonucleotides used for cloning
  source 1..33: mol_type = other DNA
cttgttaaggaaaggacgggatttgaacctgcg

SEQ ID NO: 21 - DNA, 34 bp, synthetic construct
  misc_feature 1..34: note = Oligonucleotides used for cloning
  source 1..34: mol_type = other DNA
cccttatcctccgtaccaaggtcaaatggtatcc

SEQ ID NO: 22 - DNA, 33 bp, synthetic construct
  misc_feature 1..33: note = Oligonucleotides used for cloning
  source 1..33: mol_type = other DNA
cttaaagtggcggagagaggatttgaacctgcg

SEQ ID NO: 23 - DNA, 33 bp, synthetic construct
  misc_feature 1..33: note = Oligonucleotides used for cloning
  source 1..33: mol_type = other DNA
cccgctagctcctttataaggtcaaatggtatc

SEQ ID NO: 24 - DNA, 33 bp, synthetic construct
  misc_feature 1..33: note = Oligonucleotides used for cloning
  source 1..33: mol_type = other DNA
cttataaaggagctagcgggatttgaacctgcg

SEQ ID NO: 25 - DNA, 34 bp, synthetic construct
  misc_feature 1..34: note = Oligonucleotides used for cloning
  source 1..34: mol_type = other DNA
cctctctccgccactttaaggtcaaatggtatcc

SEQ ID NO: 26 - DNA, 33 bp, synthetic construct; identical to SEQ ID NO: 22
  misc_feature 1..33: note = Oligonucleotides used for cloning
  source 1..33: mol_type = other DNA

SEQ ID NO: 27 - DNA, 30 bp, synthetic construct
  misc_feature 1..30: note = Oligonucleotides used for cloning
  source 1..30: mol_type = other DNA
gagaatatcgtcaactgtttactaaaaatc

SEQ ID NO: 28 - DNA, 27 bp, synthetic construct
  misc_feature 1..27: note = Oligonucleotides used for cloning
  source 1..27: mol_type = other DNA
gtaaacagttgacgatattctcgattg

SEQ ID NO: 29 - DNA, 36 bp, synthetic construct
  misc_feature 1..36: note = Oligonucleotides used for cloning
  source 1..36: mol_type = other DNA
aaagaacctgtactctcctgaatcaaagcaataatc

SEQ ID NO: 30 - DNA, 34 bp, synthetic construct
  misc_feature 1..34: note = Oligonucleotides used for cloning
  source 1..34: mol_type = other DNA
ttcaggagagtacaggttctttcaaccatgtttc

SEQ ID NO: 31 - DNA, 36 bp, synthetic construct
  misc_feature 1..36: note = Oligonucleotides used for cloning
  source 1..36: mol_type = other DNA
aaagaacctgtactctccttttacaaagcaataatc

SEQ ID NO: 32 - DNA, 34 bp, synthetic construct
  misc_feature 1..34: note = Oligonucleotides used for cloning
  source 1..34: mol_type = other DNA
aaaaggagagtacaggttctttcaaccatgtttc

SEQ ID NO: 33 - DNA, 36 bp, synthetic construct
  misc_feature 1..36: note = Oligonucleotides used for cloning
  source 1..36: mol_type = other DNA
aaagaacctgtacactcctttttcaaagcaataatc

SEQ ID NO: 34 - DNA, 34 bp, synthetic construct
  misc_feature 1..34: note = Oligonucleotides used for cloning
  source 1..34: mol_type = other DNA
aaaaggagtgtacaggttctttcaaccatgtttc

SEQ ID NO: 35 - DNA, 38 bp, synthetic construct
  misc_feature 1..38: note = Oligonucleotides used for cloning
  source 1..38: mol_type = other DNA
aaagaacccgtcctttccttaaccaaagcaataatccc

SEQ ID NO: 36 - DNA, 34 bp, synthetic construct
  misc_feature 1..34: note = Oligonucleotides used for cloning
  source 1..34: mol_type = other DNA
ttaaggaaaggacgggttctttcaaccatgtttc

SEQ ID NO: 37 - DNA, 38 bp, synthetic construct
  misc_feature 1..38: note = Oligonucleotides used for cloning
  source 1..38: mol_type = other DNA
aaagaaccccttgtctccttagacaaagcaataatccc

SEQ ID NO: 38 - DNA, 34 bp, synthetic construct
  misc_feature 1..34: note = Oligonucleotides used for cloning
  source 1..34: mol_type = other DNA
ctaaggagacaaggggttctttcaaccatgtttc

SEQ ID NO: 39 - DNA, 38 bp, synthetic construct
  misc_feature 1..38: note = Oligonucleotides used for cloning
  source 1..38: mol_type = other DNA
aaagaaccccttgactccttttacaaagcaataatccc

SEQ ID NO: 40 - DNA, 34 bp, synthetic construct
  misc_feature 1..34: note = Oligonucleotides used for cloning
  source 1..34: mol_type = other DNA
aaaaggagtcaaggggttctttcaaccatgtttc

SEQ ID NO: 41 - DNA, 38 bp, synthetic construct
  misc_feature 1..38: note = Oligonucleotides used for cloning
  source 1..38: mol_type = other DNA
aaagaacccttatcctccgtacccaaagcaataatccc

SEQ ID NO: 42 - DNA, 34 bp, synthetic construct
  misc_feature 1..34: note = Oligonucleotides used for cloning
  source 1..34: mol_type = other DNA
gtacggaggataagggttctttcaaccatgtttc

SEQ ID NO: 43 - DNA, 38 bp, synthetic construct
  misc_feature 1..38: note = Oligonucleotides used for cloning
  source 1..38: mol_type = other DNA
aaagaacctctctccgccactttcaaagcaataatccc

SEQ ID NO: 44 - DNA, 34 bp, synthetic construct
  misc_feature 1..34: note = Oligonucleotides used for cloning
  source 1..34: mol_type = other DNA
aagtggcggagagaggttctttcaaccatgtttc

SEQ ID NO: 45 - DNA, 38 bp, synthetic construct
  misc_feature 1..38: note = Oligonucleotides used for cloning
  source 1..38: mol_type = other DNA
aaagaacccgctagctcctttatcaaagcaataatccc

SEQ ID NO: 46 - DNA, 34 bp, synthetic construct
  misc_feature 1..34: note = Oligonucleotides used for cloning
  source 1..34: mol_type = other DNA
taaaggagctagcgggttctttcaaccatgtttc

SEQ ID NO: 47 - DNA, 15 bp, synthetic construct
  misc_feature 1..15: note = Oligonucleotides used for cloning
  source 1..15: mol_type = other DNA
gcttgggctgcagga

SEQ ID NO: 48 - DNA, 24 bp, synthetic construct
  misc_feature 1..24: note = Oligonucleotides used for cloning
  source 1..24: mol_type = other DNA
ggaaaggacatctaaatcaaatgg

SEQ ID NO: 49 - DNA, 24 bp, synthetic construct
  misc_feature 1..24: note = Oligonucleotides used for cloning
  source 1..24: mol_type = other DNA
tccatagaaaggtcaaatggtatc

SEQ ID NO: 50 - DNA, 20 bp, synthetic construct
  misc_feature 1..20: note = Oligonucleotides used for cloning
  source 1..20: mol_type = other DNA
gacaaggggatttgaacctg

SEQ ID NO: 51 - DNA, 25 bp, synthetic construct
  misc_feature 1..25: note = Oligonucleotides used for cloning
  source 1..25: mol_type = other DNA
ttaaatttatactgcaatcggatgc

SEQ ID NO: 52 - DNA, 34 bp, synthetic construct
  misc_feature 1..34: note = Oligonucleotides used for cloning
  source 1..34: mol_type = other DNA
gtatttttaatagccatgatataattaccttatc

SEQ ID NO: 53 - DNA, 22 bp, synthetic construct
  misc_feature 1..22: note = Oligonucleotides used for cloning
  source 1..22: mol_type = other DNA
tggaacgaaaactcacgttaag

SEQ ID NO: 54 - DNA, 24 bp, synthetic construct
  misc_feature 1..24: note = Oligonucleotides used for cloning
  source 1..24: mol_type = other DNA
tgattctgtggataaccgtattac

SEQ ID NO: 55 - DNA, 17 bp, synthetic construct
  misc_feature 1..17: note = Oligonucleotides used for cloning
  source 1..17: mol_type = other DNA
gtgggatccccgggtac

SEQ ID NO: 56 - DNA, 21 bp, synthetic construct
  misc_feature 1..21: note = Oligonucleotides used for cloning
  source 1..21: mol_type = other DNA
gtgggatcctctagagtcgac

SEQ ID NO: 57 - DNA, 44 bp, synthetic construct
  misc_feature 1..44: note = Oligonucleotides used for cloning
  source 1..44: mol_type = other DNA
gcaggtcgactctagaggatcccacgaattcctgcagcccaagc

SEQ ID NO: 58 - DNA, 49 bp, synthetic construct
  misc_feature 1..49: note = Oligonucleotides used for cloning
  source 1..49: mol_type = other DNA
gagctcggtacccggggatcccacccccatttgatttagatgtcctttc

SEQ ID NO: 59 - DNA, 19 bp, synthetic construct
  misc_feature 1..19: note = Oligonucleotides used for cloning
  source 1..19: mol_type = other DNA
atcctgtactctccttaat

SEQ ID NO: 60 - DNA, 19 bp, synthetic construct
  misc_feature 1..19: note = Oligonucleotides used for cloning
  source 1..19: mol_type = other DNA
attaaggagagtacaggat

SEQ ID NO: 61 - DNA, 26 bp, synthetic construct
  misc_feature 1..26: note = Oligonucleotides used for cloning
  source 1..26: mol_type = other DNA
ggttcaaatcctgtactctccttaat

SEQ ID NO: 62 - DNA, 26 bp, synthetic construct
  misc_feature 1..26: note = Oligonucleotides used for cloning
  source 1..26: mol_type = other DNA
attaaggagagtacaggatttgaacc

SEQ ID NO: 63 - DNA, 22 bp, synthetic construct
  misc_feature 1..22: note = Oligonucleotides used for cloning
  source 1..22: mol_type = other DNA
aaatcgaaacagcaaagaatgg

SEQ ID NO: 64 - DNA, 20 bp, synthetic construct
  misc_feature 1..20: note = Oligonucleotides used for cloning
  source 1..20: mol_type = other DNA
tggcacttttcggggaaatg

SEQ ID NO: 65 - DNA, 22 bp, synthetic construct
  misc_feature 1..22: note = Oligonucleotides used for cloning
  source 1..22: mol_type = other DNA
gtgctacagagttcttgaagtg

SEQ ID NO: 66 - DNA, 38 bp, synthetic construct
  misc_feature 1..38: note = Oligonucleotides used for cloning
  source 1..38: mol_type = other DNA
aaagaatttcggctctccttaatcaaagcaataatccc

SEQ ID NO: 67 - DNA, 38 bp, synthetic construct
  misc_feature 1..38: note = Oligonucleotides used for cloning
  source 1..38: mol_type = other DNA
aaagaatgccaactctccttaatcaaagcaataatccc

SEQ ID NO: 68 - DNA, 38 bp, synthetic construct
  misc_feature 1..38: note = Oligonucleotides used for cloning
  source 1..38: mol_type = other DNA
aaagaaaaggttttctccttaatcaaagcaataatccc

SEQ ID NO: 69 - DNA, 39 bp, synthetic construct
  misc_feature 1..39: note = Oligonucleotides used for cloning
  source 1..39: mol_type = other DNA
ttaaggagagccgaaattctttcaaccatgtttctggag

SEQ ID NO: 70 - DNA, 39 bp, synthetic construct
  misc_feature 1..39: note = Oligonucleotides used for cloning
  source 1..39: mol_type = other DNA
ttaaggagagttggcattctttcaaccatgtttctggag

SEQ ID NO: 71 - DNA, 39 bp, synthetic construct
  misc_feature 1..39: note = Oligonucleotides used for cloning
  source 1..39: mol_type = other DNA
ttaaggagaaaaccttttctttcaaccatgtttctggag

SEQ ID NO: 72 - DNA, 23 bp, synthetic construct
  misc_feature 1..23: note = Oligonucleotides used for cloning
  source 1..23: mol_type = other DNA
gaattatgcagtgctgccataac

SEQ ID NO: 73 - DNA, 23 bp, synthetic construct
  misc_feature 1..23: note = Oligonucleotides used for cloning
  source 1..23: mol_type = other DNA
gttatggcagcactgcataattc

SEQ ID NO: 74 - DNA, 31 bp, synthetic construct
  misc_feature 1..31: note = Oligonucleotides used for cloning
  source 1..31: mol_type = other DNA
aaggagagccgaaaatttgaacctgcgcacc

SEQ ID NO: 75 - DNA, 31 bp, synthetic construct
  misc_feature 1..31: note = Oligonucleotides used for cloning
  source 1..31: mol_type = other DNA
aaggagagttggcaatttgaacctgcgcacc

SEQ ID NO: 76 - DNA, 31 bp, synthetic construct
  misc_feature 1..31: note = Oligonucleotides used for cloning
  source 1..31: mol_type = other DNA
aaggagaaaaccttatttgaacctgcgcacc

SEQ ID NO: 77 - DNA, 39 bp, synthetic construct
  misc_feature 1..39: note = TCAAATTTTCGGCTCTCCTTAATAAGGTCAAATGGTATC
  source 1..39: mol_type = other DNA
tcaaattttcggctctccttaataaggtcaaatggtatc

SEQ ID NO: 78 - DNA, 39 bp, synthetic construct
  misc_feature 1..39: note = Oligonucleotides used for cloning
  source 1..39: mol_type = other DNA
tcaaattgccaactctccttaataaggtcaaatggtatc

SEQ ID NO: 79 - DNA, 39 bp, synthetic construct
  misc_feature 1..39: note = Oligonucleotides used for cloning
  source 1..39: mol_type = other DNA
tcaaataaggttttctccttaataaggtcaaatggtatc

SEQ ID NO: 80 - DNA, 33 bp, synthetic construct
  misc_feature 1..33: note = Oligonucleotides used for cloning
  source 1..33: mol_type = other DNA
gaaatcccgaacctgcgcaccaattcaacattg

SEQ ID NO: 81 - DNA, 37 bp, synthetic construct
  misc_feature 1..37: note = Oligonucleotides used for cloning
  source 1..37: mol_type = other DNA
ggcgctccacaataaggtcaaatggtatccctatagg

SEQ ID NO: 82 - DNA, 33 bp, synthetic construct
  misc_feature 1..33: note = Oligonucleotides used for cloning
  source 1..33: mol_type = other DNA
ggcaatttaaccctgcgcaccaattcaacattg

SEQ ID NO: 83 - DNA, 37 bp, synthetic construct
  misc_feature 1..37: note = Oligonucleotides used for cloning
  source 1..37: mol_type = other DNA
aacgcttattaataaggtcaaatggtatccctatagg

SEQ ID NO: 84 - DNA, 33 bp, synthetic construct
  misc_feature 1..33: note = Oligonucleotides used for cloning
  source 1..33: mol_type = other DNA
ccttatttatccctgcgcaccaattcaacattg

SEQ ID NO: 85 - DNA, 37 bp, synthetic construct
  misc_feature 1..37: note = Oligonucleotides used for cloning
  source 1..37: mol_type = other DNA
ttttcccctgaataaggtcaaatggtatccctatagg

SEQ ID NO: 86 - DNA, 20 bp, synthetic construct
  misc_feature 1..20: note = Oligonucleotides used for Randomised DNA libraries constructionand sequencing
  source 1..20: mol_type = other DNA
tgcctgcaggtcgactctag

SEQ ID NO: 87 - DNA, 25 bp, synthetic construct
  misc_feature 1..25: note = Oligonucleotides used for Randomised DNA libraries constructionand sequencing
  source 1..25: mol_type = other DNA
acgctaatgccatctattaactagc

SEQ ID NO: 88 - DNA, 24 bp, synthetic construct
  misc_feature 1..24: note = Oligonucleotides used for Randomised DNA libraries constructionand sequencing
  source 1..24: mol_type = other DNA
gaaacaaccagaaacgctttttag

SEQ ID NO: 89 - DNA, 20 bp, synthetic construct
  misc_feature 1..20: note = Oligonucleotides used for Randomised DNA libraries constructionand sequencing
  source 1..20: mol_type = other DNA
ggcgaattcgagctcggtac

SEQ ID NO: 90 - DNA, 20 bp, synthetic construct
  misc_feature 1..20: note = Oligonucleotides used for Randomised DNA libraries constructionand sequencing
  source 1..20: mol_type = other DNA
ggtcgacggtatcgataagc

SEQ ID NO: 91 - DNA, 21 bp, synthetic construct
  misc_feature 1..21: note = Oligonucleotides used for Randomised DNA libraries constructionand sequencing
  source 1..21: mol_type = other DNA
gcaggcggaatgttgaaagag

SEQ ID NO: 92 - DNA, 22 bp, synthetic construct
  misc_feature 1..22: note = Oligonucleotides used for Randomised DNA libraries constructionand sequencing
  source 1..22: mol_type = other DNA
gttctggaggtttcgaatcttg

SEQ ID NO: 93 - DNA, 24 bp, synthetic construct
  misc_feature 1..24: note = Oligonucleotides used for Randomised DNA libraries constructionand sequencing
  source 1..24: mol_type = other DNA
gtgataatcgcctgcccgtttgac

SEQ ID NO: 94 - DNA, 22 bp, synthetic construct
  misc_feature 1..22: note = Oligonucleotides used for Randomised DNA libraries constructionand sequencing
  source 1..22: mol_type = other DNA
caagattcgaaacctccagaac

SEQ ID NO: 95 - DNA, 24 bp, synthetic construct
  misc_feature 1..24: note = Oligonucleotides used for Randomised DNA libraries constructionand sequencing
  source 1..24: mol_type = other DNA
gtcaaacgggcaggcgattatcac

SEQ ID NO: 96 - DNA, 51 bp, synthetic construct
  misc_feature 1..51: note = Oligonucleotides used for Randomised DNA libraries constructionand sequencing
  source 1..51: mol_type = other DNA
gcaggtcgactctagaggatcccacaagcttcgaaatccgccgaaccaatg

SEQ ID NO: 97 - DNA, 56 bp, synthetic construct
  misc_feature 1..56: note = Oligonucleotides used for Randomised DNA libraries constructionand sequencing
  source 1..56: mol_type = other DNA
gagctcggtacccggggatcccacgaattcactactggctactttgaaatacttcc

SEQ ID NO: 98 - DNA, 109 bp, synthetic construct
  misc_feature 1..109: note = Oligonucleotides used for Randomised DNA libraries constructionand sequencing
  misc_feature 25..32: note = n is a, c, g, or t
  misc_feature 42..46: note = n is a, c, g, or t
  misc_feature 57..61: note = n is a, c, g, or t
  misc_feature 72..79: note = n is a, c, g, or t
  source 1..109: mol_type = other DNA
cgaaatccgccgaaccaatgttgannnnnnnngcaggttcannnnntgtactctccnnnnnaaggtcaaatnnnnnnnntataggaagtatttcaaagtagccagtagt

SEQ ID NO: 99 - DNA, 109 bp, synthetic construct
  misc_feature 1..109: note = Oligonucleotides used for Randomised DNA libraries constructionand sequencing
  misc_feature 25..31: note = n is a, c, g, or t
  misc_feature 45..51: note = n is a, c, g, or t
  misc_feature 73..79: note = n is a, c, g, or t
  source 1..109: mol_type = other DNA
cgaaatccgccgaaccaatgttgannnnnnncgcaggttcaaatnnnnnnntctccttaataaggtcaaatgnnnnnnntataggaagtatttcaaagtagccagtagt

SEQ ID NO: 100 - DNA, 109 bp, synthetic construct
  misc_feature 1..109: note = Oligonucleotides used for Randomised DNA libraries constructionand sequencing
  misc_feature 25..31: note = n is a, c, g, or t
  misc_feature 52..58: note = n is a, c, g, or t
  misc_feature 73..79: note = n is a, c, g, or t
  source 1..109: mol_type = other DNA
cgaaatccgccgaaccaatgttgannnnnnncgcaggttcaaatcctgtacnnnnnnnaataaggtcaaatgnnnnnnntataggaagtatttcaaagtagccagtagt

SEQ ID NO: 101 - DNA, 184 bp, synthetic construct
  misc_feature 1..184: note = Oligonucleotides used for Randomised DNA libraries constructionand sequencing
  misc_feature 47..53: note = n is a, c, g, or t
  source 1..184: mol_type = other DNA
caagattcgaaacctccagaaccctccagaaacatggttgaaagaannnnnnntctccttaatcaaagcaataatccccgagaaatcaacattctcggggattatttttgtttttaactagaaaataactagaaagagctagttaatagatggcattagcgtgataatcgcctgcccgtttgac

SEQ ID NO: 102 - DNA, 184 bp, synthetic construct
  misc_feature 1..184: note = Oligonucleotides used for Randomised DNA libraries constructionand sequencing
  misc_feature 54..60: note = n is a, c, g, or t
  source 1..184: mol_type = other DNA
caagattcgaaacctccagaaccctccagaaacatggttgaaagaacctgtacnnnnnnnaatcaaagcaataatccccgagaaatcaacattctcggggattatttttgtttttaactagaaaataactagaaagagctagttaatagatggcattagcgtgataatcgcctgcccgtttgac

SEQ ID NO: 103 - DNA, 109 bp, synthetic construct
  misc_feature 1..109: note = Oligonucleotides used for Randomised DNA libraries constructionand sequencing
  misc_feature 32..41: note = n is a, c, g, or t
  source 1..109: mol_type = other DNA
cgaaatccgccgaaccaatgttgaattggtgnnnnnnnnnnaatcctgtactctccttaataaggtcaaatggtatccctataggaagtatttcaaagtagccagtagt

SEQ ID NO: 104 - DNA, 184 bp, synthetic construct
  misc_feature 1..184: note = Oligonucleotides used for Randomised DNA libraries constructionand sequencing
  misc_feature 34..43: note = n is a, c, g, or t
  source 1..184: mol_type = other DNA
caagattcgaaacctccagaaccctccagaaacnnnnnnnnnngaacctgtactctccttaatcaaagcaataatccccgagaaatcaacattctcggggattatttttgtttttaactagaaaataactagaaagagctagttaatagatggcattagcgtgataatcgcctgcccgtttgac

SEQ ID NO: 105 - DNA, 109 bp, synthetic construct
  misc_feature 1..109: note = Oligonucleotides used for Randomised DNA libraries constructionand sequencing
  misc_feature 35..44: note = n is a, c, g, or t
  source 1..109: mol_type = other DNA
cgaaatccgccgaaccaatgttgaattggtgcgcnnnnnnnnnncctgtactctccttaataaggtcaaatggtatccctataggaagtatttcaaagtagccagtagt

SEQ ID NO: 106 - DNA, 184 bp, synthetic construct
  misc_feature 1..184: note = Oligonucleotides used for Randomised DNA libraries constructionand sequencing
  misc_feature 40..46: note = n is a, c, g, or t
  source 1..184: mol_type = other DNA
caagattcgaaacctccagaaccctccagaaacatggttnnnnnnncctgtactctccttaatcaaagcaataatccccgagaaatcaacattctcggggattatttttgtttttaactagaaaataactagaaagagctagttaatagatggcattagcgtgataatcgcctgcccgtttgac

SEQ ID NO: 107 - DNA, 35 bp, synthetic construct
  misc_feature 1..35: note = Oligonucleotides used EMSA
  source 1..35: mol_type = other DNA
ggtattggaaagaacctgtactctccttgcgtaac

SEQ ID NO: 108 - DNA, 35 bp, synthetic construct
  misc_feature 1..35: note = Oligonucleotides used EMSA
  source 1..35: mol_type = other DNA
gttacgcaaggagagtacaggttctttccaatacc

SEQ ID NO: 109 - DNA, 28 bp, synthetic construct
  misc_feature 1..28: note = Oligonucleotides used EMSA
  source 1..28: mol_type = other DNA
gtttttaactagaaaataactagaattc

SEQ ID NO: 110 - DNA, 28 bp, synthetic construct
  misc_feature 1..28: note = Oligonucleotides used EMSA
  source 1..28: mol_type = other DNA
gaattctagttattttctagttaaaaac

SEQ ID NO: 111 - DNA, 40 bp, synthetic construct
  misc_feature 1..40: note = Oligonucleotides used EMSA
  source 1..40: mol_type = other DNA
cacgtcgtttttaactagaaaataactagaattccacgtc

SEQ ID NO: 112 - DNA, 40 bp, synthetic construct
  misc_feature 1..40: note = Oligonucleotides used EMSA
  source 1..40: mol_type = other DNA
gacgtggaattctagttattttctagttaaaaacgacgtg

SEQ ID NO: 113 - DNA, 40 bp, synthetic construct
  misc_feature 1..40: note = Oligonucleotides used EMSA
  source 1..40: mol_type = other DNA
tttttaactagaaaataactagaaagcgaccgagcggtcg

SEQ ID NO: 114 - DNA, 40 bp, synthetic construct
  misc_feature 1..40: note = Oligonucleotides used EMSA
  source 1..40: mol_type = other DNA
cgaccgctcggtcgctttctagttattttctagttaaaaa

SEQ ID NO: 115 - DNA, 40 bp, synthetic construct
  misc_feature 1..40: note = Oligonucleotides used EMSA
  source 1..40: mol_type = other DNA
tttttaactagaaaataactagaaagagctagttaataga

SEQ ID NO: 116 - DNA, 40 bp, synthetic construct
  misc_feature 1..40: note = Oligonucleotides used EMSA
  source 1..40: mol_type = other DNA
tctattaactagctctttctagttattttctagttaaaaa

SEQ ID NO: 117 - DNA, 40 bp, synthetic construct
  misc_feature 1..40: note = Oligonucleotides used EMSA
  source 1..40: mol_type = other DNA
tttttaactagaacgaccgagcggaggcggcacgctgtcg

SEQ ID NO: 118 - DNA, 40 bp, synthetic construct
  misc_feature 1..40: note = Oligonucleotides used EMSA
  source 1..40: mol_type = other DNA
cgacagcgtgccgcctccgctcggtcgttctagttaaaaa

SEQ ID NO: 119 - DNA, 40 bp, synthetic construct
  misc_feature 1..40: note = Oligonucleotides used EMSA
  source 1..40: mol_type = other DNA
gccgaccgagcggaataactagaaaggcggcacgctgtcg

SEQ ID NO: 120 - DNA, 40 bp, synthetic construct
  misc_feature 1..40: note = Oligonucleotides used EMSA
  source 1..40: mol_type = other DNA
cgacagcgtgccgcctttctagttattccgctcggtcggc

SEQ ID NO: 121 - DNA, 40 bp, synthetic construct
  misc_feature 1..40: note = Oligonucleotides used EMSA
  source 1..40: mol_type = other DNA
gccgaccgagcgggcggcacgctgagagctagttaataga

SEQ ID NO: 122 - DNA, 40 bp, synthetic construct
  misc_feature 1..40: note = Oligonucleotides used EMSA
  source 1..40: mol_type = other DNA
tctattaactagctctcagcgtgccgcccgctcggtcggc

SEQ ID NO: 123 - DNA, 40 bp, synthetic construct
  misc_feature 1..40: note = Oligonucleotides used EMSA
  source 1..40: mol_type = other DNA
gccgaccgagcggaataactagaaagagctagttaataga

SEQ ID NO: 124 - DNA, 40 bp, synthetic construct
  misc_feature 1..40: note = Oligonucleotides used EMSA
  source 1..40: mol_type = other DNA
tctattaactagctctttctagttattccgctcggtcggc

SEQ ID NO: 125 - DNA, 40 bp, synthetic construct
  misc_feature 1..40: note = Oligonucleotides used EMSA
  source 1..40: mol_type = other DNA
tttttaactagaacgaccgagcggagagctagttaataga

SEQ ID NO: 126 - DNA, 40 bp, synthetic construct
  misc_feature 1..40: note = Oligonucleotides used EMSA
  source 1..40: mol_type = other DNA
tctattaactagctctccgctcggtcgttctagttaaaaa

SEQ ID NO: 127 - DNA, 40 bp, synthetic construct
  misc_feature 1..40: note = Oligonucleotides used EMSA
  source 1..40: mol_type = other DNA
cacgtcgtgatcaactagatttttaactagaaaccacgtc

SEQ ID NO: 128 - DNA, 40 bp, synthetic construct
  misc_feature 1..40: note = Oligonucleotides used EMSA
  source 1..40: mol_type = other DNA
gacgtggtttctagttaaaaatctagttgatcacgacgtg

SEQ ID NO: 129 - DNA, 40 bp, synthetic construct
  misc_feature 1..40: note = Oligonucleotides used EMSA
  source 1..40: mol_type = other DNA
cacgtcgtgatcaactagattcgaccgagcggaccacgtc

SEQ ID NO: 130 - DNA, 40 bp, synthetic construct
  misc_feature 1..40: note = Oligonucleotides used EMSA
  source 1..40: mol_type = other DNA
gacgtggtccgctcggtcgaatctagttgatcacgacgtg

SEQ ID NO: 131 - DNA, 40 bp, synthetic construct
  misc_feature 1..40: note = Oligonucleotides used EMSA
  source 1..40: mol_type = other DNA
cacgtcgtgcgaccgagcggttttaactagaaaccacgtc

SEQ ID NO: 132 - DNA, 40 bp, synthetic construct
  misc_feature 1..40: note = Oligonucleotides used EMSA
  source 1..40: mol_type = other DNA
gacgtggtttctagttaaaaccgctcggtcgcacgacgtg

SEQ ID NO: 133 - DNA, 40 bp, synthetic construct
  misc_feature 1..40: note = Oligonucleotides used EMSA
  source 1..40: mol_type = other DNA
cacgtcgtgcgaccgagcggtgcggcacgctgaccacgtc

SEQ ID NO: 134 - DNA, 40 bp, synthetic construct
  misc_feature 1..40: note = Oligonucleotides used EMSA
  source 1..40: mol_type = other DNA
gacgtggtcagcgtgccgcaccgctcggtcgcacgacgtg

SEQ ID NO: 135 - DNA, 40 bp, synthetic construct; identical to SEQ ID NO: 111
  misc_feature 1..40: note = Oligonucleotides used EMSA
  source 1..40: mol_type = other DNA

SEQ ID NO: 136 - DNA, 35 bp, synthetic construct; identical to SEQ ID NO: 108
  misc_feature 1..35: note = Oligonucleotides used EMSA
  source 1..35: mol_type = other DNA

SEQ ID NO: 137 - DNA, 35 bp, synthetic construct
  misc_feature 1..35: note = Oligonucleotides used EMSA
  source 1..35: mol_type = other DNA
ggtattgttcaaatcctgtactctccttgcgtaac

SEQ ID NO: 138 - DNA, 35 bp, synthetic construct
  misc_feature 1..35: note = Oligonucleotides used EMSA
  source 1..35: mol_type = other DNA
gttacgcaaggagagtacaggatttgaacaatacc

SEQ ID NO: 139 - DNA, 35 bp, synthetic construct; identical to SEQ ID NO: 138
  misc_feature 1..35: note = Oligonucleotides used EMSA
  source 1..35: mol_type = other DNA

SEQ ID NO: 140 - DNA, 35 bp, synthetic construct
  misc_feature 1..35: note = Oligonucleotides used EMSA
  source 1..35: mol_type = other DNA
ggtattggaacccgcctgtactctccttgcgtaac

SEQ ID NO: 141 - DNA, 35 bp, synthetic construct
  misc_feature 1..35: note = Oligonucleotides used EMSA
  source 1..35: mol_type = other DNA
gttacgcaaggagagtacaggcgggttccaatacc

SEQ ID NO: 142 - DNA, 35 bp, synthetic construct
  misc_feature 1..35: note = Oligonucleotides used EMSA
  source 1..35: mol_type = other DNA
ggtattggaaagaacctgtaccgggcttgcgtaac

SEQ ID NO: 143 - DNA, 35 bp, synthetic construct
  misc_feature 1..35: note = Oligonucleotides used EMSA
  source 1..35: mol_type = other DNA
gttacgcaagcccggtacaggttctttccaatacc

SEQ ID NO: 144 - DNA, 35 bp, synthetic construct
  misc_feature 1..35: note = Oligonucleotides used EMSA
  source 1..35: mol_type = other DNA
ggtattgttcaaatcctgtaccgggcttgcgtaac

SEQ ID NO: 145 - DNA, 35 bp, synthetic construct
  misc_feature 1..35: note = Oligonucleotides used EMSA
  source 1..35: mol_type = other DNA
gttacgcaagcccggtacaggatttgaacaatacc

SEQ ID NO: 146 - DNA, 40 bp, synthetic construct
  misc_feature 1..40: note = Oligonucleotides used EMSA
  source 1..40: mol_type = other DNA
cacgtcgtgctcaactagagtcttaactagagaccacgtc

SEQ ID NO: 147 - DNA, 40 bp, synthetic construct
  misc_feature 1..40: note = Oligonucleotides used EMSA
  source 1..40: mol_type = other DNA
gacgtggtctctagttaagactctagttgagcacgacgtg

SEQ ID NO: 148 - DNA, 40 bp, synthetic construct
  misc_feature 1..40: note = Oligonucleotides used EMSA
  source 1..40: mol_type = other DNA
cacgtcgtgccgaactagggtccgaactagggaccacgtc

SEQ ID NO: 149 - DNA, 40 bp, synthetic construct
  misc_feature 1..40: note = Oligonucleotides used EMSA
  source 1..40: mol_type = other DNA
gacgtggtccctagttcggaccctagttcggcacgacgtg

SEQ ID NO: 150 - DNA, 40 bp, synthetic construct
  misc_feature 1..40: note = Oligonucleotides used EMSA
  source 1..40: mol_type = other DNA
gccgaccgagcggaataactagaaagattaactagctaga

SEQ ID NO: 151 - DNA, 40 bp, synthetic construct
  misc_feature 1..40: note = Oligonucleotides used EMSA
  source 1..40: mol_type = other DNA
tctagctagttaatctttctagttattccgctcggtcggc

SEQ ID NO: 152 - DNA, 40 bp, synthetic construct
  misc_feature 1..40: note = Oligonucleotides used EMSA
  source 1..40: mol_type = other DNA
tttttaactagaattctagttattagcgaccgagcggtcg

SEQ ID NO: 153 - DNA, 40 bp, synthetic construct
  misc_feature 1..40: note = Oligonucleotides used EMSA
  source 1..40: mol_type = other DNA
cgaccgctcggtcgctaataactagaattctagttaaaaa

SEQ ID NO: 154 - DNA, 19 bp, synthetic construct
  misc_feature 1..19: note = Oligonucleotides used for in vitro fluorescent recombination
  source 1..19: mol_type = other DNA
gaattcctgcagcccaagc

SEQ ID NO: 155 - DNA, 25 bp, synthetic construct
  misc_feature 1..25: note = Oligonucleotides used for in vitro fluorescent recombination
  source 1..25: mol_type = other DNA
gatgtagataatttttgggccaagg

SEQ ID NO: 156 - DNA, 21 bp, synthetic construct
  misc_feature 1..21: note = Oligonucleotides used to validate in vivo integration intotRNASER
  source 1..21: mol_type = other DNA
acagtgacgatctaacccttc

SEQ ID NO: 157 - DNA, 22 bp, synthetic construct
  misc_feature 1..22: note = Oligonucleotides used to validate in vivo integration intotRNASER
  source 1..22: mol_type = other DNA
tgactaatttgctttgttcctg

SEQ ID NO: 158 - DNA, 34 bp, synthetic construct
  misc_feature 1..34: note = Oligonucleotides used to validate in vivo integration intotRNASER
  source 1..34: mol_type = other DNA
catcatttttcttctttcaaattaatataaatgc

SEQ ID NO: 159 - DNA, 20 bp, synthetic construct
  misc_feature 1..20: note = Oligonucleotides used to validate in vivo integration intotRNASER
  source 1..20: mol_type = other DNA
caggaggaaaaggagtaagc

SEQ ID NO: 160 - DNA, 25 bp, synthetic construct
  misc_feature 1..25: note = Oligonucleotides used to validate in vivo integration intotRNASER
  source 1..25: mol_type = other DNA
acgctaatgccatctattaactagc

SEQ ID NO: 161 - protein, 83 aa, Lactobacillus delbrueckii
RVGDLCEMKWSDIVNNNNNNDVDGYLYNNNNNNNNVEQSKTHELRNSLSARLYEKQISDKNNFAQHLLGHKSDTMASBNQYRN

SEQ ID NO: 162 - protein, 84 aa, Lactobacillus delbrueckii
RIAEIARIRVKDISNNNNNNRTDGGRMLIHIGRNNNNNTKTLVHSARVGAARDMARAGVISPNNEIMQAGGWTNVNIVMNNYIR

SEQ ID NO: 163 - protein, 87 aa, Lactobacillus delbrueckii
RLSELVGLNNNDIKHLDNNNNNNNNNNNNNLESGEVWVMGKGSKHKLRHSFATHMLESSGDLRNNGVQELLGHANLSTTQNRITYTH

SEQ ID NO: 164 - protein, 86 aa, Lactobacillus delbrueckii
RVSELVGLTMSDISNNNNNNNNNNNNNNNNLRQGVVRVIGKGNKHVLRHAFATHLLNHGADLRNNVVQMLLGHSDLSTTQNITYTH

SEQ ID NO: 165 - protein, 87 aa, Lactobacillus delbrueckii
RWSEAEAETNNNNNLNNNNNNNNTQSQVMPYNNNNKITFTNTKSKKHVLRHTFASHFMMNGGNILNNVLKEILGHSTIEMTMNRYAH

SEQ ID NO: 166 - protein, 87 aa, Lactobacillus delbrueckii
RKRRSTSPGLVGHRLRSKEKSPSLKTLAYGLGGKYGIQPPKTKAHGFRHTFASLLITADPSIKPTDVQAILGHESIDITITMEIYMH

SEQ ID NO: 167 - protein, 87 aa, Lactobacillus delbrueckii
RKGEALALDWSDIDYNDQGKISVTKTLAYGLGGKYGIQPPKTKAHGFRHTFASLLITADPSIKPTDVQAILGHESIDITITMEIYMH

SEQ ID NO: 168 - DNA, 30 bp, Lactobacillus delbrueckii
  source 1..30: mol_type = genomic DNA
atggttgaaagaacctgtactctccttaat

SEQ ID NO: 169 - DNA, 30 bp, Lactobacillus delbrueckii
  source 1..30: mol_type = genomic DNA
cgcaggttcaaatcctgtactctccttaat

SEQ ID NO: 170 - DNA, 30 bp, Lactobacillus delbrueckii
  source 1..30: mol_type = genomic DNA
ccacggttcaaatcctgtactctccttaat

SEQ ID NO: 171 - DNA, 30 bp, Lactobacillus delbrueckii
  source 1..30: mol_type = genomic DNA
gaggatcccacatcctgtactctccttaat

SEQ ID NO: 172 - DNA, 20 bp, Lactobacillus delbrueckii
  source 1..20: mol_type = genomic DNA
aatcctgtactctccttaat

SEQ ID NO: 173 - DNA, 20 bp, Lactobacillus delbrueckii
  misc_feature 1..5: note = n is a, c, g, or t
  misc_feature 16..20: note = n is a, c, g, or t
  source 1..20: mol_type = genomic DNA
nnnnntgtactctccnnnnn

SEQ ID NO: 174 - DNA, 21 bp, Lactobacillus delbrueckii
  misc_feature 4..13: note = n is a, c, g, or t
  source 1..21: mol_type = genomic DNA
cgcnnnnnnnnnncctgtact

SEQ ID NO: 175 - DNA, 21 bp, Lactobacillus delbrueckii
  misc_feature 1..10: note = n is a, c, g, or t
  source 1..21: mol_type = genomic DNA
nnnnnnnnnnaatcctgtact

SEQ ID NO: 176 - DNA, 21 bp, Lactobacillus delbrueckii
  source 1..21: mol_type = genomic DNA
cgcaggttcaaatcctgtact

SEQ ID NO: 177 - DNA, 19 bp, Lactobacillus delbrueckii
  misc_feature 10..16: note = n is a, c, g, or t
  source 1..19: mol_type = genomic DNA
aacctgtacnnnnnnnaat

SEQ ID NO: 178 - DNA, 19 bp, Lactobacillus delbrueckii
  misc_feature 10..16: note = n is a, c, g, or t
  source 1..19: mol_type = genomic DNA
atcctgtacnnnnnnnaat

SEQ ID NO: 179 - DNA, 21 bp, Lactobacillus delbrueckii
  misc_feature 15: note = n is a, c, g, or t
  source 1..21: mol_type = genomic DNA
gaaagaacctgtacnctcctt

SEQ ID NO: 180 - DNA, 21 bp, Lactobacillus delbrueckii
  source 1..21: mol_type = genomic DNA
ttcaaatcctgtactctcctt

SEQ ID NO: 181 - DNA, 21 bp, Lactobacillus delbrueckii
  source 1..21: mol_type = genomic DNA
ttcaaatcctgtactctcctt

SEQ ID NO: 182 - DNA, 21 bp, Lactobacillus delbrueckii
  source 1..21: mol_type = genomic DNA
gaaagaacctgtactctcctt

SEQ ID NO: 183 - DNA, 21 bp, Lactobacillus delbrueckii
  misc_feature 15: note = n is a, c, g, or t
  source 1..21: mol_type = genomic DNA
ttcaaatcctgtacnctcctt

SEQ ID NO: 184 - DNA, 19 bp, Lactobacillus delbrueckii
  source 1..19: mol_type = genomic DNA
atcctgtactctccttaat

SEQ ID NO: 185 - DNA, 19 bp, Lactobacillus delbrueckii
  source 1..19: mol_type = genomic DNA
aacctgtactctccttaat

SEQ ID NO: 186-187 - skipped (2 entries)

SEQ ID NO: 188 - DNA, 21 bp, Lactobacillus delbrueckii
  source 1..21: mol_type = genomic DNA
gaaagaatgccaactctcctt

SEQ ID NO: 189 - DNA, 20 bp, Lactobacillus delbrueckii
  source 1..20: mol_type = genomic DNA
ttcaattgccaactctcctt

SEQ ID NO: 190 - DNA, 21 bp, Lactobacillus delbrueckii
  source 1..21: mol_type = genomic DNA
gaaagaatttcggctctcctt

SEQ ID NO: 191 - DNA, 21 bp, Lactobacillus delbrueckii
  source 1..21: mol_type = genomic DNA
ttcaaattttcggctctcctt

SEQ ID NO: 192 - DNA, 21 bp, Lactobacillus delbrueckii
  source 1..21: mol_type = genomic DNA
gaaagaaaaggttttctcctt

SEQ ID NO: 193 - DNA, 21 bp, Lactobacillus delbrueckii
  source 1..21: mol_type = genomic DNA
ttcaaataaggttttctcctt

SEQ ID NO: 194 - DNA, 16 bp, Lactobacillus delbrueckii
  source 1..16: mol_type = genomic DNA
aacctgtactctcctt

SEQ ID NO: 195 - DNA, 16 bp, Lactobacillus delbrueckii
  source 1..16: mol_type = genomic DNA
atcctgtactctcctt

SEQ ID NO: 196 - skipped

SEQ ID NO: 197 - DNA, 11 bp, Lactobacillus delbrueckii
  source 1..11: mol_type = genomic DNA
aatgccaactc

SEQ ID NO: 198 - DNA, 11 bp, Lactobacillus delbrueckii
  source 1..11: mol_type = genomic DNA
aaaaggttttc

SEQ ID NO: 199 - DNA, 11 bp, Lactobacillus delbrueckii
  source 1..11: mol_type = genomic DNA
aatttcggctc

SEQ ID NO: 200 - skipped

SEQ ID NO: 201 - DNA, 27 bp, Lactobacillus delbrueckii
  source 1..27: mol_type = genomic DNA
aggttcaaatcctgtactctccttaat

SEQ ID NO: 202 - DNA, 20 bp, Lactobacillus delbrueckii
  misc_feature 7..13: note = n is a, c, g, or t
  source 1..20: mol_type = genomic DNA
ddbrrwnnnnnnndyyyvhb

SEQ ID NO: 203 - DNA, 27 bp, Lactobacillus delbrueckii
  source 1..27: mol_type = genomic DNA
gttgaaagaacctgtactctccttaat

SEQ ID NO: 204 - DNA, 21 bp, Lactobacillus delbrueckii
  misc_feature 8..14: note = n is a, c, g, or t
  source 1..21: mol_type = genomic DNA
ddbdrdwnnnnnnndyyyvhy

SEQ ID NO: 205 - DNA, 13 bp, Lactobacillus delbrueckii
  source 1..13: mol_type = genomic DNA
ttcaatcctgtac

SEQ ID NO: 206 - skipped

SEQ ID NO: 207 - DNA, 14 bp, Lactobacillus delbrueckii
  source 1..14: mol_type = genomic DNA
cctgtactctcctt

SEQ ID NO: 208 - skipped

SEQ ID NO: 209 - DNA, 14 bp, Lactobacillus delbrueckii
  source 1..14: mol_type = genomic DNA
gaaagaacctgtac

SEQ ID NO: 210 - skipped

SEQ ID NO: 211 - DNA, 14 bp, Lactobacillus delbrueckii
  source 1..14: mol_type = genomic DNA
cctgtactctcctt

SEQ ID NO: 212 - skipped

SEQ ID NO: 213 - DNA, 24 bp, Lactobacillus delbrueckii
  source 1..24: mol_type = genomic DNA
ttcaaatcctgtactctccttaat

SEQ ID NO: 214 - DNA, 24 bp, Lactobacillus delbrueckii
  source 1..24: mol_type = genomic DNA
gaaagaacctgtactctccttaat

SEQ ID NO: 215 - DNA, 24 bp, Lactobacillus sakei
  source 1..24: mol_type = genomic DNA
ttcaaatcctgtactctcctttta

SEQ ID NO: 216 - DNA, 24 bp, Lactobacillus sakei
  source 1..24: mol_type = genomic DNA
gaaagaacctgtactctcctttta

SEQ ID NO: 217 - DNA, 23 bp, Lactobacillus acidophilus
  source 1..23: mol_type = genomic DNA
ttcaatcctgtacactccttttt

SEQ ID NO: 218 - DNA, 21 bp, Lactobacillus acidophilus
  source 1..21: mol_type = genomic DNA
gaaagaacctgtacactcctt

SEQ ID NO: 219 - DNA, 24 bp, Lactococcus lactis
  source 1..24: mol_type = genomic DNA
ttcaaatccccttgactcctttta

SEQ ID NO: 220 - DNA, 24 bp, Lactococcus lactis
  source 1..24: mol_type = genomic DNA
gaaagaaccccttgactcctttta

SEQ ID NO: 221 - DNA, 23 bp, Streptococcus mutans
  source 1..23: mol_type = genomic DNA
ttcaaatcccgtcctttcttaac

SEQ ID NO: 222 - DNA, 24 bp, Streptococcus mutans
  source 1..24: mol_type = genomic DNA
gaaagaacccgtcctttccttaac

SEQ ID NO: 223 - DNA, 24 bp, Lactobacillus reuteri
  source 1..24: mol_type = genomic DNA
ttcaaatccccttgtctccttaga

SEQ ID NO: 224 - DNA, 24 bp, Lactobacillus reuteri
  source 1..24: mol_type = genomic DNA
gaaagaaccccttgtctccttaga

SEQ ID NO: 225 - DNA, 24 bp, synthetic construct
  misc_feature 1..24: note = figure 10
  source 1..24: mol_type = other DNA
ttcaaatccccttgtctccataga

SEQ ID NO: 226 - DNA, 24 bp, synthetic construct
  misc_feature 1..24: note = figure 10
  source 1..24: mol_type = other DNA
gaaagaaccccttgtctccataga

SEQ ID NO: 227 - DNA, 24 bp, Leuconostoc mesenteroides
  source 1..24: mol_type = genomic DNA
ttcaaatcccgctagctcctttat

SEQ ID NO: 228 - DNA, 24 bp, Leuconostoc mesenteroides
  source 1..24: mol_type = genomic DNA
gaaagaacccgctagctcctttat

SEQ ID NO: 229 - DNA, 23 bp, synthetic construct
  misc_feature 1..23: note = figure 10
  source 1..23: mol_type = other DNA
ttcaatcctctctccgccacttt

SEQ ID NO: 230 - DNA, 24 bp, synthetic construct
  misc_feature 1..24: note = figure 10
  source 1..24: mol_type = other DNA
gaaagaacctcgctccgccacttt

SEQ ID NO: 231 - DNA, 23 bp, Enterococcus faecalis
  source 1..23: mol_type = genomic DNA
ttcaaatccctatcctccgtacc

SEQ ID NO: 232 - DNA, 24 bp, Enterococcus faecalis
  source 1..24: mol_type = genomic DNA
gaaagaacccttatcctccgtacc

SEQ ID NO: 233 - DNA, 21 bp, Lactobacillus delbrueckii
  source 1..21: mol_type = genomic DNA; sub_species = subsp. bulgaricus
ttcaaatcctgtactctcctt

SEQ ID NO: 234 - DNA, 21 bp, Escherichia coli
  source 1..21: mol_type = genomic DNA
ttcaaatccccctctctccgc

SEQ ID NO: 235 - DNA, 21 bp, Lactococcus lactis
  source 1..21: mol_type = genomic DNA
ttcaaatccccttgactcctt

SEQ ID NO: 236 - DNA, 21 bp, Escherichia coli
  source 1..21: mol_type = genomic DNA
gaaagaatttcggctctcctt

SEQ ID NO: 237 - DNA, 21 bp, Escherichia coli
  source 1..21: mol_type = genomic DNA
gttaaattgccaacgcttatt

SEQ ID NO: 238 - DNA, 21 bp, Escherichia coli
  source 1..21: mol_type = genomic DNA
gaaagaatgccaactctcctt

SEQ ID NO: 239 - DNA, 21 bp, Escherichia coli
  source 1..21: mol_type = genomic DNA
gataaataaggttttcccctg

SEQ ID NO: 240 - DNA, 21 bp, Escherichia coli
  source 1..21: mol_type = genomic DNA
gaaagaaaaggttttctcctt

SEQ ID NO: 241 - DNA, 21 bp, Escherichia coli
  source 1..21: mol_type = genomic DNA
ttcgggatttcggctctcctt

SEQ ID NO: 242 - DNA, 21 bp, Escherichia coli
  source 1..21: mol_type = genomic DNA
gaaagaatttcggcgctccac

SEQ ID NO: 243 - DNA, 21 bp, Escherichia coli
  source 1..21: mol_type = genomic DNA
gttaaattgccaactctcctt

SEQ ID NO: 244 - DNA, 21 bp, Escherichia coli
  source 1..21: mol_type = genomic DNA
gaaagaatgccaacgcttatt

SEQ ID NO: 245 - DNA, 21 bp, Escherichia coli
  source 1..21: mol_type = genomic DNA
gataaataaggttttctcctt

SEQ ID NO: 246 - DNA, 21 bp, Escherichia coli
  source 1..21: mol_type = genomic DNA
gaaagaaaaggttttcccctg

SEQ ID NO: 247 - DNA, 30 bp, Lactobacillus delbrueckii
  source 1..30: mol_type = genomic DNA
cgcaggttcaaatcctgtacgctccttaat

SEQ ID NO: 248 - DNA, 21 bp, synthetic construct
  source 1..21: mol_type = other DNA
ttcacatcctgtactctcctt

SEQ ID NO: 249 - DNA, 21 bp, synthetic construct
  source 1..21: mol_type = other DNA
ttcatatcctgtactctcctt

SEQ ID NO: 250 - DNA, 21 bp, synthetic construct
  source 1..21: mol_type = other DNA
ttcaaaccctgtactctcctt

SEQ ID NO: 251 - DNA, 21 bp, synthetic construct
  source 1..21: mol_type = other DNA
ttcacagcctgtactctcctt